(19) United States
(12) Patent Application Publication
(10) Pub. No.: US 2026/0295030 A1
(43) Pub. Date: Oct. 1, 2026

(54) IMPROVED EXPRESSION OF SURFACE-DISPLAYED ANTIGENS

(71) Applicant: INPROTHER APS, Copenhagen (DK)

(72) Inventors: Anne-Marie Carola ANDERSSON, Copenhagen (DK); Peter Johannes HOLST, Søborg (DK); Emeline RAGONNAUD, Søborg (DK); Lasse NEUKIRCH, Copenhagen (DK)

(73) Assignee: INPROTHER APS, Copenhagen (DK)

(21) Appl. No.: 19/477,262

(22) PCT Filed: Apr. 22, 2024
(86) PCT No.: PCT/EP2024/060972

§ 371 Date: Oct. 21, 2025

(30) Foreign Application Priority Data
Apr. 21, 2023 (EP) 23169279.9

(51) Int. Cl. A61K 39/21; A61K 9/00; A61K 39/00; A61P 35/00; C07K 14/15; C12N 15/11

(52) U.S. Cl. CPC A61K 39/21; A61K 9/0019; A61P 35/00; C07K 14/15; C12N 15/11; A61K 2039/5258; A61K 2039/53; A61K 2039/54; A61K 2039/545; A61K 2039/55555; A61K 2039/585; A61K 2039/6018; C12N 2740/10023; C12N 2740/10034

(57) ABSTRACT

The present invention relates to a composition comprising a transfection agent and an mRNA which encodes at least a human endogenous retrovirus (HERV) envelope protein or an immunogenic part thereof with a mutation in the immune-suppressive domain (ISD) that reduces its immunosuppressive property. The present invention also relates to the use of this composition in the manufacture of medicaments and in therapeutic and prophylactic treatment.

### CROSS REFERENCE TO RELATED APPLICATIONS

This application is a 35 U.S.C. § 371 National Stage of International Patent Application No. PCT/EP2024/060972, filed Apr. 22, 2024, claiming benefit from European Patent Application No. 23169279.9, filed Apr. 21, 2023, the disclosures of which are incorporated herein in their entirety by reference, and priority is claimed to each of the foregoing.

### REFERENCE TO AN ELECTRONIC SEQUENCE LISTING

The contents of the electronic sequence listing (1195-4 PCT sequence protocol 2024-04-22.xml; Size: 83,635 bytes; and Date of Creation: Apr. 22, 2024) is herein incorporated by reference in its entirety.

### BACKGROUND OF THE INVENTION

Although immune cells are able to detect and kill tumor cells, this system is not always functional, as evident from the almost 9 million annual deaths worldwide due to cancer. Vaccination approaches to induce specific immune responses against tumor cells is a relatively old topic in cancer immunotherapies but is still under development and just recently started to yield relevant results. One vaccination strategy involves the vaccination with attenuated tumor cells, e.g., irradiated autologous tumors or allogeneic tumor cell lines, often secreting the granulocyte-macrophage colony-stimulating factor (GM-CSF). In both cases the injected material encompasses cancer-antigens that are likely present in the actual tumor. Other vaccination strategies include the administration of peptides or proteins to induce specific immune responses. These antigens are either injected directly in combination with an adjuvant, or are encoded by DNA plasmids or viral vectors.

Although immunotherapy approaches are constantly improving, broadly acting and highly efficient vaccines are still missing. A particular reason for this is the previously described immunosuppression by tumor cells.

Endogenous retroviruses (ERVs) are the evidence of ancient infections with retroviruses in distant ancestors. Upon infection, viral RNA was reverse transcribed into parvoviral DNA, which was integrated into the host genome. Eventually, the provirus was integrated into cells of the germ line and became inheritable, giving rise to endogenous retroviruses. Over millions of years the viral DNA was passed down generations and became fixed in the populations. Today, every human genome consists of about 8% endogenous retroviral DNA, but these are just relics of the former retrovirus. Due to mutations, deletions and insertions most of the retroviral genes became inactivated or got completely lost from the genome. Today, no functional, full-length endogenous retrovirus is present in humans anymore. However, ERVs underwent duplication processes leading to the integration of several copies into the host genome with distinct functional proteins. Thus, in some cases the multitude of homologous ERVs has still the potential to produce viral particles. The human ERV type K (HERV-K, HML2) is one of the most recently acquired ERVs in the human genome and members of this family remained full-length open reading-frames for almost all viral proteins.

Different studies have highlighted a connection between ERV expression and the development and progression of cancer. The detection of ERVs in human tumors opened a new field in anti-cancer therapies with the prospect of new vaccination strategies. A prominent example for a human ERV (HERV) is HERV type K (HERV-K) that is associated with prostate cancer, breast cancer, ovarian cancer, lymphomas, melanomas, leukemia and sarcomas. Further examples are HERV-H expressed in colorectal cancer and Syncytin-1 in testicular cancer, ovarian cancer, breast cancer, lymphomas and leukemia.

It is not always easy to determine whether the expression of ERV proteins is a cause or a consequence of the developing tumor. Nevertheless, it is known that conditions within the cancer cell enable expression of ERVs. The general state of hypomethylation in tumor cells promotes activation of ERV genes that are usually silenced in healthy cells by DNA methylation (Downey, R. F., et al., Human endogenous retrovirus K and cancer: Innocent bystander or tumorigenic accomplice?Int J Cancer, 2015. 137(6): p. 1249-57. and Gimenez, J., et al., Custom human endogenous retroviruses dedicated microarray identifies self-induced HERV-W family elements reactivated in testicular cancer upon methylation control. Nucleic Acids Res, 2010. 38(7): p. 2229-46). Also, exogenous factors can promote ERV expression. Activation of human ERVs was for example observed due to viral infections. HERV-W expression was detected after influenza and herpes simplex virus infection (Nellaker, C., et al., Transactivation of elements in the human endogenous retrovirus W family by viral infection. Retrovirology, 2006. 3: p. 44) while HERV-K was present after Epstein-Barr virus infection (Sutkowski, N., et al., Epstein-Barr virus transactivates the human endogenous retrovirus HERV-K18 that encodes a superantigen. Immunity, 2001. 15(4): p. 579-89). Regardless of the mechanism that leads to ERV expression, cancer cells maintain activation of these proteins by a selection pressure, indicating a beneficial effect of ERVs in tumors (Leong, S. P., et al., Expression and modulation of a retrovirus-associated antigen by murine melanoma cells. Cancer Res, 1988. 48(17): p. 4954-8.)

Not only human tumors are associated with ERV proteins, but also murine cancer cells express ERVs. This provides a perfect model organism to study effects of ERVs on tumor progression and to test ERV-targeting therapy approaches. One ERV model is the melanoma associated retrovirus (MelARV), which originates from a provirus of the murine leukemia virus (MuLV) present in the mouse genome. Most inbred mouse strains contain one or two inactive MuLV copies (Li, M., et al., Sequence and insertion sites of murine melanoma-associated retrovirus. J Virol, 1999. 73(11): p. 9178-86.) However, the AKR mouse strain has three insertions in the genome and is characterized by a high production of MuLV early in life causing frequent incidences of spontaneous lymphomas. Other mouse strains, like the C57BL/6, spontaneously produce MuLV particles only later in life. Several other murine cancer models likewise express MuLV/MelARV, similar to human ERVs.

As the immune system of a viral host is a natural defense mechanism against infections, many viruses and especially retroviruses have developed strategies to escape this surveillance. One mechanism that can be seen throughout different virus families [Duch et al., WO2013/050048] is the development of an immunosuppressive domain in the envelope proteins (Env) causing a suppression of the immune system on different levels. Immune cells including natural killer (NK), CD8 T or regulatory T (Treg) cells can be affected by viruses containing an ISD [Schlecht-Louf et al. (2010)].

Many ERVs contain proteins with immunosuppressive domains (ISD) and such a domain can also be found in the MelARV Env protein (Schlecht-Louf, G., et al., Retroviral infection in vivo requires an immune escape virulence factor encrypted in the envelope protein of oncoretroviruses. Proc Natl Acad Sci USA, 2010. 107(8): p. 3782-7 and Mangeney, M. and T. Heidmann, Tumor cells expressing a retroviral envelope escape immune rejection in vivo. Proc Natl Acad Sci USA, 1998. 95(25): p. 14920-5.). The importance of the ISD in MuLV or MelARV has been shown by introducing murine leukemia virus Env proteins into tumor cells that are normally rejected by immune cells (Mangeney, M. and T. Heidmann, Tumor cells expressing a retroviral envelope escape immune rejection in vivo. Proc Natl Acad Sci USA, 1998. 95(25): p. 14920-5). Env transduced tumor cells grew more rapidly despite the additional exogenous antigen. This observation was explained by a local immunosuppressive effect mediated by the Env protein. The ISD is affecting both the innate and adaptive immune system, as shown by inhibition of macrophages, NK cells and T cells alike (Lang, M. S., et al., Immunotherapy with monoclonal antibodies directed against the immunosuppressive domain of p15E inhibits tumor growth. Clin Exp Immunol, 1995. 102(3): p. 468-75). Furthermore, an effect on the regulatory T cell subset has been suggested that in turn suppresses other immune cells (Mangeney, M., et al., Endogenous retrovirus expression is required for murine melanoma tumor growth in vivo. Cancer Res, 2005. 65(7): p. 2588-91). The detailed mechanism of immunosuppression by the ISD is not completely understood yet, but the effect seems mostly mediated by the CKS-17 peptide within the ISD. CKS-17 has diverse effects on the immune system, mostly by altering cytokine expression (Haraguchi, S., R. A. Good, and N. K. Day-Good, A potent immunosuppressive retroviral peptide: cytokine patterns and signaling pathways. Immunol Res, 2008. 41(1): p. 46-5⁵.).

One of the first therapeutic approaches to target ERV-expressing tumor cells included the administration of monoclonal antibodies. Thus, antibodies targeting HERV-K Env were able to reduce tumor growth of breast cancer cell lines. Wang-Johanning et al. showed that the observed effect of anti-HERV-K Env monoclonal antibodies was mediated by alteration of the cancer cell cycle and increased apoptosis. Another possible effect of such antibodies, not tested by Wang-Johanning et al. (Wang-Johanning, F., et al., Immunotherapeutic potential of anti-human endogenous retrovirus-K envelope protein antibodies in targeting breast tumors. J Natl Cancer Inst, 2012. 104(3): p. 189-210), could be the prevention of immunosuppression. Like MelARV Env, the HERV-K Env protein contains an ISD and has immune modulating functions (Morozov, V. A., V. L. Dao Thi, and J. Denner, The transmembrane protein of the human endogenous retrovirus-K (HERV-K) modulates cytokine release and gene expression. PLoS One, 2013. 8(8): p. e70399). The approach tested by Wang-Johanning et al. included xenograft tumors in immunodeficient athymic mice. Thus, the effect of HERV-K could only affect innate immune cells, such as NK cells.

Another part of the adaptive immune response that can help to eradicate tumors by targeting ERVs includes T cells. For instance, adoptively transferred T cells against a MuLV Env epitope in combination with IL-2 were able to eradicate lung metastases of melanoma cells (Yang, J. C. and D. Perry-Lalley, The envelope protein of an endogenous murine retrovirus is a tumor-associated T-cell antigen for multiple murine tumors. J Immunother, 2000. 23(2): p. 177-83). Similar experiments were performed in humanized mouse models for HERV-K. T cells were genetically modified to express on their surface a chimeric antigen receptor (CAR) that recognizes HERV-K Env on cancer cells. The cytotoxic CAR+ T-cells were able to lyse tumor cells and prevented metastases as well as tumor growth.

In addition to the direct injection of antibodies or T cells, a more practical, cheaper and efficient strategy is the induction of immune responses by vaccination. A simple approach is the vaccination with virus-encoded antigens. However, this method is rather cumbersome as DCs have to be isolated and cultured first before they are pulsed with a defined HLA-restricted peptide and are re-injected into mice or patients.

One elegant vaccination strategy is the presentation of antigens (e.g. viral envelope proteins) to the immune system on virus-like particles (VLPs), which are encoded in a nucleotide comprised by the vaccine. These particles do not contain viral nucleic acids and are therefore non-infectious. Nevertheless, VLPs are highly immunogenic and displayed proteins are presented in a natural context. For example, the viral Env protein integrated in VLPs is presented on a virus-like surface, which promotes correct folding and conformation. In addition to the advantage of a strong immunogenicity, the vaccination strategy with VLPs includes also practical benefits. Thus, VLPs are relatively easy to produce as they are built from just a single or few proteins and production can be performed in cell cultures. Bayer et al. (2010) showed that only the combination of encoded and capsid presented antigens was able to increase the level of functional antibodies. This observation was assigned to the fact that while the presentation on the adenoviral capsid helped to cross-link B cell receptors, encoded antigens were required for an essential CD4+ T cell responses promoting affinity maturation of B cells. With this vaccination strategy Bayer et al. were able to reduce viral load of F-MLV after challenge. However, no indication of increased CD8+ T cell responses against the target antigen could be observed. Shoji et al. primarily focused on the optimization of an adenovirus-based HIV vaccine and investigated the in-situ formation of Gag based VLPs. In their study such a setting showed the highest immune responses compared to other display strategies that did not promote in situ formation of VLPs [Shoji et al., 2012].

In order to vaccinate against viruses or virus-related disease (e.g. ERV expressing cancer), the whole Env protein should ideally be displayed to the immune system to ensure an immune response against a full protein target. However, as the Env protein contains the ISD, the vaccine itself has an immunosuppressive ability, undesired for an immunization approach. To circumvent this drawback, mutations were introduced into the ISD to maintain natural conformation of the target protein while at the same time preventing the immunosuppression. Similarly, US2012189647 relates to a mutated envelope protein resulting from mutation of an immunosuppressive domain of a transmembrane subunit of a wild type envelope protein.

One of the firsts to test inactivating mutations in the ISD of viral proteins was Schlecht-Louf et al. [Schlecht-Louf et al. (2010)]. Based on comparison studies between the immunosuppressive syncytin-2 and the non-immunosuppressive syncytin-1 [Mangeney et al. (2007)], Schlecht-Louf et al. identified mutations that disable the activity of the ISD without ablating the general structure and functionality of the Env protein. This mutation strategy was applied to proteins of other viral origins (e.g. HTLV and XMRV) and more extensively tested for the Friend murine leukemia virus (F-MLV). The study did not only reveal the suppression of both NK and T cells by the ISD but showed also that a live-attenuated F-MLV virus comprising the mutated ISD in the Env protein served as a vaccine against the same virus with a WT ISD sequence. The protection was due to increased antibody levels as well as T cell responses against F-MLV epitopes. Their discovery was finally manifested in the patent application WO 2011/092199 with focus on the Xenotropic murine leukemia virus-related virus (XMRV) that has been related to human prostate cancer and chronic fatigue syndrome. Hence, WO 2011/092199 relates to ISD mutations specifically in the XMRV and to the utilization of such ISD mutated viruses for vaccination strategies.

Another application of ISD mutation was described in the patent application WO 2014/195510. In this case a mutation of the ISD was introduced in the Feline Immunodeficiency Virus (FIV) in order to decrease immunosuppression by the virus while still maintaining its natural conformation. WO 2014/195510 describes that specific mutations increased antibody responses against the FIV Env protein when administered in a vaccination approach, bound to MBP or transduced in engrafted tumor cells. Thus, WO 2014/195510 relates to mutations in the ISD of FIV Env and the use of such mutated proteins in vaccination approaches against infection with FIV or other lentiviruses.

Another approach, addressing a broader spectrum of ISD mutations in viral Env protein, is described in the patent application WO 2013/050048. In particular WO 2013/050048 relates to the generation of antigens by first identifying ISDs in enveloped RNA viruses and subsequently mutating these domains to decrease immunosuppression during vaccination. The ISD identification strategy is based on 4 parameters which are: 1) the peptide is located in the fusion protein of enveloped RNA viruses, 2) the peptide is capable of interacting with membranes, 3) a high degree of homology in the primary structure (sequence) of the peptide exists either within the Order, Family, Subfamily, Genus, or Species of viruses, 4) the position at the surface of the fusion protein at a given conformation is a feature of immunosuppressive domains, revealed by the 3D structure or antibody staining. After identification of a potential ISD in a viral Env of interest, the immunosuppressive function was validated and subsequently, mutations were introduced in the ISDs and reduction of immunosuppression of at least 25% was confirmed. Overall, WO 2013/050048 describes the identification of ISDs in enveloped RNA viruses, the generation of ISD mutated peptides, as well as the utilization of said peptides as vaccines and the generation of antibodies.

Despite previous strategies of mutating ISDs in viral Env proteins using adenovirus to encode and display viral antigens, past vaccination strategies employing ISD mutations mainly aimed at preventing viral infections [Schlecht-Louf et al. 2010; WO 2011/092199; WO 2014/195510; US20110305749; WO 2014/195510]. Therefore, there is still a need to break tolerance to self-antigens. Such breaking of tolerance may be facilitated by densely surface protein coated particles (such as VLPs). Dense coating with membrane associated viral glycoproteins is increased with increased cell surface expression prior to incorporation of proteins in virus-like particles budding from the cell surface. An additional benefit of robust cell surface expression includes the ability to directly stimulate B cell recognition (see also Ferapontov, A., Omer, M., Baudrexel, I. et al. Antigen footprint governs activation of the B cell receptor. Nat Commun 14, 976 (2023)).

Moreover, the system of in situ synthesis of virus-like particles has been used before [Luo et al. (2003); Sohji et al. (2011); Andersson et al. (2016); Andersson & Holst (2016); Andersson et al. (2017)], but there is a need for alternative methods for improved antigen display besides the use of VLPs for situations in which VLPs are not applicable. Moreover, there is also a need for an efficient system allowing the efficient production of an antigen or antigen-VLP, for instance HERV-K VLPs. The establishment of such a system ideally requires the previous effective introduction of the system into a host cell or organism. Thus, there is a need for providing improved delivery methods and delivery means for administering polynucleotides to elicit an immune response in a subject and/or to treat a disease that involves the expression of a protein e.g., a cancer marker protein, in a cell of that subject.

### SUMMARY OF THE INVENTION

As outlined above, there is, with regard to ERVs, a need for efficient surface display of endogenous retroviral antigens for example for vaccination of a subject against endogenous tumor potential and/or tumor progression. The present invention aims at producing such an effective vaccine for the prophylaxis and/or treatment of a disease caused by an endogenous retrovirus. The composition of the invention comprising the mRNA of the invention provides an improved surface display of the encoded antigens on host cells, incorporation into virus-like-particles and increasing stimulation of the immune system.

Thus, in one aspect the present invention relates to a composition comprising a transfection agent and an mRNA which encodes at least a human endogenous retrovirus (HERV) envelope protein or an immunogenic part thereof; wherein said HERV envelope protein comprises an immune-suppressive domain (ISD); wherein the HERV Env protein comprises a mutated immune-suppressive domain (ISD) that reduces its immunosuppressive property compared to the wildtype ISD and/or allows it to be more efficiently presented on the cell surface. In one embodiment, the composition comprises lipid nanoparticles (LNPs) that comprise said mRNA.

In another aspect the present invention relates to the composition of the invention for use as a medicament.

In another aspect the present invention relates to the use of the composition for the manufacture of a medicament.

In yet another aspect the present invention relates to the composition for use in the prophylaxis or treatment of a disease, preferably for immunizing a subject against a disease.

In yet another aspect the present invention relates to the use of the composition for the manufacture of a medicament for prophylaxis and/or therapeutic treatment of a disease, preferably for immunizing a subject against a disease. In yet another aspect the present invention relates to a method of treatment and/or prophylaxis of a disease, preferably of immunizing a subject against a disease, comprising administering to the subject (preferably a therapeutically effective amount) of the composition of the invention. In one embodiment, the disease is preferably selected from the group consisting of cancer, HIV and/or associated disorders, rheumatic diseases, neurodegenerative diseases, aging associated diseases, diseases associated with HERV reactivation, diseases associated with HERV reactivation, chronic inflammation multiple sclerosis, ALS associated with TDP-43, Alzheimer's disease associated with Tau expression, ALS, sarcopenia, kidney diseases and Alzheimer's disease.

In yet another aspect the present invention relates to the composition according to the invention for use in the prevention or slowing of aging and/or of cellular senescence. In yet another aspect the present invention relates to the composition according to the invention for use in the manufacture of a medicament for the prevention or slowing of aging and/or of cellular senescence.

In another aspect the present invention relates to a pharmaceutical composition comprising the composition of the invention, comprising a pharmaceutically acceptable excipient.

In another aspect the present invention relates to a DNA molecule encoding the mRNA comprised in the composition of the invention.

Yet a further aspect of the invention relates to a virus like particle (VLP) comprising a HERV envelope protein as defined according to the invention described herein.

### DETAILED DESCRIPTION

### Definitions

Administering: As used herein, “administering” refers to a method of delivering a composition to a subject or patient. A method of administration may be selected to target delivery (e.g., to specifically deliver) to a specific region or system of a body. For example, an administration may be parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g., by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter. Preferred means of administration are intramuscular, intravenous, intradermal or subcutaneous.

And/or: The term “and/or” wherever used herein includes the meaning of “and”, “or” and “all or any other combination of the elements connected by said term”.

Approximately, about: As used herein, the terms “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a lipid component of an LNP, “about” may mean+/−5% of the recited value. For instance, an LNP including a lipid component having about 40% of a given compound may include 30-50% of the compound.

Comprise: Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

Conjugated: As used herein, the term “conjugated,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. In some embodiments, two or more moieties may be conjugated by direct covalent chemical bonding. In other embodiments, two or more moieties may be conjugated by ionic bonding or hydrogen bonding.

Contacting: As used herein, the term “contacting” means establishing a physical connection between two or more entities. For example, contacting a cell with an mRNA or a lipid nanoparticle composition means that the cell and mRNA or lipid nanoparticle are made to share a physical connection. Methods of contacting cells with external entities both in vivo, in vitro, and ex vivo are well known in the biological arts. In exemplary embodiments of the disclosure, the step of contacting a mammalian cell with a composition (e.g., a transfection agent or a nanoparticle or pharmaceutical composition of the disclosure) is performed in vivo. For example, contacting a lipid nanoparticle composition and a cell (for example, a mammalian cell) which may be disposed within an organism (e.g., a mammal) may be performed by any suitable administration route (e.g., parenteral administration to the organism, including intravenous, intramuscular, intradermal, and subcutaneous administration). For a cell present in vitro, a composition (e.g., a transfection agent, e.g. a lipid nanoparticle) and a cell may be contacted, for example, by adding the composition to the culture medium of the cell and may involve or result in transfection. Moreover, more than one cell may be contacted by a transfection agent or a nanoparticle composition.

Delivering: As used herein, the term “delivering” means providing an entity to a destination. For example, delivering a therapeutic and/or prophylactic to a subject may involve administering a composition including the therapeutic and/or prophylactic to the subject (e.g., by an intravenous, intramuscular, intradermal, or subcutaneous route). Administration of a composition to a mammal or mammalian cell may involve contacting one or more cells with the composition.

Encapsulate: As used herein, the term “encapsulate” means to enclose, surround, or encase. In some embodiments, a compound, polynucleotide (e.g., an mRNA), or other composition may be fully encapsulated, partially encapsulated, or substantially encapsulated. For example, in some embodiments, an mRNA of the disclosure may be encapsulated in a lipid nanoparticle, e.g., a liposome.

Effective amount: The “effective amount” of an agent is that amount sufficient to effect beneficial or desired results, for example, clinical results, and, as such, an “effective amount” depends upon the context in which it is being applied. For example, in the context of the amount of a target cell delivery potentiating lipid in a lipid composition (e.g., LNP) of the disclosure, an effective amount of a target cell delivery potentiating lipid is an amount sufficient to effect a beneficial or desired result as compared to a lipid composition (e.g., LNP) lacking the target cell delivery potentiating lipid. Non-limiting examples of beneficial or desired results effected by the lipid composition (e.g., LNP) include increasing the percentage of cells transfected and/or increasing the level of expression of a protein encoded by a nucleic acid associated with/encapsulated by the lipid composition (e.g., LNP). In the context of administering a target cell delivery potentiating lipid-containing lipid nanoparticle such that an effective amount of lipid nanoparticles are taken up by target cells in a subject, an effective amount of target cell delivery potentiating lipid-containing LNP is an amount sufficient to effect a beneficial or desired result as compared to an LNP lacking the target cell delivery potentiating lipid. Non-limiting examples of beneficial or desired results in the subject include increasing the percentage of cells transfected, increasing the level of expression of a protein encoded by a nucleic acid associated with/encapsulated by the target cell delivery potentiating lipid-containing LNP and/or increasing a prophylactic or therapeutic effect in vivo of a nucleic acid, or its encoded protein, associated with/encapsulated by the target cell delivery potentiating lipid containing LNP, as compared to an LNP lacking the target cell delivery potentiating lipid. In some embodiments, a therapeutically effective amount of target cell delivery potentiating lipid containing LNP is sufficient, when administered to a subject suffering from or susceptible to an infection, disease, disorder, and/or condition, to treat, improve symptoms of, diagnose, prevent, and/or delay the onset of the infection, disease, disorder, and/or condition. In another embodiment, an effective amount of a lipid nanoparticle is sufficient to result in expression of a desired protein in at least about 5%, 10%, 15%, 20%, 25% or more of target cells. For example, an effective amount of target cell delivery potentiating lipid-containing LNP can be an amount that results in transfection of at least 5%, 10%, 15%, 20%, 25%, 30%, or 3 5% of target cells after a single intravenous injection.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Ex vivo: As used herein, the term “ex vivo” refers to events that occur outside of an organism (e.g., animal, plant, or microbe or cell or tissue thereof). Ex vivo events may take place in an environment minimally altered from a natural (e.g., in vivo) environment.

Fragment: A “fragment,” as used herein, refers to a portion. For example, fragments of proteins may include polypeptides obtained by digesting full-length protein isolated from cultured cells or obtained through recombinant DNA techniques. A fragment of a protein can be, for example, a portion of a protein that includes one or more functional domains such that the fragment of the protein retains the functional activity of the protein.

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Kozak Sequence: The term “Kozak sequence” (also referred to as “Kozak consensus sequence”) refers to a translation initiation enhancer element to enhance expression of a gene or open reading frame, and which in eukaryotes, is located in the 5′ UTR. The Kozak consensus sequence was originally defined as the sequence GCCRCC, where R=a purine, following an analysis of the effects of single mutations surrounding the initiation codon (AUG) on translation of the preproinsulin gene (Kozak (1986) Cell 44:283-292). Polynucleotides disclosed herein comprise a Kozak consensus sequence, or a derivative or modification thereof. (Examples of translational enhancer compositions and methods of use thereof, see U.S. Pat. No. 5,807,707 to Andrews et al., incorporated herein by reference in its entirety; U.S. Pat. No. 5,723,332 to Chernajovsky, incorporated herein by reference in its entirety; U.S. Pat. No. 5,891,665 to Wilson, incorporated herein by reference in its entirety.)

Metastasis: As used herein, the term “metastasis” means the process by which cancer spreads from the place at which it first arose as a primary tumor to distant locations in the body. A secondary tumor that arose as a result of this process may be referred to as “a metastasis.”

Modified: As used herein “modified” or “modification” refers to a changed state or a change in composition or structure of a molecule of the disclosure (e.g., polynucleotide, e.g., mRNA). Molecules (e.g., polynucleotides) may be modified in various ways including chemically, structurally, and/or functionally. For example, polynucleotides may be structurally modified by the incorporation of one or more RNA elements, wherein the RNA element comprises a sequence and/or an RNA secondary structure(s) that provides one or more functions (e.g., translational regulatory activity). Accordingly, polynucleotides of the disclosure may be comprised of one or more modifications (e.g., may include one or more chemical, structural, or functional modifications, including any combination thereof). In one embodiment, mRNA molecules of the present disclosure are modified by the introduction of non-natural nucleosides and/or nucleotides, e.g., as it relates to the natural ribonucleotides A, U, G, and C. Noncanonical nucleotides such as the cap structures are not considered “modified” although they differ from the chemical structure of the A, C, G, U ribonucleotides.

mRNA: As used herein, an “mRNA” refers to a messenger ribonucleic acid. An mRNA may be naturally or non-naturally occurring. For example, an mRNA may include modified and/or non-naturally occurring components such as one or more nucleobases, nucleosides, nucleotides, or linkers. An mRNA may include a cap structure, a chain terminating nucleoside, a stem loop, a poly-A sequence, and/or a polyadenylation signal. An mRNA may have a nucleotide sequence encoding a polypeptide. An mRNA may also have a nucleotide sequence encoding multiple (for example at least two or at least three) different polypeptides. Translation of an mRNA, for example, in vivo translation of an mRNA inside a mammalian cell, may produce a polypeptide. Traditionally, the basic components of an mRNA molecule include at least a coding region, a 5′-untranslated region (5′UTR), a 3′UTR, a 5′ cap and a poly-A sequence. In the mRNA sequences disclosed herein in the context of the present invention (in particular in the tables outlined below), the symbol “T” is construed to represent uracil (or a modified uracil such as N1-methylpseudouridine where indicated) in said mRNA.

Nanoparticle: As used herein, “nanoparticle” refers to a particle having any one structural feature on a scale of less than about 1000 nm that exhibits novel properties as compared to a bulk sample of the same material. Routinely, nanoparticles have any one structural feature on a scale of less than about 500 nm, less than about 200 nm, or about 100 nm. Also, routinely, nanoparticles have any one structural feature on a scale of from about 50 nm to about 500 nm, from about 50 nm to about 200 nm or from about 70 to about 120 nm. In exemplary embodiments, a nanoparticle is a particle having one or more dimensions of the order of about 1-1000 nm. In other exemplary embodiments, a nanoparticle is a particle having one or more dimensions of the order of about 10-500 nm. In other exemplary embodiments, a nanoparticle is a particle having one or more dimensions of the order of about 50-200 nm. A spherical nanoparticle would have a diameter, for example, of between about 50-100 or 70-120 nanometers. A nanoparticle most often behaves as a unit in terms of its transport and properties. It is noted that novel properties that differentiate nanoparticles from the corresponding bulk material typically develop at a size scale of under 1000 nm, or at a size of about 100 nm, but nanoparticles can be of a larger size, for example, for particles that are oblong, tubular, and the like. Although the size of most molecules would fit into the above outline, individual molecules are usually not referred to as nanoparticles.

Nucleic acid: As used herein, the term “nucleic acid” is used in its broadest sense and encompasses any compound and/or substance that includes a polymer of nucleotides, unless specified otherwise. These polymers are often referred to as polynucleotides. Comprised is also a chemical derivatization of a nucleic acid on a nucleotide base, on the sugar or on the phosphate, and nucleic acids containing non-natural nucleotides and nucleotide analogs. Exemplary nucleic acids or polynucleotides of the disclosure include, but are not limited to, ribonucleic acids (RNAs), deoxyribonucleic acids (DNAs), DNA-RNA hybrids, RNAi-inducing agents, RNAi agents, siRNAs, shRNAs, miRNAs, antisense RNAs, ribozymes, catalytic DNA, RNAs that induce triple helix formation, threose nucleic acids (TNAs), glycol nucleic acids (GNAs), peptide nucleic acids (PNAs), locked nucleic acids (LN As, including LNA having a ß-D-ribo configuration, a-LNA having an a-L-ribo configuration (a diastereomer of LNA), 2′-amino-LNA having a 2′-amino functionalization, and 2′-amino-a-LNA having a 2′-amino functionalization) or hybrids thereof.

Nucleobase: As used herein, the term “nucleobase” (alternatively “nucleotide base” or “nitrogenous base”) refers to a purine or pyrimidine heterocyclic compound found in nucleic acids, including any derivatives or analogs of the naturally occurring purines and pyrimidines that confer improved properties (e.g., binding affinity, nuclease resistance, chemical stability) to a nucleic acid or a portion or segment thereof. Adenine, cytosine, guanine, thymine, and uracil are the nucleobases predominately found in natural nucleic acids. Other natural, non-natural, and/or synthetic nucleobases, as known in the art and/or described herein, can be incorporated into nucleic acids.

Nucleoside/Nucleotide: As used herein, the term “nucleoside” refers to a compound containing a sugar molecule (e.g., a ribose in RNA or a deoxyribose in DNA), or derivative or analog thereof, covalently linked to a nucleobase (e.g., a purine or pyrimidine), or a derivative or analog thereof (also referred to herein as “nucleobase”), but lacking an internucleoside linking group (e.g., a phosphate group). As used herein, the term “nucleotide” refers to a nucleoside covalently bonded to an internucleoside linking group (e.g., a phosphate group), or any derivative, analog, or modification thereof that confers improved chemical and/or functional properties (e.g., binding affinity, nuclease resistance, chemical stability) to a nucleic acid or a portion or segment thereof.

Open Reading Frame: As used herein, the term “open reading frame”, abbreviated as “ORF”, refers to a segment or region of an mRNA molecule that encodes a polypeptide. The ORF comprises a continuous stretch of non-overlapping, in-frame codons, beginning with the initiation codon and ending with a stop codon, and is translated by the ribosome.

Patient: As used herein, “patient” refers to a subject who may seek or need treatment, requires treatment, is receiving treatment, will receive treatment, or a subject who is under care by a trained professional for a particular disease or condition. In particular embodiments, a patient is a human patient. In some embodiments, a patient is a patient suffering from an autoimmune disease, e.g., as described herein.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipient: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethyl cellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphor sulfonate, citrate, cyclopentane propionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Polyadenyl/poly-A tail/sequence: “Polyadenyl sequence”, “poly-A sequence” or “poly-A tail” refers to an RNA molecule's sequence of adenyl residues typically located at the 3′ end. Such a sequence may be attached during RNA transcription. A poly-A sequence is usually attached to the free 3′ end of the RNA by a template independent RNA polymerase after transcription in the nucleus. Artificially, a poly-A may be attached by transcription from a DNA template containing complementary repeated thymidyl residues. Alternatively, the mRNA as described herein may comprise a polyadenylation signal, which is defined herein as a signal, which conveys polyadenylation to a (transcribed) RNA by specific protein factors. The poly-A sequence is important for the nuclear export, translation, and stability of mRNA and is shortened over time, and eventually leading to enzymatic mRNA degradation

Polypeptide: As used herein, the term “polypeptide” or “polypeptide of interest” refers to a polymer of amino acid residues typically joined by peptide bonds that can be produced naturally (e.g., isolated or purified) or synthetically.

Prevent/preventing: As used herein, “prevent” or “preventing” refers to partially or completely inhibiting the onset of one or more symptoms or features of a particular infection, disease, disorder, and/or condition.

Prophylaxis: As used herein, the term “prophylaxis” refers to partially or completely inhibiting the onset of one or more symptoms or features of a particular infection, disease, disorder, and/or condition. “Prophylactic” is also used in that sense.

RNA: As used herein, an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a poly-A sequence, and/or a polyadenylation signal. An RNA may have a nucleotide sequence encoding a polypeptide of interest. For example, an RNA may be a messenger RNA (mRNA). Translation of an mRNA encoding a particular polypeptide, for example, in vivo translation of an mRNA inside a mammalian cell, may produce the encoded polypeptide.

RNA element: As used herein, the term “RNA element” refers to a portion, fragment, or segment of an RNA molecule that provides a biological function and/or has biological activity (e.g., translational regulatory activity). Modification of a polynucleotide by the incorporation of one or more RNA elements, such as those described herein, provides one or more desirable functional properties to the modified polynucleotide. RNA elements, as described herein, can be naturally-occurring, non-naturally occurring, synthetic, engineered, or any combination thereof. For example, naturally-occurring RNA elements that provide a regulatory activity include elements found throughout the transcriptomes of viruses, prokaryotic and eukaryotic organisms (e.g., humans). RNA elements in particular eukaryotic mRNAs and translated viral RNAs have been shown to be involved in mediating many functions in cells. Exemplary natural RNA elements include, but are not limited to, translation initiation elements (e.g., internal ribosome entry site (IRES), see Kieft et al., (2001) RNA 7(2): 194-206), translation enhancer elements (e.g., the APP mRNA translation enhancer element, see Rogers et al., (1999) J Biol Chem 274(10):6421-6431), mRNA stability elements (e.g., AU-rich elements (AREs), see Garneau et al., (2007) Nat Rev Mol Cell Biol 8(2): 113-126), translational repression element (see e.g., Blumer et al., (2002) Mech Dev 110(1-2):97-112), protein-binding RNA elements (e.g., iron-responsive element, see Selezneva et al., (2013) J Mol Biol 425(18):3301-3310), cytoplasmic polyadenylation elements (Villalba et al., (2011) Curr Opin Genet Dev 21(4):452-457), and catalytic RNA elements (e.g., ribozymes, see Scott et al., (2009) Biochim Biophys Acta 1789(9-10):634-641).

Sequence: The term “sequence” as used herein, should generally be understood to include both the relevant amino acid sequence as well as nucleic acids or nucleotide sequences encoding the same, unless the context requires a more limited interpretation. Amino acid sequences are interpreted to mean a single amino acid or an unbranched sequence of two or more amino acids, depending of the context. Nucleotide sequences are interpreted to mean an unbranched sequence of 3 or more nucleotides.

Specific delivery: As used herein, the term “specific delivery”, “specifically deliver,” or “specifically delivering” means delivery of more (e.g., at least 10% more, at least 20% more, at least 30% more, at least 40% more, at least 50% more, at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a therapeutic and/or prophylactic by a nanoparticle to a target cell of interest (e.g., mammalian target cell) compared to an off-target cell (e.g., non-target cells). The level of delivery of a nanoparticle to a particular cell may be measured by comparing the amount of protein produced in target cells versus non-target cells (e.g., by mean fluorescence intensity using flow cytometry, comparing the fraction (%) of target cells versus non-target cells expressing the protein (e.g., by quantitative flow cytometry), comparing the amount of protein produced in a target cell versus non-target cell to the amount of total protein in said target cells versus non-target cell, or comparing the amount of therapeutic and/or prophylactic in a target cell versus non-target cell to the amount of total therapeutic and/or prophylactic in said target cell versus non-target cell. It will be understood that the ability of a nanoparticle to specifically deliver to a target cell need not be determined in a subject being treated, it may be determined in a surrogate such as an animal model (e.g., a mouse or NHP model).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Therapeutic agent: The term “therapeutic agent” refers to any agent that, when administered to a subject, has a therapeutic, diagnostic, and/or prophylactic effect and/or elicits a desired biological and/or pharmacological effect.

Transfection: As used herein, the term “transfection” refers to methods to introduce a species (e.g., a polynucleotide, such as a mRNA) into a cell.

Subject: As used herein, the term “subject” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants. In some embodiments, a subject may be a patient.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Variant: As used herein, the term “variant” refers to a molecule having at least 50%, 60%, 70%, 80%, 85%, 90%, 95%, 96%, 97%, 98%, 99% or 100% activity of the wild type molecule, e.g., as measured by an art-recognized assay.

Wild type/WT: “Wild type” (or “WT”) refers to the genotype or phenotype of the natural form of a feature or sequence, i.e. the original form of a feature or the unmutated form of a sequence. In particular when mentioned in the context of an immune suppressive domain (ISD), this wild type ISD is active, i.e. it suppresses an immune response. Activity or inactivation of the ISD may be determined as described elsewhere herein.

Percentage identity or % identity: this term refers to a percentage of nucleotides or amino acids, which are identical in an optimal alignment between two nucleotide or amino acid sequences to be compared. Comparisons of two sequences usually requires a step of optimal alignment, which may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 44 3, and with the aid of the similarity search algorithm by Pearson and Lipman, 1988, Proc. Natl Acad. Sei. USA 85, 2444 or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.). Then one or more local regions of the two sequences corresponding to each other are identified. Percentage identity throughout the sequences is then calculated by determining the number of identical positions shared by the two sequences, which is divided by length of the reference sequence. This result is then multiplied by 100. In other words, the percent sequence identity defines the total number of amino acids (when amino acid sequences are compared) or total number of nucleotides (when nucleotide sequences are compared) that are identical in the query sequence over the entire length of the reference sequence. The program “BLAST 2 sequences” is an exemplary tool to perform this calculation, available on the website http://www.ncbi.nlm.nih.gov/blast/bl2seq/wblast2.cgi. 5′/3′ untranslated regions/UTRs: As used herein, “3′” or “3′ end” of a nucleic acid is that end which has a free hydroxy group and “5′” or “5” end“end of a nucleic acid” that end which has a free phosphate group. In a diagrammatic representation of double-stranded nucleic acids, the 3′ end is right-hand and the 5′ end is on the left-hand side:

5′ end 5′--P-NNNNNNN-OH--3′
3′ end 3′--HO-NNNNNNN-P--5′

“Untranslated regions” or “UTRs”, as used herein, refers to either of two sections, one on each side of a coding sequence of mRNA, which are not part of the protein coding region. On the 5′ end the UTR is called 5′ UTR (or leader sequence), on the 3′ side the UTR is called 3′ UTR (or trailer sequence). The UTRs may contain RNA elements regulating translation and/or transcription, as described elsewhere herein. For instance, the 5′ UTR facilitates translation initiation by allowing the ribosome to bind to the sequence and the 3′ UTR is for instance known to be involved in translation termination as well as post-transcriptional modification.

### LNPs Comprising HERV Envelope Protein

In a first aspect the present invention relates to a composition comprising a transfection agent and an mRNA which encodes at least a human endogenous retrovirus (HERV) envelope protein or an immunogenic part thereof; wherein said HERV envelope protein comprises an immune-suppressive domain (ISD); wherein the HERV Env protein comprises a mutated immune-suppressive domain (ISD) that reduces its immune-suppressive property compared to the wildtype ISD.

In one embodiment the invention may relate to the composition of the invention or a pharmaceutically acceptable salt thereof.

### Transfection Agents

Transfection agents of the composition may for instance be any compounds, formulations or mixtures that enhance transport or uptake of a nucleic acid into cells, including cells of different tissues. These agents can increase the uptake of the amount of a nucleic acid, when applied in an effective amount, as defined elsewhere herein. Such effect is caused by one or more substances comprised by the transfection reagent promoting said uptake. The protein or peptide encoded by the introduced nucleic acid can then modulate, evoke or be integrated in cellular processes of the target cell.

Transfection agent compositions may for instance be composed adjusted for the targeted cell type and/or the substance to be delivered, as well as depending on other parameters such as the delivery environment, i.e. in vivo or in vitro. Suitable transfection agents comprise a range of different uptake promoting substances selected from the non-limiting group consisting of: calcium phosphate; cationic polymers, such as DEAE-dextran or polyethylenimine (PEI); liposome forming substances or mixtures thereof, such as cationic lipids like 2,3-dioleoyloxy-N-[2(sperminecarboxamido)ethyl]-N,N-dimethyl-1-propaniminium-trifluoroacetate (DOSPA), Dioleoyl-3-trimethylammonium propane (DOTMA) or dioleoyloxypropyl-trimethylammonium (DOTAP), and/or helper lipids like dioleoyl phosphatidylethanolamine (DOPE), cholesterol and polyethylene glycol (PEG)-lipid; non-liposomal agents, such as for instance FuGENE®, which is commercially available from Promega; and dendrimers. Further preferred is a wide range of commercially available Lipofectamine mixtures (for instance from ThermoFisher Scientific).

Thus, in one embodiment the transfection agent is a transfection agent that comprises a cationic lipid and/or a cationic polymer.

In a preferred embodiment the composition comprises liposomes that comprise a cationic lipid and the mRNA of the invention.

In a more preferred embodiment the transfection agent comprises DOSPA (2,3-dioleoyloxy-N-[2(sperminecarboxamido)ethyl]-N,N-dimethyl-1-propaniminium trifluoroacetate) and/or DOPE (1,2-Dioleoyl-sn-glycerophosphoethanolamine). In a most preferred embodiment the transfection agent comprises DOSPA (2,3-dioleoyloxy-N-[2(sperminecarboxamido)ethyl]-N,N-dimethyl-1-propaniminium trifluoroacetate) and DOPE (1,2-Dioleoyl-sn-glycerophosphoethanolamine), preferably in a molar ratio of 3:1.

### Lipid Nanoparticles (LNPs)

The transfection agent may be a composition particularly suitable for the application of an antigen encoding nucleic acid, e.g. an mRNA, to a subject. Such suitable compositions are known in the art. Such a suitable composition may be a composition of lipids with beneficial properties in vivo, for instance a Lipid Nanoparticle (LNP) composition. LNPs increase circulation time in the body and effectively help to deliver antigen encoding nucleotide sequences, e.g. mRNA, to the target site and have thus emerged as a suitable non-viral encapsulating delivery vehicle for exogenous mRNA.

Thus, in one embodiment, the composition comprises lipid nanoparticles (LNPs) that comprise the mRNA of the invention.

LNPs include various lipid-based platforms such as liposomes, nanostructured lipid carriers (NLCs), and solid lipid nanoparticles (SLNs). Thus, in one embodiment the LNP composition is selected from a liposome composition, nanostructured lipid carrier (NLCs) composition and solid lipid nanoparticle (SLNs) composition.

The composition comprising an mRNA and a transfection agent as described herein may benefit from the use of LNP as a transfection agent, since it has been observed that formulation in LNPs can reduce adverse responses. In preferred embodiments, LNPs are used that comprise lipids known to exhibit a reduced Toll-Like-Receptor (TLR) agonism. It has been observed that reduced activation of TLR signaling plays a key role in triggering RNA vaccine-associated innate signaling and the triggering effect is believed to be amplifiable by certain lipids used in vaccine formulations, which reduce TLR signaling (Tahtinen, S., Tong, A J., Himmels, P. et al. IL-1 and IL-1ra are key regulators of the inflammatory response to RNA vaccines. Nat Immunol 23, 532-542 (2022)). Without wishing to be bound by theory it is predicted that the use of such lipids will increase immunogenicity, leading to a potent innate immune response to the mRNA encoded antigen. Preferred examples include LNPs comprising ionizable lipids. Further preferred examples include LNP compositions comprising the following lipids: DLin-MC3-DMA (MC3), 9-Heptadecanyl 8-{(2-hydroxyethyl) [6-oxo-6-(undecyloxy)hexyl]amino} octanoate (SM-102), (4-hydroxybutyl) azanediyl)bis(hexane-6,1-diyl)bis(2-hexyldecanoate) (ALC-0315), any of the lipids H, M, P, Q, and N in Hasset et al. (2019) (Optimization of Lipid Nanoparticles for Intramuscular Administration of mRNA Vaccines. Mol Ther Nucleic Acids. 2019 Apr. 15; 15:1-11), 1,2-di-O-octadecenyl-3-trimethylammonium propane (DOTMA) and 1,2-dioleoyl-sn-3phosphoethanolamine (DOPE), and DOTMA and DOPE in a ratio of 1:1. Another preferred example of LNP composition comprises at least one lipid selected from the group consisting of (4-hydroxybutyl) azanediyl)bis(hexane-6,1-diyl)bis(2-hexyldecanoate) (ALC-0315), 2-[(polyethylene glycol)-2000]-N,N ditetradecylacetamide (ALC-0159), 1,2-Distearoyl-sn-glycero-3-phosphocholine (DSPC) and Cholesterol. In a more preferred example, an LNP comprises all of the lipids ALC-0315, ALC-0159, DSPC and Cholesterol. In an even more preferred example these lipids are used in a ratio of ALC-0315:ALC-0159:DSPC:Cholesterol of 46.3:9.4:42.7:1.6.

Further aspects (i), (ii), (iii) and (iv) of the invention provide the following:
(i) an LNP comprising an mRNA of the invention;
(ii) a liposome comprising an mRNA of the invention;
(iii) a polyplex comprising an mRNA of the invention;
(iv) a lipoplex comprising an mRNA of the invention.

Yet a further aspect of the invention relates to a virus like particle (VLP) comprising a HERV envelope protein as defined according to the invention described herein. Preferably, the envelope protein comprises a mutated ISD with a mutation selected from the group consisting of L522A, A523Q, N524A, Q525A, I526A, N527A, D528A, L529A, R530A, Q531A, T532A, V533A, I534A and W535A, preferably wherein the mutated ISD comprises a sequence according to any one of SEQ ID NO:s 4 or 8-20 (most preferably said mutated ISD comprises or consists of sequence LANAINDLRQTVIW (SEQ ID NO: 4)) and wherein the HERV envelope protein further comprises the amino acid sequence:

| G | C | L | M | P | A | V | Q | N | W | L |
|---|---|---|---|---|---|---|---|---|---|---|
| 170 | 171 | 172 | 173 | 174 | 175 | 176 | 177 | 178 | 179 | 180 |

| V | E | V | P | T | V | S | P | I | S |
|---|---|---|---|---|---|---|---|---|---|
| 181 | 182 | 183 | 184 | 185 | 186 | 187 | 188 | 189 | 190 |
| R | F | T | Y | H | M | V | S | G | M |
| 191 | 192 | 193 | 194 | 195 | 196 | 197 | 198 | 199 | 200 |
| S | L | R | P | R | V | N | Y | L | Q |
| 201 | 202 | 203 | 204 | 205 | 206 | 207 | 208 | 209 | 210 |

wherein at least one amino acid at a position in the range of from positions 170 to 210 has been replaced by a cysteine; or
wherein the HERV envelope protein comprises a cysteine at position 190, and more preferably wherein the HERV envelope protein comprises the amino acid mutation S190C; and/or wherein the HERV envelope protein comprises the sequence VQNWLVEVPTVSPICRFTYHMVSGMSLRP;
and/or wherein the HERV envelope protein comprises an amino acid sequence having at least 90% sequence identity with SEQ ID NO: 2, wherein the HERV envelope protein comprises a cysteine at a position within said HERV envelope protein that corresponds to amino acid 190 of SEQ ID NO: 2.

### Lipid Content for Encapsulation

Encapsulation of RNA is achieved by combining the RNA with lipids at an acidic pH at which an ionizable lipid is positively charged, thus ensuring a charge-driven interaction with the negatively charged nucleic acid. Later, pH is adjusted above the ionizable lipid's pKa resulting in approximately neutral surface charge which is suitable for clinical administration. A multitude of suitable components of LNP compositions for the delivery of nucleic acids, such as mRNAs, are known in the art and will be clear to a skilled person. More general information and a multitude of examples concerning lipid nanoparticle formulations are provided by the art, for instance in publications by Barba et al. (2019), Schoenmaker et al (2021) or MacLachlan (2007) (Barba et al., 2019, Lipid Delivery Systems for Nucleic-Acid-Based-Drugs: From Production to Clinical Applications. Pharmaceutics; 11(8):360; Schoenmaker et al., 2021; mRNA-lipid nanoparticle COVID-19 vaccines: Structure and stability. Int J Pharm.; 601:120586; MacLachlan, 2007, Liposomal formulations for nucleic acid delivery. In Antisense Drug Technology; 255-288). Thus, the embodiments set forth below are examples only and are in no way limiting.

In one embodiment, the LNP composition may comprise one or more ionizable lipids. The one or more ionizable lipids may preferably comprise cationic lipids. Unless a different meaning is clear from the specific context, the term “cationic” means that the respective structure bears a positive charge, either permanently, or not permanently but in response to certain conditions such as pH. Thus, the term “cationic” covers both “permanently cationic” and “cationisable”. Suitable cationic lipids of the composition of the present invention are known in the art, and may for instance advantageously be selected from the non-limiting group consisting of N,N-dioleyl-N,N-dimethylammonium chloride (DODAC), N-(2,3-dioleyloxy)propyl)N, N,N-trimethylammonium chloride (DOTMA); N,N-distearyl-N,N-dimethylammonium bromide (DDAB); N-(2,3dioleoyloxy)propyl)-N,N,N-trimethylammonium chloride (DOTAP); 3-(N-(N′,N′dimethylaminoethane)-carbamoyl) cholesterol (DC-Chol), N-(1-(2,3-dioleoyloxy)propyl)N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy) propylamine (DODMA), and N-(1,2dimyristyloxyprop-3-yl)-N,N-dimethyl-N-hydroxyethyl ammonium bromide (DMRIE). Additionally, suitable cationic lipids of the composition of the present invention may for instance be selected from the non-limiting group of commercial preparations of cationic lipids consisting of LIPOFECTIN® (commercially available cationic liposomes comprising DOTMA and 1,2-dioleoyl-sn-3phosphoethanolamine (DOPE), from GIBCO/BRL, Grand Island, N.Y.); LIPOFECTAMINE® (commercially available cationic liposomes comprising N-(1-(2,3dioleyloxy)propyl)-N-(2-(sperminecarboxamido)ethyl)-N,N-dimethylammonium trifluoroacetate (DOSPA) and DOPE, from GIBCO/BRL); and TRANSFECTAM® (commercially available cationic lipids comprising dioctadecylamidoglycyl carboxyspermine (DOGS) in ethanol from Promega Corp., Madison, Wis.).

The ionizable lipids of the disclosure may comprise a central amine moiety and at least one biodegradable group. Thus, in an even more preferred embodiment, the ionizable lipid may be an ionizable cationic amino lipid.

In some embodiments, the lipid-based composition (e.g., LNP) described herein comprises one or more non-cationic helper lipids. As used herein, the term “non-cationic helper lipid” refers to a lipid comprising at least one fatty acid chain of at least 8 carbons in length and at least one polar head group moiety. In some embodiments, the non-cationic helper lipid may be a phospholipid. In general, phospholipids comprise a phospholipid moiety and one or more fatty acid moieties. A phospholipid moiety may be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. A fatty acid moiety may be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid. Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipids, such as sphingomyelin.

In a preferred embodiment, the helper lipid or phospholipid may be neutral. The LNP composition may, for instance, also comprise a DSPC analog, a DSPC substitute, oleic acid, or an oleic acid analog, a non-phosphatidyl choline (PC) zwitterionic lipid, a DSPC analog, oleic acid, an oleic acid analog, or a 1,2-distearoyl-i77-glycero-3-phosphocholine (DSPC) substitute. In an even more preferred embodiment, the helper lipid or phospholipid of the LNP composition may be DSPC.

In some embodiments the lipid-based composition (e.g., LNP) may comprise one or more structural lipids. In a preferred embodiment the structural lipid may be a sterol. Such sterols may include cholesterol, ß-sitosterol, sitostanol, camesterol, stigmasterol, and brassicasterol. In an even more preferred embodiment said sterol may preferably be cholesterol.

In some embodiments the LNP composition may comprise another lipid, which is a phospholipid substitute or replacement. The phospholipid or phospholipid substitute or replacement may be, for example, one or more saturated or (poly)unsaturated phospholipids, or phospholipid substitutes, or a combination thereof. In a preferred embodiment such a phospholipid substitute or replacement may be a pegylated or PEG lipid. The incorporation of a pegylated lipid leads for instance to steric stabilization of the nanoparticle core shell. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. In some embodiments, the PEG-lipid includes, but is not limited to, PEG-disteryl glycerol (PEG-DSG), or PEG-1,2-dimyristyloxlpropyl-3-amine (PEG-c-DMA), 1,2-dimyristoyl-snglycerol methoxypolyethylene glycol (PEG-DMG), PEG-dipalmetoleyl, PEG-dioleyl, PEG-distearyl, PEG-diacylglycamide (PEG-DAG), PEG-DLPE, PEG-DMPE, PEG-dipalmitoyl phosphatidylethanolamine or -choline (PEG-DPPE, PEG-DPPC), 1,2-distearoyl-sn-glycero-3-phosphoethanolamine-N-amino polyethyleneglycol (PEG-DSPE) lipid. In a preferred embodiment the LNP composition may comprise PEG-DMG.

In one embodiment the LNP composition comprises at least one lipid selected from the group consisting of (i) an ionizable lipid, preferably an ionizable cationic lipid, more preferably an ionizable cationic amino lipid; (ii) a non-cationic helper lipid or phospholipid, wherein the lipid is preferably neutral, more preferably wherein the lipid is DSPC; (iii) a sterol or other structural lipid, wherein the sterol is preferably cholesterol; and (iv) a PEG lipid, preferably PEG-DMG.

The lipid components of the LNP composition may be used in suitable molar ratios with respect to the other lipids. The amount of ionizable lipid, preferably cationic lipid, preferably cationic amino-lipid, may range for instance from about 45 mol % to about 50 mol %. The amount of non-cationic helper lipid or phospholipid, preferably a neutral lipid, preferably DSPC, may for instance range from about 5 mol % to about 15 mol %. The amount of structural lipid, such as sterol, preferably cholesterol, may for instance range from about 30 mol % to about 45 mol %. The amount of PEG-lipid in the lipid composition of a pharmaceutical composition disclosed herein may range for instance from about 0.1 mol % to about 5 mol %.

Thus, in one embodiment the composition of the invention, wherein the transfection agent is a lipid nanoparticle (LNP) composition, the LNP composition comprises a molar ratio of about 45 mol % to about 50 mol % ionizable lipid, about 5 mol % to about 15 mol % phospholipid, about 30 mol % to about 45 mol % sterol, and about 1 mol % to about 5 mol % PEG lipid. In a preferred embodiment, the LNP may for instance comprise a molar ratio of 50 mol % ionizable lipid, 10 mol % phospholipid, 38.5 mol % sterol, and 1.5 mol % PEG lipid. In an alternative preferred embodiment, the LNP may for instance comprise a molar ratio of 46.3 mol % ionizable lipid, 9.4 mol % phospholipid, 42.7 mol % sterol, and 1.6 mol % PEG lipid.

In another embodiment, the lipid nanoparticle composition may comprise a targeting moiety, which is a compound or agent that may target nanoparticles to a particular cell, tissue, and/or organ type. Thus, in one embodiment the lipid nanoparticle composition comprising a targeting moiety has the ability to specifically deliver to a particular target cell, tissue, and/or organ type.

It may be desired or useful to further increase the efficacy or potency of the composition of the invention, particularly for instance when the composition comprising the RNA of the invention is applied to a subject or patient for prophylaxis or treatment. Thus, in one embodiment the composition of the invention further comprises an adjuvant. In a preferred embodiment the adjuvant may be a cytokine and more preferably a cytokine selected from the group consisting of INFγ, IL-2, IL-12, GM-CSF, IL-15, and IL-7. The adjuvant may also be introduced as a polynucleotide configured to express one or more of the aforementioned cytokines in eukaryotic cells.

Adjuvants may act by a combination of various mechanisms to elicit and boost immune responses, including one or more of: a sustained release of antigen at the site of injection (depot effect), an up-regulation of (further) cytokines and chemokines, cellular recruitment at the site of administration of the composition, an increased antigen uptake and presentation to antigen presenting cells (APCs), an activation and maturation of APCs, increased expression of major histocompatibility complex (MHC) class II and co-stimulatory molecules, promoting antigen transport to draining lymph nodes and activation of the inflammasome.

mRNA Features

In one embodiment the mRNA comprised by the composition of the invention comprises at least 300 nucleotides, such as preferably at least 400, 500, 800, 1000, 1500, 2000, more preferably at least 3000 or most preferably at least 4000 nucleotides.

Modifying mRNA elements, such as the 5′ cap, 5′- and 3′-untranslated regions (UTRs), the coding region, and polyadenylation tail, helps to reduce excessive mRNA immunogenicity and/or to improve mRNA stability and translational efficiency. Therefore, the mRNA comprised by the composition of the invention may have certain functional sequence features, optimizing its properties with regard to stability, expression efficiency and tolerance in a patient. mRNA molecules may comprise an elongated oligo-A sequence or poly-A sequence at their 3′-end, i.e. a poly-A tail. The 3′ poly-A facilitates nuclear export, and provides RNA stability and translational efficiency of the mRNA. Over time, the poly-A is shortened, eventually leading to the initiation of enzymatic mRNA degradation. Elongating the poly-A may therefore provide additional stability. Thus, in one embodiment the composition comprises an mRNA which comprises at least 60 adenosine nucleotides at the 3′-UTR. In a preferred embodiment, the mRNA comprises at least 100, more preferably 120 adenosine nucleotides at the 3′-UTR.

According to a further embodiment, the mRNA of the invention may comprise a poly(C) (poly-cysteine) tail at the 3′-terminus of typically about 10 to 200 cytosine nucleotides, preferably about 10 to 100 cytosine nucleotides, more preferably about 20 to 70 or even more preferably about 20 to 60 cytosine nucleotides.

Codon optimization is another approach to improve gene expression by changing synonymous codons based on an organism's codon bias. Mutations are introduced into a gene of interest based on a host organism's own codon usage bias to increase translational efficiency in said organism and thus protein expression without altering the sequence of the protein. Thus, in another embodiment the mRNA is codon optimized for expression in a human. Human codon usage is known in the art, as for instance provided by GenScript Codon Usage Frequency Table(chart) Tool. The mRNA of the invention may also be codon optimized for expression in other animals, such as for instance other mammals.

In some cases, modified nucleobases in nucleic acids are introduced into nucleic acid sequences (e.g., RNA nucleic acids, such as mRNA nucleic acids) to improve stability. In one embodiment, the mRNA of the invention may comprise at least one artificially modified nucleotide. In one embodiment the mRNA of the invention comprises no artificially modified nucleotides.

In one embodiment, the mRNA of the invention may have a modified and thus stabilized, by modifying the guanosine/cytosine (G/C) content of the mRNA sequence, particularly increased, compared to the G/C content of the coding region of the respective wild type mRNA, i.e. the unmodified mRNA. According to a specific embodiment at least 5%, 10%, 20%, 30%, 40%, 50%, 60%, more preferably at least 70%, even more preferably at least 80% and most preferably at least 90%, 95% or even 100% of the substitutable codons in the coding region of the mRNA of the invention or the whole sequence of the wild type mRNA sequence may be substituted, thereby increasing the GC/content of said sequence.

In one embodiment, the mRNA of the invention may be modified by modifying, preferably increasing, the cytosine (C) content of the mRNA sequence, preferably of the coding region of the mRNA, compared to the C content of the coding region of the respective wild type mRNA, i.e. the unmodified mRNA. Preferably, the mRNA sequence may be modified such that at least 10%, 20%, 30%, 40%, 50%, 60%, 70% or 80%, or at least 90% of the theoretically possible maximum cytosine-content or even a maximum cytosine-content is achieved.

In some embodiments, the mRNA of the invention may, for instance and without limitation, include at least one of the sequence elements selected from the group consisting of CAP analog structures; suitable promoters or subgenomic promoters yielding a high translation rate; self-amplifying mRNA features (e.g. the mRNA of the invention may be a self-replicating mRNA), such as for instance cytomegalovirus promoter, T7 promoter or subgenomic SFV promoter; Kozak consensus sequence (5′-CCACCATGG-3′); a spacer of 3 to 6 nucleotides between (T7) promoter sequence and Kozak sequence, if present; stabilizing and/or structural sequence elements in UTR sequences. Suitable RNA elements are described in the art and will be clear to a person of average skill in the relevant field.

For instance, suitable CAP analog structures for the mRNA of the invention may be selected from the non-limiting group consisting of Vaccinia 2′-O-Methyltransferase Cap 1, ARCA anti-reverse CAP analogue or β-S-ARCA cap, modified ARCA (e.g. phosphothioate modified ARCA); m7GpppN, cap1 (methylation of the ribose of the adjacent nucleotide of m7G), cap2 (additional methylation of the ribose of the 2nd nucleotide downstream of the m7G), cap3 (additional methylation of the ribose of the 3rd nucleotide downstream of the m7G), cap4 (methylation of the ribose of the 4th nucleotide downstream of the m7G), inosine, N1-methyl-guanosine, 2′-fluoroguanosine, 7-deaza-guanosine, 8-oxo-guanosine, 2-aminoguanosine, LNA-guanosine, and 2-azido-guanosine.

For instance, suitable stabilizing and/or structural sequence elements in UTR sequences for the mRNA of the invention may be based on a variant of the UTR sequence(s) of a gene, such as on a variant of the UTR(s) of an albumin gene, an a-globin gene, a ß-globin gene, a tyrosine hydroxylase gene, a lipoxygenase gene, or collagen alpha gene, such as a collagen alpha 1 gene, or a part thereof. Thus, in some preferred embodiments, the mRNA of the invention may comprise a 5′UTR HBA1, 5′UTR SFV, or 5′UTR 7, 3′UTR HBB; and/or a 3′UTR AES mtRNR1. In another embodiment, an mRNA 5′-UTR may comprise or consist of a nucleic acid sequence, which is derived from the 5′-UTR of a ribosomal protein Large gene or from the 5′-UTR of a vertebrate TOP gene.

The mRNA of the invention, which encodes a HERV envelope protein or an immunogenic part thereof, may also encode said protein as part of a virus like particle (VLP). VLPs are molecules that closely resemble viruses made up of one or more different molecules with the ability to self-assemble and mimic release, form and size of a virus particle but lacking the genetic material for infecting a host cell. In the context of the present invention, they may be formed by encoding a viral Gag protein along with the HERV envelope protein. The gag gene is then translated into a polyprotein, which mediates the formation of the VLP in the absence of other viral proteins, incorporating HERV Env on the VLP's surface.

Thus, in one embodiment the composition of the invention comprises an mRNA, which encodes at least a HERV envelope protein or an immunogenic part thereof with a mutated ISD of the invention and a gag protein. In a preferred embodiment the gag protein may be selected from the same or a different virus as the Env protein.

Further applicable and potentially beneficial features of VLPs to be encoded by the mRNA will be clear to a skilled person of the relevant technical field. For instance, in one embodiment, the mRNA of the invention may encode at least a human endogenous retrovirus (HERV) envelope protein or an immunogenic part thereof with a mutated ISD, a gag protein and a 2A peptide. Furthermore, the Env protein may for instance be encoded to comprise a Surface Unit (SU, also referred to as gp70), a cleavage site, and/or a transmembrane unit (TM, also referred to as p15E). In addition, the transmembrane unit (TM, also referred to as p15E) may for instance comprise a fusion peptide, a transmembrane anchor, and/or a cytoplasmic tail.

In a most preferred embodiment, the mRNA or mRNA construct of the invention, encoding at least a HERV envelope protein or an immunogenic part thereof, may also encode a Gag protein and may comprise a 5′UTR HBA, a 3′UTR HBB and a PolyA, comprising 70 adenine nucleotides. Thus, the mRNA of the invention may have the structure: (5′UTR) HBA1-HERV-K Gag Env ISDmut-(3′UTR) HBB-PolyA(70). In another preferred embodiment, the mRNA or mRNA construct of the invention, encoding at least a HERV envelope protein or an immunogenic part thereof, may also encode a Gag protein and may comprise a 5′UTR of HBA1, a 3′UTR of AES mtRNR1 and a PolyA comprising 100 adenine nucleotides wherein 30 and 70 adenine nucleotides are preferably interrupted by a linker (30-linker-70). Thus, the mRNA of the invention may have the structure: 5′UTR of HBA1-HERV-K Gag Env ISDmut (preferably with ISD mutation Q525A)-3′UTR of AES mtRNR1-PolyA (preferably the poly(A) being A30-linker-A70).

In one aspect the present invention relates to a DNA molecule encoding the mRNA comprised in the composition of the invention.

### HERV Envelope Protein

The present invention provides a platform for displaying antigens to a body's immune system. Thus, in principle the coding for any type of protein, against which it is desired to raise an immune response, can be incorporated in the mRNA construct. In an aspect of the invention the mRNA encoded protein is endogenous retrovirus envelope protein (ERV Env) or an immunogenic protein derived from such proteins. It is generally believed that the vaccine directs ERV Env to dendritic cells (DCs), which present antigens to cells of the adaptive immune system. Presentation on MHC class I induces activation and proliferation of CD8+ T cells. These cytotoxic T lymphocytes (CTLs), specific for antigens of ERV Env, infiltrate tumors and kill cells displaying the respective antigen. Presentation of antigens on MHC class II by professional antigen presenting cells (APCs) activates CD4+ T cells, which subsequently co-activate B cells. Activated B cells that encounter the ERV Env target protein in the circulation or antigens displayed on cells or VLPs release antibodies specific for ERV Env. These antibodies are able to bind their target on cancer cells, inducing destruction and phagocytosis of the malignant cells. In this way, ERV-specific antibodies are able to prevent tumor growth and metastasis. The regained immunogenicity of tumor cells enables priming of a set of diverse tumor-specific T cells recognizing different tumor-associated and tumor-specific antigens. The newly primed and expanded CTLs infiltrate the tumor and kill malignant cells.

Retroviruses are protein-enveloped and thus, retroviral genomes encode Env (envelope protein) as one of the three major proteins. ERVs are the evidence of ancient infections with retroviruses in distant ancestors. Therefore, using envelope proteins as antigens in vaccinations is also useful for targeting a wide range of ERVs. While the present composition in principle may be used for immunizing a number of mammal species, the ERV protein in an aspect of the invention is a human endogenous retrovirus (HERV) protein or an immunogenic part thereof. It has been estimated that every human genome consists of about 8% endogenous retroviral DNA. However, most of the endogenous retroviral DNA is just relics of the former retrovirus. Upon infection, viral RNA was reverse transcribed into proviral DNA, which was integrated into the host genome. Eventually, the provirus was integrated into cells of the germ line and became inheritable, giving rise to endogenous retroviruses. Over millions of years the viral DNA was passed down generations and became fixed in the populations. It follows that a large part of the human genome potentially may be used as antigen-coding part of mRNA like that of the invention.

In one embodiment the HERV is selected from the group consisting of HERV-K, HERV-H, HERV-W, HERV-FRD, HERV-E, HERV-9, HERV-FC, HERV-T, HERV-3, HERV-V1 and HERV-V2. More specifically, the HERV-K, which has been known to be upregulated in prostate cancer, breast cancer, ovarian cancer, lymphomas, melanomas, leukemia and sarcomas, may in one embodiment be selected among the group consisting of HERV-K108 (=ERVK-6), ERVK-19, HERV-K115 (=ERVK-8), ERVK-9, HERV-K113, ERVK-21, ERVK-25, HERV-K102 (=ERVK-7), HERV-K101 (=ERVK-24), HERV-K110 (=ERVK-18); HERV-K. HERV-H may for instance be selected among the group consisting of HERV-H19 (=HERV-H_2q24.3), HERV-H_2q24.1; HERV-W may for instance be selected as ERVW-1 (=Syncytin-1); and HERV-FRD may for instance be selected as ERVFRD-1 (=Syncytin-2).

In another embodiment the HERV is selected from the group consisting of HERV-9, HERV-FC, HERV-T, HERV-E, HERV-3, HERV-V1 and HERV-V2.

For an mRNA encoded HERV-9 envelope protein a preferred mutated immune-suppressive domain (ISD) that reduces its immune-suppressive property compared to the wildtype ISD comprises or consists of the sequence LQNCZGLDLLTAEKGGLCTFLGE (SEQ ID NO: 38).

For an mRNA encoded HERV-FC envelope protein a preferred wildtype immune-suppressive domain (ISD) comprises or consists of the sequence AQNRRALDLLTADKGGTCLFLGE (SEQ ID NO: 39)

For an mRNA encoded HERV-T envelope protein a preferred wildtype immune-suppressive domain (ISD) comprises or consists of the sequence LQNRRGLDLLFLSQGGLCAALGE (SEQ ID NO: 40).

For an mRNA encoded HERV-E envelope protein a preferred wildtype immune-suppressive domain (ISD) comprises or consists of the sequence YQNRLALDYLLAAEGGVCGKFNL (SEQ ID NO: 41).

For an mRNA encoded HERV-3 envelope protein a preferred wildtype immune-suppressive domain (ISD) comprises or consists of the sequence YQNRLALDYLLAQEGGVCGKFNL (SEQ ID NO: 42).

For an mRNA encoded HERV-V1 envelope protein a preferred wildtype immune-suppressive domain (ISD) comprises or consists of the sequence MNNRLALDYLLAEQGGVCAVISK (SEQ ID NO: 43).

For an mRNA encoded HERV-V2 envelope protein a preferred wildtype immune-suppressive domain (ISD) comprises or consists of the sequence MDNRLALDYLLAEQGGVCAVINK (SEQ ID NO: 44).

Other gamma-retroviral ISD sequences of other HERVs can be identified by multiple sequence alignment.

For an mRNA encoded HERV-FC envelope protein, the immune-suppressive domain (ISD) comprising or consisting of the sequence AQNRRALDLLTADKGGTCLFLGE (SEQ ID NO: 39) already comprises mutations rendering the ISD less active or inactive, i.e. the sequence AQNRRALDLLTADKGGTCLFLGE (SEQ ID NO: 39) may be directly used in an HERV-FC envelope protein encoded by an mRNA as described herein.

An active gamma-retroviral ISD (consisting of 23 amino acid positions) may be suitably mutated to a non-immune suppressive ISD by replacing an acidic amino acid residue at position 14 with a different amino acid, such as preferably a basic amino acid residue. Additionally inserting an aromatic amino acid residue at position 20 of the ISD (consisting of 23 amino acid positions) may further improve stability.

In one embodiment the HERV is selected from the group consisting of HERV-9, HERV-T, HERV-E, HERV-3, HERV-V1 and HERV-V2;

wherein the mutated immune-suppressive domain (ISD) that reduces its immune-suppressive property compared to the wildtype ISD comprises or consists of one of the following sequences consisting of 23 amino acid positions: LQNCZGLDLLTAEKGGLCTFLGE (SEQ ID NO: 38) for HERV-9, LQNRRGLDLLFLSQGGLCAALGE (SEQ ID NO: 40) for HERV-T, YQNRLALDYLLAAEGGVCGKFNL (SEQ ID NO: 41) for HERV-E, YQNRLALDYLLAQEGGVCGKFNL (SEQ ID NO: 42) for HERV-3, MNNRLALDYLLAEQGGVCAVISK (SEQ ID NO: 43) for HERV-V1 and MDNRLALDYLLAEQGGVCAVINK (SEQ ID NO: 44) for HERV-V2, wherein one or more amino acids are replaced with a different amino acid. In a preferred embodiment a single amino acid at position 14 is replaced with a different amino acid. In an even more preferred embodiment the single amino acid at position 14 is replaced with a basic amino acid. In an even more preferred embodiment the single amino acid at position 14 is replaced with R.

In a preferred embodiment the HERV is selected from the group consisting of HERV-9, HERV-T, HERV-E, HERV-3, HERV-V1 and HERV-V2, wherein the mutated immune-suppressive domain (ISD) that reduces its immune-suppressive property compared to the wildtype ISD comprises or consists of one of the following sequences: LQNCZGLDLLTAERGGLCTFLGE (SEQ ID NO: 46) for HERV-9, LQNRRGLDLLFLSRGGLCAFLGE (SEQ ID NO: 47) for HERV-T, YQNRLALDYLLAARGGVCGFFNL (SEQ ID NO: 48) for HERV-E, YQNRLALDYLLAQRGGVCGFFNL (SEQ ID NO: 49) for HERV-3, MNNRLALDYLLAERGGVCAFISK (SEQ ID NO: 50) for HERV-V1 and MDNRLALDYLLAERGGVCAFINK (SEQ ID NO: 51) for HERV-V2,

In a preferred embodiment the HERV is selected from the group consisting of HERV-9, HERV-T, HERV-E, HERV-3, HERV-V1 and HERV-V2, wherein the mutated immune-suppressive domain (ISD) that reduces its immune-suppressive property compared to the wildtype ISD comprises or consists of one of the following sequences consisting of 23 amino acid positions: LQNCZGLDLLTAEKGGLCTFLGE (SEQ ID NO: 38) for HERV-9, LQNRRGLDLLFLSQGGLCAALGE (SEQ ID NO: 40) for HERV-T, YQNRLALDYLLAAEGGVCGKFNL (SEQ ID NO: 41) for HERV-E, YQNRLALDYLLAQEGGVCGKFNL (SEQ ID NO: 42) for HERV-3, MNNRLALDYLLAEQGGVCAVISK (SEQ ID NO: 43) for HERV-V1 and MDNRLALDYLLAEQGGVCAVINK (SEQ ID NO: 44) for HERV-V2, wherein a single amino acid at position 14 is replaced with a different amino acid, and wherein a single amino acid at position 20 is replaced with a different amino acid, preferably wherein position is exchanged to increase stability.

In a further preferred embodiment, the mutated immune-suppressive domain (ISD) that reduces its immune-suppressive property compared to the wildtype ISD comprises or consists of one of the following sequences consisting of 23 amino acid positions: LQNCZGLDLLTAEKGGLCTFLGE (SEQ ID NO: 38) for HERV-9, LQNRRGLDLLFLSQGGLCAALGE (SEQ ID NO: 40) for HERV-T, YQNRLALDYLLAAEGGVCGKFNL (SEQ ID NO: 41) for HERV-E, YQNRLALDYLLAQEGGVCGKFNL (SEQ ID NO: 42) for HERV-3, MNNRLALDYLLAEQGGVCAVISK (SEQ ID NO: 43) for HERV-V1 and MDNRLALDYLLAEQGGVCAVINK (SEQ ID NO: 44) for HERV-V2, wherein a single amino acid at position 14 is replaced with R, and wherein a single amino acid at position 20 is replaced with F, preferably wherein position 20 is exchanged to increase stability.

In one embodiment the invention relates to a composition comprising a transfection agent and an mRNA which encodes at least a human endogenous retrovirus (HERV) envelope (Env) protein or an immunogenic part thereof; wherein said HERV envelope protein comprises an immune-suppressive domain (ISD); wherein the HERV Env protein comprises a mutated immune-suppressive domain (ISD) that reduces its immune-suppressive property compared to the wildtype ISD, wherein the HERV selected from the group consisting of HERV-9, HERV-FC, HERV-T, HERV-E, HERV-3, HERV-V1 and HERV-V2;

wherein the mutated immune-suppressive domain (ISD) that reduces its immune-suppressive property compared to the wildtype ISD comprises or consists of one of the following sequences consisting of 23 amino acid positions: LQNCZGLDLLTAEKGGLCTFLGE (SEQ ID NO: 38) for HERV-9; AQNRRALDLLTADKGGTCLFLGE (SEQ ID NO: 39) for HERV-FC, LQNRRGLDLLFLSQGGLCAALGE (SEQ ID NO: 40) for HERV-T, YQNRLALDYLLAAEGGVCGKFNL (SEQ ID NO: 41) for HERV-E, YQNRLALDYLLAQEGGVCGKFNL (SEQ ID NO: 42) for HERV-3 MNNRLALDYLLAEQGGVCAVISK (SEQ ID NO: 43) for HERV-V1 and MDNRLALDYLLAEQGGVCAVINK (SEQ ID NO: 44) for HERV-V2, wherein in SEQ ID NO: 38, 40, 41, 42, 43 and 44 a single amino acid at position 14 is replaced with a different amino acid, and preferably replaced with R, and preferably wherein a single amino acid at position 20 is replaced with a different amino acid, and preferably replaced with F.

In another embodiment the mRNA encoded HERV envelope protein comprises an amino acid sequence having at least 85%, 90%, 95%, 96%, 97%, 98%, 99% or 100% identity to the amino acid sequence of SEQ ID NO: 2 over the entire length of SEQ ID NO:2 (see Table 1).

In one embodiment the HERV is HERV-K. In some embodiments it is preferred that the HERV-K Env protein has a HERV-K Env consensus sequence, more preferably a codon-optimized consensus sequence. A particularly preferred amino acid sequence of wild-type HERV-K Env (without ISD mutation) is shown below and is designated as SEQ ID NO: 1 (see also Table 1):

MNPSEMQRKAPPRRRRHRNRAPLTHKMNKMVTSEEQMKLPSTKKA
EPPTWAQLKKLTQLATKYLENTKVTQTPESMLLAALMIVSMVVSL
PMPAGAAAANYTYWAYVPFPPLIRAVTWMDNPIEVYVNDSVWVPG
PIDDRCPAKPEEEGMMINISIGYRYPPICLGRAPGCLMPAVQNWL
VEVPTVSPISRFTYHMVSGMSLRPRVNYLQDFSYQRSLKFRPKGK
PCPKEIPKESKNTEVLVWEECVANSAVILQNNEFGTIIDWAPRGQ
FYHNCSGQTQSCPSAQVSPAVDSDLTESLDKHKHKKLQSFYPWEW
GEKGISTPRPKIVSPVSGPEHPELWRLTVASHHIRIWSGNQTLET
RDRKPFYTVDLNSSLTVPLQSCVKPPYMLVVGNIVIKPDSQTITC
ENCRLLTCIDSTFNWQHRILLVRAREGVWIPVSMDRPWEASPSVH
ILTEVLKGVLNRSKRFIFTLIAVIMGLIAVTATAAVAGVALHSSV
QSVNFVNDWQKNSTRLWNSQSSIDQKLANQINDLRQTVIWMGDRL
MSLEHRFQLQCDWNTSDFCITPQIYNESEHHWDMVRRHLQGREDN
LTLDISKLKEQIFEASKAHLNLVPGTEAIAGVADGLANLNPVTWV
KTIGSTTIINLILILVCLFCLLLVCRCTQQLRRDSDHRERAMMTM
AVLSKRKGGNVGKSKRDQIVTVSV.

The mRNA encoding the HERV Env protein is preferably constructed so as to allow the encoded protein to be expressed in vivo and presented to the immune system to elicit an immunological response.

Surprisingly, it was found that by replacing one or more amino acid positions of the HERV Env protein with a cysteine, and preferably by replacing serine at position 190 of the HERV Env protein with a cysteine (S190C), and/or by increasing the cysteine content in general, preferably to an even number of cysteines in the HERV Env protein, the expression efficiency of the HERV Env protein from encoding nucleic acid molecules was increased (see FIG. 4), possibly through an increase of stability of the (transcribed) mRNA and/or of translated protein product. For instance, a serine at position 190 of the HERV Env protein may have a destabilizing effect, whereas replacing this position with a cysteine to obtain an even number of 18 cysteines in the HERV Env protein may have a stabilizing effect.

Thus, in one embodiment the HERV envelope protein comprises an even number of cysteines. In another embodiment the HERV envelope protein alternatively or additionally comprises at least 18 cysteines. In another embodiment the HERV envelope protein comprises the amino acid sequence

| G | C | L | M | P | A | V | Q | N | W | L |
|---|---|---|---|---|---|---|---|---|---|---|
| 170 | 171 | 172 | 173 | 174 | 175 | 176 | 177 | 178 | 179 | 180 |

| V | E | V | P | T | V | S | P | I | S |
|---|---|---|---|---|---|---|---|---|---|
| 181 | 182 | 183 | 184 | 185 | 186 | 187 | 188 | 189 | 190 |
| R | F | T | Y | H | M | V | S | G | M |
| 191 | 192 | 193 | 194 | 195 | 196 | 197 | 198 | 199 | 200 |
| S | L | R | P | R | V | N | Y | L | Q |
| 201 | 202 | 203 | 204 | 205 | 206 | 207 | 208 | 209 | 210 |

wherein at least one amino acid at a position in the range of from positions 170 to 210 has been replaced by a cysteine.

In a preferred embodiment the HERV envelope protein comprises a cysteine at position 190. In a more preferred embodiment the HERV envelope protein comprises the amino acid mutation S190C.

In another embodiment the HERV envelope protein alternatively or additionally comprises the sequence VQNWLVEVPTVSPICRFTYHMVSGMSLRP. In another embodiment the HERV envelope protein alternatively or additionally comprises an amino acid sequence having at least 90% sequence identity with SEQ ID NO: 2, wherein the HERV envelope protein comprises a cysteine at a position within said HERV envelope protein that corresponds to amino acid 190 of SEQ ID NO: 2.

In another embodiment the HERV envelope protein comprises or consists of a sequence selected from the group consisting of SEQ ID NO:s 21-35. In a preferred embodiment the HERV envelope protein comprises or consists of a sequence selected from the group consisting of SEQ ID NO:s 22-27, 29-32 and 35.

In a preferred embodiment the composition of the invention comprises an mRNA which has at least 90% sequence identity, and most preferably 100% sequence identity, with any of the sequences according to any one of SEQ ID NO:s 45, 36 or 37. In an even more preferred embodiment the composition of the invention comprises an mRNA which has 100% sequence identity with any of the sequences according to any one of SEQ ID NO:s 45, 36 or 37.

In one embodiment the cell surface expression of the HERV envelope protein (as defined in the previous sections) is increased compared to the cell surface expression of a HERV envelope protein having an amino acid sequence according to SEQ ID: 2.

A further aspect of the invention relates to an mRNA which has at least 90% sequence identity with SEQ ID NO: 45, wherein the mRNA encodes a polypeptide which comprises a HERV-K Gag and a HERV-K envelope (Env) protein; and wherein said HERV Env protein comprises a mutated immune-suppressive domain (ISD) that reduces its immune-suppressive property compared to the wildtype Env ISD (preferred ISD mutations and mutated ISD sequences are disclosed herein in the context of the invention—for example one or more of the ISD mutations L522A, A523Q, N524A, Q525A, N527A, L529A, R530A, Q531A, T532A can be used); and wherein the HERV envelope protein further comprises cysteine instead of serine at amino acid position 190 within said HERV envelope protein. In a preferred embodiment, the aforementioned mRNA is in a composition of the invention. Also provided is the use of the aforementioned mRNA to create a virus like particle (VLP).

A further aspect of the invention relates to a polypeptide having at least 90% sequence identity with the polypeptide encoded by SEQ ID NO: 45, wherein the polypeptide comprises a HERV-K Gag and a HERV-K envelope (Env) protein; and

wherein said HERV Env protein comprises a mutated immune-suppressive domain (ISD) that reduces its immune-suppressive property compared to the wildtype Env ISD (preferred ISD mutations and mutated ISD sequences are disclosed herein in the context of the invention—for example one or more of the ISD mutations L522A, A523Q, N524A, Q525A, N527A, L529A, R530A, Q531A, T532A can be used); and wherein the HERV envelope protein comprises a cysteine instead of serine at position 190 within said HERV envelope protein. In a preferred embodiment, the aforementioned polypeptide is comprised in a virus like particle (VLP). Also provided is a mRNA encoding the aforementioned polypeptide. Preferably, said mRNA is comprised in a composition of the invention.

A further aspect of the invention relates to a polypeptide comprising or consisting of a HERV Env protein having at least 95% sequence identity with SEQ ID NO: 2, wherein the Env protein comprises a mutated immune-suppressive domain (ISD) that reduces its immune-suppressive property compared to the wildtype Env ISD (preferred ISD mutations and mutated ISD sequences are disclosed herein in the context of the invention—for example one or more of the ISD mutations L522A, A523Q, N524A, Q525A, N527A, L529A, R530A, Q531A, T532A can be used); and wherein the HERV envelope protein comprises a cysteine instead of serine at position 190 of SEQ ID NO: 2. In a preferred embodiment, the aforementioned polypeptide is comprised in a virus like particle (VLP). Also provided is a mRNA encoding the aforementioned polypeptide. Preferably, said mRNA is comprised in a composition of the invention.

A further aspect of the invention relates to a polypeptide comprising or consisting of a HERV Env protein, wherein the Env protein comprises a cysteine at position 190 so that the Env protein comprises the amino acid sequence VQNWLVEVPTVSPICRFTYHMVSGMSLRP; and wherein the Env protein further comprises a mutated immune-suppressive domain (ISD) that reduces its immune-suppressive property compared to the wildtype Env ISD (preferred ISD mutations and mutated ISD sequences are disclosed herein in the context of the invention—for example one or more of the ISD mutations L522A, A523Q, N524A, Q525A, N527A, L529A, R530A, Q531A, T532A can be used). In a preferred embodiment, the aforementioned polypeptide is comprised in a virus like particle (VLP). Also provided is a mRNA encoding the aforementioned polypeptide. Preferably, said mRNA is comprised in a composition of the invention.

### Immune-Suppressive Domain (ISD)

An immune-suppressive domain (ISD) can be seen as mechanism used by tumors to balance anti-tumor immune responses, while simultaneously retaining a tumor-promoting inflammatory milieu induced by HERV activation, similar to natural viral infections. The ISD is affecting both the innate and adaptive immune system, due to inhibition of macrophages, NK cells and T cells alike. Although, the detailed mechanism of immune suppression by the ISD is not completely understood yet, a certain capability of this domain to induce IL-10 secretion from peripheral blood mononuclear cells upon contact, and an ability of mutated ISD domains to decrease NF-κB induced gene expression, which was surprisingly found in transfection assays, has been observed as being related to the immune-suppressive and/or inflammatory milieu promoting function of the ISD. In particular, a reduced NF-κB expression due to a mutated ISD domain may be secondary to an immunogenic cell death pathway, leading to an apparent reduction in NF-κB, yet reflecting enhanced immune stimulation.

In one embodiment a mutated ISD of the invention, compared to a wild-type ISD that is not mutated and has an amino acid sequence according to SEQ ID NO: 3 (see Table 1), inhibits the proliferation of human immune cells less, and/or has a reduced or no capability to induce IL-10 secretion from peripheral blood mononuclear cells when contacting said cells with said mutated ISD and/or decreases NF-κB expression.

For an ISD to be inactivated the immune suppressing ability is preferably reduced by 30% or more compared to the immune suppression achieved by the wild-type ISD. Preferably, the ISD is inactivated even by 35% or 40% or more, such as 45% or more, such as 47%, 48% or 49% or more, such as 50% compared to the immune suppression performed by the original, i.e. not mutated ISD. Quantification of the level of immune suppression and/or induction of an inflammatory cell environment may be performed by quantifying levels of IL-10, NF-κB or NF-κB induced genes or other molecules secreted by peripheral blood mononuclear cells. Quantification methods are known in the art and include for example ELISA or FACS based methods.

NF-κB activation/inhibition can specifically be determined by transfecting HEK293T cells with a mixture of a reporter plasmid expressing luciferase upon NF-κB expression, a selected HERV-K Env protein encoding DNA plasmid and lipofectamine. The day after the transfection, cells can be analysed for luciferase expression by addition of luciferase substrate followed by quantifying luminescence. In such an assay, HERV-K with a mutated ISD sequence of SEQ ID NO:4 may for example reduce the basal NF-κB level by approximately 20%, 25%, 30%, or even by approximately 40%, or even by up to 50% or even more.

The ISD segment may be inactivated by mutation or deletion of one or more amino acids. In case the inactivation is performed by a mutation one or more of the amino acids are exchanged with a different amino acid, usually selected among the other 19 naturally occurring amino acids. It is most suitable to replace one or two single amino acids at any one of positions 1, 2, 3, 4, 5, 6, 7, 8, 9, 10, 11, 12, 13 and 14 of ISD SEQ ID NO: 3. A person skilled in the art will have adequate knowledge and experience of which amino acids to exchange to lead them to a satisfactory immune response, optionally through evaluation of initial trials.

Thus, in one embodiment of the present invention the mutated ISD comprises or consists of the amino acid sequence:

| L | A | N | Q | I | N | D | L | R | Q | T | V | I | W |
|---|---|---|---|---|---|---|---|---|---|---|---|---|---|
| 1 | 2 | 3 | 4 | 5 | 6 | 7 | 8 | 9 | 10 | 11 | 12 | 13 | 14 |

wherein one or two single amino acids at any of the positions 1, 2, 3, 4, 5, 6, 7, 8, 9, 10, 11, 12, 13 and 14 are replaced with a different amino acid and preferably by alanine in each instance, to render the ISD inactive. In a preferred embodiment the one or two single amino acids different from the original are selected among naturally occurring amino acids.

Herein it was surprisingly found that when cells were transfected with nucleic acid molecules (e.g. DNA) encoding HERV Env wherein the ISD according to SEQ ID NO: 3 (LANQINDLRQTVIW) comprises mutations of either one of the positions 1, 2, 3, 4, 5, 6, 7, 8, 9, 10, 11, 12, 13 or 14, the number of transfected cells expressing the encoded HERV Env protein with mutated ISD was increased compared to the expression in cells wherein HERV Env with wildtype ISD was transfected (see FIG. 4). Thus, an increased expression of encoded antigenic protein, such as HERV Env, as well as a subsequently increased immune response to the antigen may be achieved when the antigenic protein, such as HERV Env, is encoded with a mutated ISD comprising mutations of either one of the positions 1, 2, 3, 4, 5, 6, 7, 8, 9, 10, 11, 12, 13 or 14, such as particularly of the experimentally tested mutations L522A, A523Q, N524A, Q525A, I526A, N527A, D528A, L529A, R530A, Q531A, T532A, V533A, I534A and W535A

Thus, in another embodiment, the ISD mutation is selected from the group consisting of L522A, A523Q, N524A, Q525A, I526A, N527A, D528A, L529A, R530A, Q531A, T532A, V533A, I534A and W535A. In a preferred embodiment the mutated ISD comprises a sequence according to any one of SEQ ID NO:s 4 or 8-20. In an even more preferred embodiment the ISD mutation is selected from L522A, A523Q, N524A, Q525A, N527A, L529A, R530A, Q531A and T532A. In an even more preferred embodiment the mutated ISD comprises a sequence according to any one of SEQ ID NO:s 4, 8-12, 14-17 or 20.

It may be advantageous to combine the ISD mutations described herein with stabilizing cysteine substitutions as described further above. Thus, in a preferred embodiment the HERV envelope protein comprises an even number of cysteines and alternatively or additionally comprises at least 18 cysteines, and the HERV envelope protein comprises an ISD mutation selected from the group consisting of L522A, A523Q, N524A, Q525A, I526A, N527A, D528A, L529A, R530A, Q531A, T532A, V533A, I534A and W535A, preferably an ISD mutation is selected from the group consisting of L522A, A523Q, N524A, Q525A, N527A, L529A, R530A, Q531A and T532A.

In another preferred embodiment, the HERV envelope protein comprises the amino acid sequence

| G | C | L | M | P | A | V | Q | N | W | L |
|---|---|---|---|---|---|---|---|---|---|---|
| 170 | 171 | 172 | 173 | 174 | 175 | 176 | 177 | 178 | 179 | 180 |

| V | E | V | P | T | V | S | P | I | S |
|---|---|---|---|---|---|---|---|---|---|
| 181 | 182 | 183 | 184 | 185 | 186 | 187 | 188 | 189 | 190 |
| R | F | T | Y | H | M | V | S | G | M |
| 191 | 192 | 193 | 194 | 195 | 196 | 197 | 198 | 199 | 200 |
| S | L | R | P | R | V | N | Y | L | Q |
| 201 | 202 | 203 | 204 | 205 | 206 | 207 | 208 | 209 | 210 |

wherein at least one amino acid at a position in the range of from positions 170 to 210 has been replaced by a cysteine, and the HERV envelope protein comprises an ISD mutation selected from the group consisting of L522A, A523Q, N524A, Q525A, I526A, N527A, D528A, L529A, R530A, Q531A, T532A, V533A, I534A and W535A, preferably an ISD mutation is selected from the group consisting of L522A, A523Q, N524A, Q525A, N527A, L529A, R530A, Q531A and T532A.

In an even more preferred embodiment, the HERV envelope protein comprises a cysteine at position 190, and most preferably the amino acid mutation S190C, and the HERV envelope protein comprises an ISD mutation selected from the group consisting of L522A, A523Q, N524A, Q525A, I526A, N527A, D528A, L529A, R530A, Q531A, T532A, V533A, I534A and W535A, preferably an ISD mutation is selected from the group consisting of L522A, A523Q, N524A, Q525A, N527A, L529A, R530A, Q531A and T532A. In one embodiment the HERV envelope protein comprises the amino acid mutation S190C and the ISD mutation Q525A.

Amino acids are those L-amino acids commonly found in naturally occurring proteins. Amino acid residues are indicated in the present disclosure according to the standard three-letter or one-letter amino acid code. Any amino acid sequence that contains post-translationally modified amino acids may be described as the amino acid sequence that is initially translated and modifications e.g., hydroxylations or glycosylations, shall not be shown explicitly in the amino acid sequence. Any peptide or protein that can be expressed as a sequence modified linkages, cross links and end caps, non-peptidyl bonds, etc., is embraced herein, all as known in the art.

Whereas the person skilled in the art will be able to modify the amino acid sequence by performing any number or form of mutations or deletions, it is currently preferred to exchange a single amino acid of the ISD sequence. This modification involves the replacement of glutamine at position 4 of the ISD with alanine. This change triggers the inactivation of the domain in order to prevent the vaccine itself from producing immunosuppressive effects. Therefore, in one embodiment, the mutated ISD comprised by the RNA of the invention preferably comprises or consists of sequence LANAINDLRQTVIW (SEQ ID NO: 4).

A particularly preferred HERV-K Env sequence containing the ISD disabling mutation is shown in SEQ ID NO: 2 (See also Table 1):

MNPSEMQRKAPPRRRRHRNRAPLTHKMNKMVTSEEQMKLPSTKKA
EPPTWAQLKKLTQLATKYLENTKVTQTPESMLLAALMIVSMVVSL
PMPAGAAAANYTYWAYVPFPPLIRAVTWMDNPIEVYVNDSVWVPG
PIDDRCPAKPEEEGMMINISIGYRYPPICLGRAPGCLMPAVQNWL
VEVPTVSPISRFTYHMVSGMSLRPRVNYLQDFSYQRSLKFRPKGK
PCPKEIPKESKNTEVLVWEECVANSAVILQNNEFGTIIDWAPRGQ
FYHNCSGQTQSCPSAQVSPAVDSDLTESLDKHKHKKLQSFYPWEW
GEKGISTPRPKIVSPVSGPEHPELWRLTVASHHIRIWSGNQTLET
RDRKPFYTVDLNSSLTVPLQSCVKPPYMLVVGNIVIKPDSQTITC
ENCRLLTCIDSTFNWQHRILLVRAREGVWIPVSMDRPWEASPSVH
ILTEVLKGVLNRSKRFIFTLIAVIMGLIAVTATAAVAGVALHSSV
QSVNFVNDWQKNSTRLWNSQSSIDQKLANQINDLRQTVIWMGDRL
MSLEHRFQLQCDWNTSDFCITPQIYNESEHHWDMVRRHLQGREDN
LTLDISKLKEQIFEASKAHLNLVPGTEAIAGVADGLANLNPVTWV
KTIGSTTIINLILILVCLFCLLLVCRCTQQLRRDSDHRERAMMTM
AVLSKRKGGNVGKSKRDQIVTVSV.

It may be preferred to exchange one or more amino acids in a region upstream or downstream of the ISD segment. The mutation is a compensatory mutation intended to preserve the structure of the domain so that it can still work for an infectious virus. Thus, at least one of the amino acids in a region of amino acids upstream or downstream of the ISD may be exchanged with a different amino acid. In case further compensatory mutations should be required a person skilled in the art is aware of effects of such mutations and is able to select the accordingly.

HERV ISD inactivation may be detected and quantified for confirmation via detection of the level of immune response in a subject, which is evoked by administering the composition if the invention. For instance, HERV specific antibodies may be quantified in blood samples from the subject. Conversely, such specific antibodies can be used to label HERV itself to analyze its presentation on cell surfaces in both in vitro and in vivo obtained samples, for instance using FACS. Furthermore, the propagation of immune cells, such as NK and T cells, as well as both extracellular and intracellular stained markers of these activated immune cells in samples from immunized subjects, such as mice may also be analyzed via FACS or other suitable methods. Suitable markers of immune cell activation will be clear to a person skilled in the art. For instance, activated T cells could be detectable via IFNγ, TNFα and CD44 and NK cells could be detectable via CD56.

Herein an immunization effect using the mRNA of the invention and a composition of the invention was confirmed by immunizing mice with mRNA encoding the HERV-K proteins GAG and ENV with a mutated ISD (c.f. immunization schedule in FIG. 5) treated in a prime and boost regimen (day 0 and day 7) and subsequently performing tetramer staining to detect and quantify CD8+ T-cells specific for the HERV-K antigens in mice spleen tissue as well as by ELISA for detection of specific antibodies in mice blood samples (see FIG. 6 or 7, respectively).

The results in FIG. 6 confirm in vivo that immunization with mRNA encoding HERV-K GAG and HERV-K ENV with a mutated ENV ISD (e.g. mutation Q525A) induced CD8+ T-cell responses against both ENV, demonstrated by cells binding Tet18, and against GAG, demonstrated by cells binding Tet90. Further, the results in FIG. 7 confirm that upon immunization of mice with mRNA encoding HERV-K GAG and ENV with an ISDmut (e.g. mutation Q525A) antibody immune responses against both antigen subunits SU and TM in the immunized mice are induced, i.e. against both the HERV-K ENV surface subunit (SU) as well as the HERV-K ENV transmembrane subunit (TM). Therefore, an mRNA encoding an HERV Env protein comprising a mutated ISD may be effectively administered in vivo to generate a robust immune response against the HERV Env antigen and is useful for the vaccination against human endogenous retroviruses, and particularly against HERV-K.

Another exemplary method to test the effectivity of immunizing and/or evoking an immune response by the composition of the invention comprising an mRNA coding for HERV Env with an inactive ISD is assessing the resistance to tumor formation. For instance, a tumor challenge and tumor rejection assay may be performed, wherein subjects, i.e. animals, are injected with tumor cells, for instance cells of tumor cell lines B16F10-GP or CT26 or 4T1 or murine renal carcinoma cells engineered to express parts of the human ERV-K genome, and subsequently treated, i.e. therapeutically vaccinated, with the composition of the invention. After a certain time period, for instance of 1 to 6 weeks, the subjects are analyzed with regard to tumor and/or metastasis formation as well as tumor size and tumor characteristics, e.g. by HERV Env specific staining of dissected tumors. Thus, it can be determined, whether tumor formation was reduced or rejected through the treatment with the composition of the invention and whether tumors expressed HERV Env.

Furthermore, as outlined elsewhere herein, there are at least two ways of antigenic display or antigen provision after cellular contact and uptake of the composition of the invention to initiate an immune response: as MHC class I antigens on the contacted cell's surface, presented to CD8+ T cells, or as MHC class II antigens on professional antigen-presenting cells. Both mechanisms emphasize the importance of surface display for antigen detection in the immunization process.

In this context, the present inventors surprisingly observed that encoded HERV-K Env having an ISD with the point mutation of SEQ ID NO: 4 showed an improved HERV-K cell surface display on HEK293 cells vitro compared to HERV-K Env without the ISD mutation (see FIG. 1). Without wishing to be bound by theory it is expected that the point mutation that renders the ISD inactive, is also the cause for the improved surface display.

Besides the human endogenous retrovirus (HERV) envelope protein or an immunogenic part thereof, the mRNA of the present invention may also encode further proteins. Co-encoded moieties may then for instance experience a kind of pull-along effect towards the cell surface, being displayed along with HERV Env.

Thus, in one embodiment, the present invention may also relate to a composition comprising a transfection agent and an mRNA which encodes at least a human endogenous retrovirus (HERV) envelope protein or an immunogenic part thereof, wherein said HERV envelope protein comprises an immune-suppressive domain (ISD), wherein the HERV Env protein comprises a mutated immune-suppressive domain (ISD) that reduces its immunosuppressive property compared to the wildtype ISD, and wherein said HERV protein is co-expressed with at least one further protein encoded by the same mRNA. The at least one further protein may be conjugated, i.e. fused, to the HERV envelope protein of the invention, which may enable the further protein to be secreted or displayed at the cell surface along with the HERV envelope protein. The at least one further protein may also be expressed separately, i.e. not be fused to the HERV envelope protein of the invention.

The average skilled person is aware of how to obtain fused and not fused co-expressed proteins which are encoded on the same mRNA strand. In one embodiment, the further protein may be directly linked to the HERV envelope protein, or linked to it via a linker also encoded by the mRNA. Suitable linkers are known in the art. In one embodiment, the at least one further protein may be conjugated with the HERV envelope protein of the invention via a linker, wherein said linker is for instance a suitable amino acid sequence, in particular of preferably between 1 and 30, such as between 1 and 10 amino acid residues. Preferred examples of such amino acid sequences include but are not limited to gly-ser linkers.

In some cases, it may be desirable to create a stronger immune response in a subject receiving treatment by the composition of the invention or to evoke immunity against a further antigen in the subject. Thus, in one embodiment, the further encoded protein may be an antigen. In one embodiment, the further encoded protein may be an adjuvant.

In one embodiment, the further encoded protein may be a peptide or protein from a peptide or protein library. The co-display may allow the presentation of the peptide or protein for selection and/or characterization at the cell surface. Advantageously, proteins are more stable when connected to a matrix rather than as free molecules and on the present case, the cell surface acts as a matrix.

By displaying proteins on the cell surface, preparing or purifying the protein also becomes redundant in many instances. Thus, in one embodiment the further encoded protein may be a protein or peptide produced in the host cell, i.e. a eukaryotic cell, wherein the protein or peptide is to be obtained with a certain purity.

Molecules, e.g. peptides or proteins, displayed at the cell surface are freely accessible for substrates or binding partners in activity or binding assays. Thus, in one embodiment, the further encoded protein may be an enzyme or catalytic portion of an enzyme or an antibody or portion thereof.

The displayed molecules are also freely accessible to be bound in cell purification assays or detected in detection assays. Thus, in one embodiment the further encoded protein may be a tag for cell purification, such as an affinity or epitope tag, for instance selected from but not limited to the group consisting of CaM-Tag, CBP-Tag, GST-Tag, MBP-Tag; biotinylating tags such as Avi-Tag, BCCP-Tag, Strep-Tag; His-Tag, FLAG-Tag, Xpress-Tag; T7 epitope Tag and Tap-Tag. In another embodiment the further encoded protein may be a peptide to be detected, such as for instance fluorescent tags, such as for instance GFP, YFP and many others known in the art.

Cancer cells frequently upregulate surface receptors that promote growth and survival. These receptors constitute valid targets for intervention. One strategy involves the delivery of toxic receptor binding agents with the goal of killing those cancer cells with high receptor levels. Thus, to enforce the anticancer effect of a treatment by use of the composition of the invention, the further encoded protein in one embodiment may be an mRNA encoded agent suitable to kill cancer cells, such as for instance a protein toxin.

### Medical Use

As shown in the example, the mRNA of the invention exhibits the surprising beneficial effect of greatly improving cellular surface display of the encoded antigen, i.e. HERV Env. As described in the introductory section, such efficient surface presentation of an antigen is crucial for evoking a response from the immune system by contact with the antigen. Along with the mutation of the ISD rendering it inactive, the increased surface display of the encoded antigen, i.e. HERV Env, is bound to greatly promote an immunologic reactivity of a subject's body against HERV Env. Thus, a subject may be immunized with the composition of the invention that is effective against the development or progression of HERV-related cancer and/or causes the subject's body to fight HERV-related cancer through an immunologic action. The composition of the invention, i.e. the composition comprising a transfection agent and an mRNA which encodes at least a human endogenous retrovirus (HERV) envelope protein or an immunogenic part thereof, with a mutated immune-suppressive domain (ISD) with reduced immune-suppressive property compared to the wildtype ISD, is therefore suitable to be administered in therapy or prophylaxis. The composition may be administered as a treatment or vaccine, or as part of a treatment or vaccine, in order to induce a specific immune response against endogenous retrovirus-related tumor cells and/or immunize a subject against the development or progression of tumors caused by the activity of HERVs.

Thus, in one aspect the invention relates to the composition of the invention for use as a medicament. In another aspect the present invention relates to the composition of the invention for use in the prophylaxis or treatment of a disease, preferably for immunizing a subject against a disease.

In yet another aspect the invention relates to the composition of the invention for the manufacture of a medicament. Yet another aspect of the invention relates to the use of the composition of the invention for the manufacture of a medicament for prophylaxis and/or therapeutic treatment of a disease, preferably for immunizing a subject against a disease.

In yet another aspect the present invention relates to a method of treatment and/or prophylaxis of a disease, preferably of immunizing a subject against a disease, comprising administering to the subject a therapeutically effective amount of the composition of the invention.

There is a wide range of pathologies caused by activity and/or reactivation of ERVs. In one embodiment, the disease, which is subject to prophylaxis and/or therapeutic treatment by the composition of the invention, is an ERV reactivation associated disease or disorder, preferably a HERV reactivation associated disease or disorder. Thus, in a preferred embodiment the disease, which is subject to prophylaxis and/or therapeutic treatment by the composition of the invention, is selected from the group consisting of cancer, HIV and/or associated disorders, rheumatic diseases, neurodegenerative diseases, aging associated diseases, diseases associated with HERV reactivation, chronic inflammation multiple sclerosis, ALS, sarcopenia, kidney diseases and Alzheimer's disease.

For example, HIV can reactivate HERV in human subjects (Jakobsson, Johan, and Michelle Vincendeau. “SnapShot: Human endogenous retroviruses.” Cell 185.2 (2022): 400-400.). Thus, when treating HIV a composition of the present invention this reactivation of HERV in HIV patients is expected to be reduced, consequently providing a health benefit to HIV patients.

In one embodiment, wherein the disease is ALS it may preferably be ALS associated with Transactive response DNA binding protein 43 kDa (TDP-43) and/or its C-terminal fragment, such as ALS associated with increased ubiquitination, hyperphosphorylation, mislocalization, and/or accumulation of TDP-43 and/or its C-terminal fragment. In one embodiment, wherein the disease is Alzheimer's diseases it may preferably be Alzheimer's disease associated with Tau protein expression, Tau protein elevation, Tau protein mislocalization and/or Tau protein aggregation.

The present invention is particularly suitable for use in the prophylaxis and/or treatment of cancer. For instance, the target cancer for HERV-K is prostate cancer, breast cancer, ovarian cancer, lymphomas, melanomas, leukemia and sarcomas; the target cancer for HERV-H is colorectal cancer; the target cancer for HERV-W is testicular cancer, ovarian cancer, breast cancer, lymphomas and leukemia; and the target cancer for HERV-E is lung cancer and liver cancer. Thus, the type of cancer treated or prevented by the present invention is not particularly limited and includes prostate cancer, breast cancer, ovarian cancer, lymphomas, melanomas, leukemia, sarcomas, colorectal cancer, testicular cancer, ovarian cancer, breast cancer, lymphomas, lung cancer, and liver cancer. In a preferred embodiment the cancer is a HERV-expressing cancer. In an even more preferable embodiment said HERV-expressing cancer is selected from the group consisting of a PD-L1-expressing tumor, a cervical cancer, penile cancer, anal cancer, vulvar cancer, vaginal cancer, bladder cancer, breast cancer, clear cell kidney cancer, head/neck squamous cell carcinoma, lung squamous cell carcinoma, melanoma, non-small-cell lung cancer (NSCLC), ovarian cancer, pancreatic cancer, prostate cancer, renal cell cancer, small-cell lung cancer (SCLC), triple negative breast cancer, endometrial cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), and small lymphocytic lymphoma (SLL). Other cancer types may also present suitable targets.

Suitable treatment regimens are suggested in the art and will be clear to a person skilled in the art. For instance, under certain conditions, it may be advantageous to treat a patient using a prime-boost regimen. Thus, the use in the prophylaxis and/or treatment of cancer may for instance comprise the step of priming a subject with the composition of the invention at least 5 days before boosting with the composition of the invention.

The composition of the invention is also suitable for use in the prophylaxis and/or treatment of cancer, which comprises the step of post treating the subject 5 days or more after the exposure of the subject to the composition of the invention, e.g. after vaccination with said composition, with a differently encoded antigen, i.e. the HERV Env protein of the invention, for instance as a VLP derived from an adenovirus, Modified Vaccinia Ankara (MVA) or otherwise virally encoded.

It is preferred that the mRNA of the invention is used as a genetic vaccine, in particular in the prophylaxis and/or treatment of a disease, preferably cancer. Alternatively, the nucleic acid molecule can also be used to produce VLPs, in particular HERV-K VLPs in vitro. The resulting VLPs can then be used in immunotherapy, in particular in the prophylaxis and/or treatment of a disease, preferably cancer. It is understood that also in this context the cancer to be treated is a cancer expressing ERV.

It was found that activation of endogenous retroviruses occurred in organs and tissue of aged individuals and that repression of ERVs alleviated cellular senescence, tissue degeneration and, organismal aging (Liu X, et al.; Resurrection of endogenous retroviruses during aging reinforces senescence. Cell. 2023 Jan. 19; 186(2):287-304.e26). Thus, reactivation of endogenous retroviruses is a hallmark and driving force of cellular senescence and tissue aging. Therefore, in yet another aspect the present invention relates to the composition according to the invention for use in the prevention or slowing of aging and/or of cellular senescence. In yet another aspect the present invention relates to the composition according to the invention for use in the manufacture of a medicament for the prevention or slowing of aging and/or of cellular senescence.

In addition, the present invention also relates to a VLP encoded by the nucleic acid molecule encoding a Gag protein and an HERV envelope protein (Env) or an immunogenic part thereof wherein the native genomic structure connecting Gag and the Env has been replaced by an operative linker. Preferably, said operative linker is p2A. It is further preferred that the HERV is HERV-K. More preferably the HERV is HERV-K with an amino acid sequence as published by Lee et al. (Lee Y N, Bieniasz P D (2007) Reconstitution of an Infectious Human Endogenous Retrovirus. PLoS Pathog 3(1): e10). As mentioned above, the use of such VLPs in immunotherapy is envisaged. Moreover, the invention relates to the nucleic acid molecule or the VLP for use in the prophylaxis and/or treatment of a disease. It is preferred that the disease is cancer. It is understood that the cancer is a cancer expressing the corresponding HERV.

### Pharmaceutical Composition

As outlined above, the composition of the invention is for instance useful in the treatment or prophylaxis of diseases. Thus, in one embodiment, the invention relates to a pharmaceutical composition comprising the composition of the invention.

In some cases, further agents, such as excipients, may be beneficial to improve therapeutic or prophylactic effectivity and/or patients' tolerance when administering the composition of the invention in therapy or prophylaxis as outlined above. Thus, in another aspect the invention relates to a pharmaceutical composition comprising the composition of the invention and comprising a pharmaceutically acceptable excipient.

In some embodiments, pharmaceutically acceptable excipients may be selected as defined elsewhere herein. In some preferred embodiments the pharmaceutical composition of the invention may comprise at least one pharmaceutically acceptable excipient selected from the group consisting of water, sodium chloride, potassium chloride, sucrose, sodium acetate or saline.

### BRIEF DESCRIPTION OF THE FIGURES

FIG. 1: HEK293 cells were transfected with either DNA encoding HERV-K GAG-ENV or HERV-K GAG-ENV ISDmut formulated in JetPEI, or RNA 1 (5′UTR) HBA1-HERV-K Gag Env ISDmut-(3′UTR) HBB-PolyA (70)) (SEQ ID NO: 45) formulated in Lipofectamine Messenger MAX. The same results are expected when transfecting HEK293 cells with RNA having a sequence according to SEQ ID NO: 5. Control samples were left untreated (Control NT) or treated with JetPEI (Control JetPEI) or Lipofectamine Messenger MAX (Control LipoMAX). 17 h post transfection, the cells were stained for HERV-K ENV expression. The graph shows % of HERV-K ENV positive cells of live cells.

FIG. 2: NF-κB activation or inhibition was determined by transfecting HEK293T cells with a mixture of a reporter plasmid expressing luciferase upon NF-κB expression, a DNA plasmid encoding either a HERV-K Env protein with intact ISD (WT) or a HERV-K Env protein with a mutated ISD (ISDmut) and lipofectamine. The day after the transfection, the cells were analyzed for luciferase expression by quantification of luminescence (RLU, relative light units) as a read out for NF-κB activation or inhibition. The displayed data was normalized to baseline activity.

FIG. 3: FIG. 3 is supplementary data to the results shown in FIG. 1. HEK293 cells were transfected with either “RNA 4” encoding 5′UTR of HBA1-HERV-K Gag Env ISDmut (with ISD mutation Q525A)-3′UTR of AES mtRNR1-PolyA (30-linker-70) with an N1-methylpseudouridine (m1Ψ) modification (SEQ ID NO: 36), or “RNA8”, encoding 5′UTR of HBA1-HERV-K Gag Env (with a wild-type ISD without mutation)-3′UTR of AES mtRNR1-PolyA (30-linker-70) with N1-methylpseudouridine (m1Ψ) modification (SEQ ID NO: 37), each formulated in Lipofectamine Messenger MAX. In control samples cells were left untreated (Control NT) or were treated with Lipofectamine Messenger MAX (Control LipoMAX). 24 h post transfection, the cells were stained for HERV-K ENV expression. The graph shows % of HERV K ENV positive cells of live cells.

FIG. 4: HEK293 cells were transfected with DNA encoding HERV-K GAG-ENV (with a wild-type ISD without mutation, sample labelled “WT”) or with DNA encoding HERV-K GAG ENV ISDmut (with an ISD with certain mutations), each formulated in JetPEI. The DNA constructs used were the same as for experiments for data shown in FIG. 1 (c.f. same protein encoding sequence as in SEQ ID NO: 45), except for differing ISD sequences, i.e. different ISD mutations/positions were used. ISD mutations tested for the ISDmut constructs were L522A, A523Q, N524A, Q525A, I526A, N527A, D528A, L529A, R530A, Q531A, T532A, V533A, I534A and W535A (samples labelled with the according mutations; see also ISD sequences in Table 3, SEQ ID NO:s 4 and 8-20). Additionally, constructs with a stabilizing mutation S190C in combination with a wild-type ISD or in combination with a mutated ISD comprising ISD mutation Q525A were tested (samples labelled “WT+S190C” and “Q525A+S190C”). In control samples cells were left untreated (sample labelled “Control NT”) or cells were treated with JetPEI (sample labelled “Control JetPEI”). 24 h post transfection, the cells were stained for HERV-K ENV expression. The graph shows % of HERV-K ENV positive cells of live cells. While these data were generated with DNA constructs encoding the HERV-K proteins, analogous results are expected for RNA constructs encoding these proteins, particularly as protein expression involves the step of transcribing DNA into RNA.

FIG. 5: Schematic representation of the vaccine regimen, wherein CB6F1 WT (wild-type) mice were immunized on day 0 (Prime) with mRNA encoding the HERV-K proteins GAG and ENV ISDmut with N1-methylpseudouridine (m1Ψ) modification (construct according to SEQ ID NO: 36), which can assemble into the form of a VLP with the ISD of the ENV protein mutated (mutation Q525A), represented by (SEQ ID NO: 36). On day 7, the mice received a second dose of mRNA encoding the same antigenic cassette (Boost). Mice were euthanized on day 21 for analysis of immune responses by tetramer staining to detect and quantify T-cells specific for the HERV-K antigens in spleen tissue samples or by ELISA for detection of specific antibodies in mice blood samples. Results are shown in FIGS. 6 and 7.

FIG. 6: The immunization schedule is illustrated in FIG. 5, N=5 mice per group. The data illustrates the frequency (%) of tetramer (c.f. method described in Altman J D, Moss P A, Goulder P J, Barouch D H, McHeyzer-Williams M G, Bell J I, McMichael A J, Davis M M, Phenotypic analysis of antigen-specific T lymphocytes. Science. 1996 Oct. 4; 274(5284):94-6) binding CD8+ T-cells in spleen tissues of immunized mice on day 21 after prime immunization. The frequency of each tetramer (Tet) is shown separately. For tetramer staining on splenocytes, tetramers consist of four coupled MHC class I molecules, loaded with selected antigen specific peptides (from HERV-K) and conjugated to fluorophores. The tetramers bind only to T cells that are specific for the loaded target antigen peptide. As the vaccine used for immunization herein induces activation and expansion of HERV-K antigen-specific T cells, tetramer staining detects the fraction of antigen-specific T cells expanded upon vaccine administration to mice. Tetramer Tet18 binds to CD8+ T-cells that are specific for a HERV-K ENV epitope, and tetramers Tet29 and Tet90 bind to CD8+ T-cells that are specific for HERV-K GAG epitopes. The immunization with mRNA encoding HERV-K GAG and HERV-K ENV with a mutated ENV ISD (mutation Q525A) induced CD8+ T-cell responses against both ENV (i.e. cells bound by Tet18) and GAG (i.e. cells bound by Tet90).

FIG. 7: The immunization schedule is illustrated in FIG. 5, N=5 mice per group. The data illustrates the OD value as determined by ELISA performed with serum samples harvested from immunized mice at day 21 after prime immunization and the signal represents the quantity of antibodies in the mouse serum samples, directed against respective protein subunits. In FIG. 7 A, OD values were determined in a 1:25 dilution of serum samples harvested on day 21 after prime immunization, wherein the OD represents the level of antibodies directed against subunit TM (HERV-K ENV transmembrane subunit). In FIG. 7 B, OD values were determined in a 1:25 dilution of serum samples harvested at day 21 after prime immunization, wherein the OD represents the level of antibodies directed against subunit SU (HERV-K ENV surface subunit). Immunization with mRNA encoding HERV-K GAG and ENV with an ISDmut (mutation Q525A) induced antibody immune responses against both antigen subunits SU and TM in the immunized mice.

### EXAMPLES

### Example 1

### 1.1 Cell Culture

HEK293 cell line originating from a human embryonal kidney culture was generated by transformation with sheared adenovirus type 5 (Ad5) DNA (available from LGC standards (ATCC)). Advantages of this cell line include easy growth and efficient transfection. HEK293 cells were maintained in Dulbecco's Modified Eagle's Medium (DMEM) Glutamax supplemented with 10% heat-inactivated fetal bovine serum (FBS), and 1% penicillin+streptavidin (Pen/Strep). The cell line was maintained at 37° C. with 5% C02 in a humidified atmosphere.

### 1.2 DNA Vectors

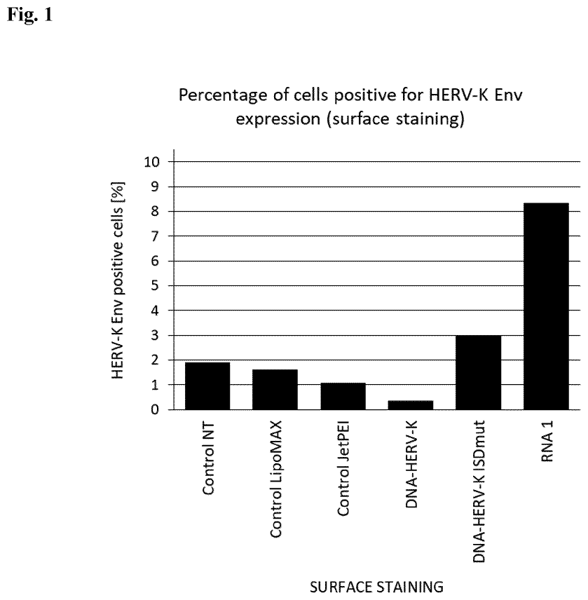

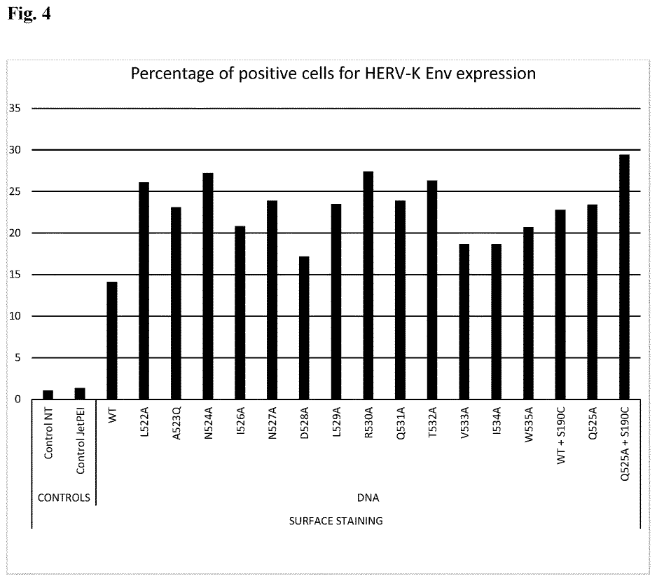

The DNA vector pO6A19 encoding codon optimized HERV-K GAG-ENV (SEQ ID NO: 7), or HERV-K GAG-ENV ISDmut (Q525A) (SEQ ID NO: 6), or HERV-K GAG-ENV ISDmut variants L522A (SEQ ID NO: 8), A523Q (SEQ ID NO: 9), N524A (SEQ ID NO: 10), I526A (SEQ ID NO: 11), N527A (SEQ ID NO: 12), D528A (SEQ ID NO: 13), L529A (SEQ ID NO: 14), R530A (SEQ ID NO: 15), Q531A (SEQ ID NO: 16), T532A (SEQ ID NO: 17), V533A (SEQ ID NO: 18), 1534A (SEQ ID NO: 19), W535A (SEQ ID NO: 20), or HERV-K GAG-ENV ISDWT with S190C (SEQ ID NO: 21), or HERV-K GAG-ENV ISDmut (Q525A) with S190C (SEQ ID NO: 25), was synthesized by GenScript. Targeted genes were preceded by a strong cytomegalovirus promoter and tetracycline operator (TetO) sites and were followed by the SV40 polyadenylation signal. Inserts with genes of interest of the vector encoded for consensus HERV-K GAG gene linked to either Envelope (ENV) or Envelope ISDmut (ENV ISDmut) (Q525A), or ENV ISD variants, or ENV with S190C, or ENV ISDmut (Q525A) with S190C gene via a Glycine/Serine/Glycine (GSG) linker followed by a self-cleaving porcine teschovirus-1 2A peptide (P2A). Synthesized sequence constructs are as outlined in the figure description of FIGS. 1 and 4.

### 1.3 Amplification of DNA

The DNA vectors were transformed by heat-shock into PIR-1 competent E. coli and amplified overnight in LB medium containing 25 ug/mL kanamycin. (Pir gene encodes a replication protein π, which is required to replicate and maintain the DNA vectors containing an R6Kγ origin. The PIR-1 competent E. coli strain contains a mutant allele of the pir gene that maintains a donor vector construct at ~250 copies per cell.) The plasmids were then purified using Nucleobond Xtra Midi kit following the manufacturer's protocol (AH diagnostics).

### 1.4 Generation of RNA

The consensus HERV-K GAG gene was linked to Envelope ISDmut (Env ISDmut) gene via a Glycine/Serine/Glycine (GSG) linker followed by a self-cleaving porcine teschovirus-1 2A peptide (P2A). The synthesized GOI was used as a template by Vectorbuilder and synthesized with flanking 5′UTR, a Kozak sequence, a 3′UTR and a poly-A (SEQ ID NO: 45). The mRNA was manufactured by Vectorbuilder. Synthesized sequence constructs are as outlined in the figure description.

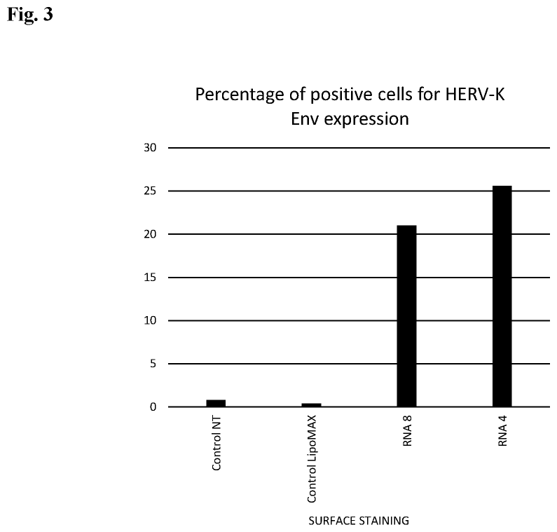

Besides the mRNA construct above, two additional mRNA constructs were synthesized. The consensus HERV-K GAG protein was encoded to be linked to an Envelope (ENV) or Envelope ISDmut (ENV ISDmut) protein via a Glycine/Serine/Glycine (GSG) linker followed by a self-cleaving porcine teschovirus-1 2A peptide (P2A). These synthesized GOIs were used as synthesis templates by Vectorbuilder and synthesized with flanking 5′UTR, a Kozak sequence, a 3′UTR and a poly-A tail as outlined in SEQ ID NO:s 36 and 37. Both of the mRNA constructs were modified with N1-methylpseudouridine (m1Ψ). Synthesized sequence constructs are also outlined in the figure description of FIG. 3.

### 1.5 In Vitro Transfection

HEK293 cells were seeded at 200,000 cells per well in a 24-well plate 23-24 h prior to transfection. Cells were transfected with either 2.4 ug mRNA and 5.6 μL of Lipofectamine Messenger MAX or with DNA at 1 ug and 2 μL JetPEI (Polyplus) per well. Transfected cells were incubated for 17 h (FIG. 1), or 24 h (FIG. 3 and FIG. 4) at 37° C. with 5% CO₂ in a humidified atmosphere.

### 1.6 Surface Staining of Cells

Cells were harvested and washed with PBS with 1% BSA and 0.1% NaN₃. The cells were stained for 30 min with the HERV-K ENV specific antibody HERM 1811-5 (Austral Biologics) conjugated with AlexaFluor647 (Molecular probes). The cells were washed with PBS and incubated with the Viability dye eFluor 780 (eBioscience). The reaction was stopped by adding PBS with 1% BSA and 0.1% NaN₃ and the cells were washed with PBS. The cells were fixed with 2% paraformaldehyde, washed with PBS with 1% BSA and 0.1% NaN₃ and then resuspended in PBS with 1% BSA and 0.1% NaN₃. The samples were run on Fortessa 3 or 5 for flow cytometry and results were analysed using FlowJo software V10.7.1 or V10.8.1. The results are shown in FIGS. 1, 3 and 4.

### Example 2

### LNPs

LNPs can be produced by taking a nucleotide, e.g. mRNA, of the invention and following the methodology disclosed for example in Leung et al., (Leung, et al., 2015, Microfluidic Mixing: A General Method for Encapsulating Macromolecules in Lipid Nanoparticle Systems. The Journal of Physical Chemistry B, 119(28), 8698-8706), Ripoll et al. (Ripoll et al., 2022, Optimal self-assembly of lipid nanoparticles (LNP) in a ring micromixer, Sci Rep 12, 9483) or another suitable method known in the art.

In a preferred embodiment, the LNP are formulated by mixing an mRNA of the invention with a lipid composition comprising at least one cationic lipid. More preferably the mRNA is mixed with a lipid composition comprising DOSPA (2,3-dioleoyloxy-N-[2(sperminecarboxamido)ethyl]-N,N-dimethyl-1-propaniminium trifluoroacetate) and DOPE, wherein preferably DOSPA and DOPE is in a molar ratio of between 2:1 to 4:1. A formulation as outlined above in Examples 1.4 and 1.5 can be used to bring the mRNA into the target cells.

### Example 3

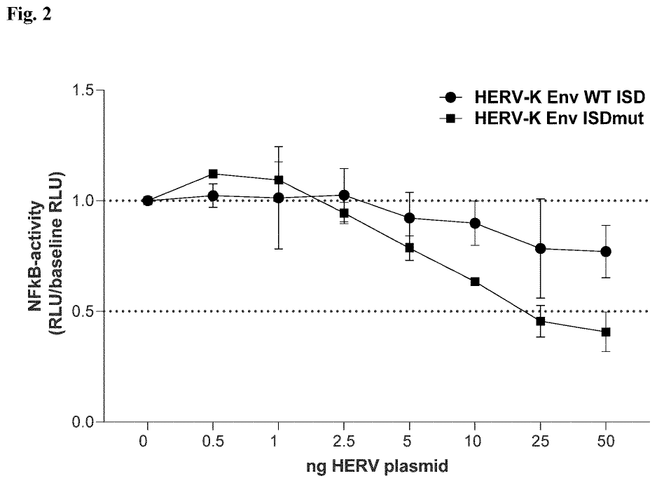

NF-κB activation/inhibition was determined by transfecting HEK293T cells in a 96-well plate with a mixture of 50 ng reporter plasmid expressing luciferase upon NF-κB expression, 0-50 ng of a selected HERV-K Env protein encoding DNA plasmid and 0.6 μl lipofectamine per well. The total DNA amount was adjusted to 100 ng using the empty plasmid pO6A5tetO empty (IPT22). For the results shown in FIG. 2, the cells were transfected with the following HERV-K Env plasmids: HERV-K WT IPT24 plasmid with p06A5-(TetO)-CMV-coHERV-K-P2TS and HERV-K ISDmut IP27 plasmid with p06A5-(TetO)-CMV-ISDmut-coHERV-K-P2TS).

The day after the transfection, cells were analyzed for luciferase expression by addition of SteadyLite luciferase substrate (PerkinElmer) followed by quantifying luminescence. Luminescence can be measured by a suitable method known in the art. The experiment may for instance be carried out essentially as described in the publication by Mendez et al. (2020) (Mendez J M, Keestra-Gounder A M. NF-κB-dependent Luciferase Activation and Quantification of Gene Expression in Salmonella Infected Tissue Culture Cells. J Vis Exp. 2020, Jan. 12) or according to a protocol derived from said protocol. In the assay, HERV-K with the mutated ISD sequence of SEQ ID NO:4 exhibited a reduction of NF-κB as measured in a luciferase assay compared to a cell transfected with a control plasmid by about 50%. The results are shown in FIG. 2.

### Example 4

### 4.1 Animal Procedures and Serum Isolation

All animal procedures were performed in accordance with the national guidelines of Denmark and the experimental procedures were approved by the National Animal Experimental Inspectorate (Dyreforsogstilsynet) of Denmark. Female CB6F1 mice were obtained at 6-8 weeks of age from Envigo and housed at the Panum Institute, University of Copenhagen, for at least one week before conducting any experiments.

### 4.2 Immunization and Serum Isolation

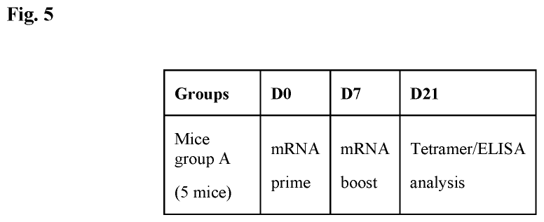

Mice were immunized in a homologous prime-boost regimen with mRNA-HERV-K GAG-ENV ISDmut with N1-Methylpseudouridine (m1Ψ) (SEQ ID NO: 36). A prime immunization was injected on day 0, followed by a boost immunization on day 7. Immune responses were analyzed 14 days after the boost, i.e. on day 21 after prime immunization on day 0. The immunization schedule is shown in FIG. 5.

Before injection, the mRNA was diluted in OptiMEM and mixed with Lipofectamine RNAiMAX at a ratio of 1:2 of μg mRNA to μL RNAiMAX. Each mouse received a dose of 3.5 μg mRNA in 200 μL solution injected intravenously (i.v.). On day 21, blood samples were taken by bleeding from the cheek of the animals. Serum was isolated from the obtained blood samples by two consecutive centrifugation steps, each for 8 min at 800 g and at 8° C. At the end of the immunization studies, mice were euthanized by cervical dislocation.

### 4.3 Splenocyte Suspension

Spleens were removed aseptically from euthanized mice (at the end of the immunization studies) and transferred to RPMI 1640 GlutaMax supplemented with 10% heat-inactivated FBS, 1% Pen/Strep, and 1% Na Pyruvate (complete RPMI). Single-cell suspensions were obtained by pressing the spleens through a fine mesh (mesh size 70 μm), followed by centrifugation of the splenocytes.

### 4.4 Tetramer Staining

Mouse splenocytes were resuspended in PBS with 1% BSA, 0.1% NaN₃ (FACS buffer) and 50 nM Dasatinib and incubated for 30 min at 37° C. and 5% CO₂. The splenocytes were then centrifuged and then incubated with the respective tetramer, wherein 0.08 μg to 0.25 μg of the relevant tetramers in FACS buffer with 50 nM of Dasatinib were added to 500,000 cells at 37° C., 5% C02 for 15 min in the dark.:
TET18: peptide TYHMVSGMSL with H2Kd MHC class I monoclonal antibody, labelled with Brilliant Violet BV421 provided by Immunitrack;
TET29: peptide QNVDYNQL with H2Kb MHC class I monoclonal antibody, labelled with Allophycocyanin fluorophore (APC) provided by Immunitrack; and
TET90: peptide EPYPDFVARL with H2Kb MHC class I monoclonal antibody, labelled with Phycoerythrin fluorophore (PE) provided by Immunitrack.

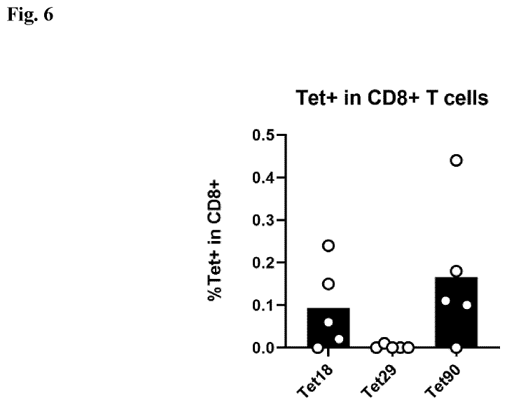

In FACS Buffer with 50 nM of Dasatinib surface antibodies anti-CD8b antibody CD8b-BV510, anti-CD4 antibody CD4-PE-Cy7 and anti-B220/CD45R antibody B220-PerCP-Cy5.5, as well as viability dye (Efluor780) were added on top the tetramer mix and incubated for 20 min at 4° C. in the dark. Cells were then washed and fixed with 1% of paraformaldehyde (PFA) for 15 min at 4° C. in the dark. Fixed samples from tetramer staining were run on a Fortessa 3 flow cytometer and analyzed using FlowJo software V10.7.1. The results are shown in FIG. 6.

### 4.5 ELISA

MaxiSorp (NUNC) flat bottom plates were coated with proteins of the HERV-K ENV (envelope protein) transmembrane subunit (TM) or HERV-K surface subunit (SU) at 2 μg/mL in PBS overnight at 4° C. Plates were washed three times with wash buffer (PBS+354 mM NaCl+0.1% Tween20, pH 7.2) and then blocked for 1 h at room temperature (about 21° C.) using Blocking buffer (PBS+354 mM NaCl+5 g/L BSA+0.05% Tween20, pH 7.2). After removing the blocking buffer, and washing the plates three times (with wash buffer), 100 μL/well serum samples diluted at 1:25 in blocking buffer were added to the wells, followed by 1 h of incubation at room temperature (about 21° C.). Plates were washed three times with wash buffer and horseradish peroxidase (HRP)-conjugated polyclonal anti-mouse/IgG secondary antibody (Dako, P0260) was added at a dilution of 1:2000 in blocking buffer and samples were incubated for 1 h at room temperature (about 21° C.). After three washing steps with wash buffer, 50 μL of 3, 3′, 5, 5′-tetramethylbenzidine (TMB) PLUS 2 chromogenic substrate for horseradish peroxidase (Kem-en-tec, 4395A) was added. The colorimetric reaction was stopped after 6 min by addition of 50 μL of 0.2 M H₂SO₄.

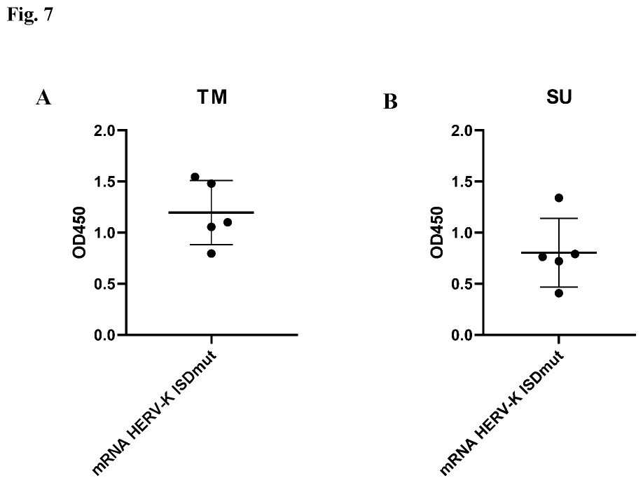

The color intensity in each well was determined by absorbance (OD) quantification at a wavelength of 450 nm (in a SpectraMax Microplate Reader). The background signal measured for samples obtained from non-immunized mice was subtracted for each sample. The results are shown in FIG. 7.

The various aspects and implementations have been described in conjunction with various embodiments herein. However, other variations to the disclosed embodiments can be understood and effected by those skilled in the art in practicing the claimed subject-matter, from a study of the drawings, the disclosure, and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measured cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems.

Sequences are disclosed in the main body of the description and in a separate sequence listing according to WIPO standard ST.25. A SEQ ID specified with a specific number should be the same in the main body of the description and in the separate sequence listing. By way of example SEQ ID no.: 1 should define the same sequence in both, the main body of the description and in the separate sequence listing. Should there be a discrepancy between a sequence definition in the main body of the description and the separate sequence listing (if e.g. SEQ ID no.: 1 in the main body of the description erroneously corresponds to SEQ ID no.: 2 in the separate sequence listing) then a reference to a specific sequence in the application, in particular of specific embodiments, is to be understood as a reference to the sequence in the main body of the application and not to the separate sequence listing.

In other words, a discrepancy between a sequence definition/designation in the main body of the description and the separate sequence listing is to be resolved by correcting the separate sequence listing to the sequences and their designation disclosed in the main body of the application which includes the description, examples, figures and claims.

### Example 5

A composition according to the invention can be produced by following examples 1.4 and 2 as outlined above.

In preferred embodiments this composition or any other composition of the invention described herein can be administered to patients suffering from age related diseases. It is known that retroviruses including HERV can be reactivated in elderly persons and cause disease symptoms (see Zlotorynski, E. Younger endogenous retroviruses make us older. Nat Rev Mol Cell Biol 24, 165 (2023)).

Accordingly, an administering of the compositions of the invention to such patients is expected to ameliorate these symptoms. The skilled artisan can test several dosages to find the amount that is sufficient to ameliorate these symptoms.

TABLE 1
Peptide sequences of HERV-K Env with mutated ISD, wild type ISD and ISD variant

| SEQ | | |
|---|---|---|
| ID | | |
| NO: | Description | Sequence |
| 1 | HERV-K | MNPSEMQRKAPPRRRRHRNRAPLTHKMNKMVTSEEQMKLPSTKKAEPPTWAQLKKLTQLAT |
| | Env WT | KYLENTKVTQTPESMLLAALMIVSMVVSLPMPAGAAAANYTYWAYVPFPPLIRAVTWMDNP |
| | | IEVYVNDSVWVPGPIDDRCPAKPEEEGMMINISIGYRYPPICLGRAPGCLMPAVQNWLVEV |
| | | PTVSPISRFTYHMVSGMSLRPRVNYLQDFSYQRSLKFRPKGKPCPKEIPKESKNTEVLVWE |
| | | ECVANSAVILQNNEFGTIIDWAPRGQFYHNCSGQTQSCPSAQVSPAVDSDLTESLDKHKHK |
| | | KLQSFYPWEWGEKGISTPRPKIVSPVSGPEHPELWRLTVASHHIRIWSGNQTLETRDRKPF |
| | | YTVDLNSSLTVPLQSCVKPPYMLVVGNIVIKPDSQTITCENCRLLTCIDSTENWQHRILLV |
| | | RAREGVWIPVSMDRPWEASPSVHILTEVLKGVLNRSKRFIFTLIAVIMGLIAVTATAAVAG |
| | | VALHSSVQSVNFVNDWQKNSTRLWNSQSSIDQKLANQINDLRQTVIWMGDRLMSLEHRFQL |
| | | QCDWNTSDFCITPQIYNESEHHWDMVRRHLQGREDNLTLDISKLKEQIFEASKAHLNLVPG |
| | | TEAIAGVADGLANLNPVTWVKTIGSTTIINLILILVCLFCLLLVCRCTQQLRRDSDHRERA |
| | | MMTMAVLSKRKGGNVGKSKRDQIVTVSV |
| 2 | HERV-K | MNPSEMQRKAPPRRRRHRNRAPLTHKMNKMVTSEEQMKLPSTKKAEPPTWAQLKKLTQLAT |
| | Env | KYLENTKVTQTPESMLLAALMIVSMVVSLPMPAGAAAANYTYWAYVPFPPLIRAVTWMDNP |
| | ISDmut | IEVYVNDSVWVPGPIDDRCPAKPEEEGMMINISIGYRYPPICLGRAPGCLMPAVQNWLVEV |
| | (ISD | PTVSPISRFTYHMVSGMSLRPRVNYLQDFSYQRSLKFRPKGKPCPKEIPKESKNTEVLVWE |
| | sequence | ECVANSAVILQNNEFGTIIDWAPRGQFYHNCSGQTQSCPSAQVSPAVDSDLTESLDKHKHK |
| | underlined) | KLQSFYPWEWGEKGISTPRPKIVSPVSGPEHPELWRLTVASHHIRIWSGNQTLETRDRKPF |
| | | YTVDLNSSLTVPLQSCVKPPYMLVVGNIVIKPDSQTITCENCRLLTCIDSTENWQHRILLV |
| | | RAREGVWIPVSMDRPWEASPSVHILTEVLKGVLNRSKRFIFTLIAVIMGLIAVTATAAVAG |
| | | VALHSSVQSVNFVNDWQKNSTRLWNSQSSIDQKLANAINDLRQTVIWMGDRLMSLEHRFQL |
| | | QCDWNTSDFCITPQIYNESEHHWDMVRRHLQGREDNLTLDISKLKEQIFEASKAHLNLVPG |
| | | TEAIAGVADGLANLNPVTWVKTIGSTTIINLILILVCLFCLLLVCRCTQQLRRDSDHRERA |
| | | MMTMAVLSKRKGGNVGKSKRDQIVTVSV |
| 3 | WT ISD of | LANQINDLRQTVIW |
| | HERV-K | |
| | Env | |
| 4 | ISDmut of | LANAINDLRQTVIW |
| | HERV-K | |
| | Env, | |
| | mutation | |
| | Q525A | |

TABLE 2
Nucleotide sequences of mRNA coding for HERV-K Env (ISD coding sequences
underlined) and Gag

| SEQ | | |
|---|---|---|
| ID | | |
| NO: | Description | Sequence |
| 5 | Variant- | AGACTCTTCTGGTCCCCACAGACTCAGAGAGAAGCCACCATGGGCCAGACCAAGAGCAAGA |
| | (5′UTR) | TCAAGTCTAAGTACGCCAGCTACCTGAGCTTCATCAAGATCCTGCTGAAGCGCGGAGGCGT |
| | HBA1- | GAAGGTGTCCACCAAGAACCTGATCAAGCTGTTCCAGATCATCGAGCAGTTCTGCCCCTGG |
| | HERV-K | TTTCCTGAGCAGGGCACCCTGGATCTGAAGGACTGGAAGCGGATCGGCAAAGAGCTGAAGC |
| | Gag Env | AGGCCGGCAGAAAGGGCAACATCATCCCTCTGACCGTGTGGAACGACTGGGCCATCATTAA |
| | ISDmut- | GGCCGCTCTGGAACCCTTCCAGACCGAAGAGGATAGCGTGTCCGTGTCTGATGCCCCTGGC |
| | (3′UTR) | AGCTGCATCATCGACTGCAACGAGAACACCCGGAAGAAGTCCCAGAAAGAGACAGAGGGCC |
| | HBB-PolyA | TGCACTGCGAGTATGTGGCCGAACCTGTGATGGCTCAGAGCACCCAGAACGTGGACTACAA |
| | (70) | CCAGCTGCAAGAAGTGATCTACCCCGAGACACTGAAGCTGGAAGGCAAGGGCCCTGAACTC |
| | (sequence | GTGGGCCCTTCTGAGTCTAAGCCCAGAGGCACATCTCCTCTGCCTGCTGGACAGGTGCCAG |
| | encoding | TGACACTGCAGCCCCAGAAACAAGTGAAAGAGAACAAGACCCAGCCTCCTGTGGCCTACCA |
| | ISD in bold) | GTATTGGCCTCCAGCCGAGCTGCAGTACAGACCTCCTCCAGAGAGCCAGTACGGCTACCCT |
| | | GGAATGCCTCCTGCTCCTCAAGGCAGAGCCCCTTATCCTCAGCCTCCTACCAGACGGCTGA |
| | | ACCCTACAGCTCCTCCTAGCAGACAGGGCTCTGAGCTGCACGAGATCATTGACAAGAGCCG |
| | | GAAAGAGGGCGACACCGAGGCTTGGCAGTTTCCCGTTACACTGGAACCCATGCCTCCAGGC |
| | | GAAGGCGCTCAAGAAGGCGAACCTCCTACAGTGGAAGCCCGGTACAAGAGCTTCAGCATCA |
| | | AAATGCTGAAGGACATGAAGGAAGGCGTCAAGCAGTACGGCCCTAACAGCCCCTACATGCG |
| | | GACCCTGCTGGATTCTATTGCCCACGGCCACCGGCTGATCCCTTACGATTGGGAGATCCTG |
| | | GCCAAGTCCTCTCTGAGCCCTAGCCAGTTCCTGCAGTTCAAGACCTGGTGGATCGACGGCG |
| | | TGCAAGAACAAGTGCGGCGGAACAGAGCCGCCAATCCTCCTGTGAACATCGACGCCGATCA |
| | | GCTGCTCGGAATCGGCCAGAATTGGAGCACCATCTCTCAGCAGGCCCTGATGCAGAACGAG |
| | | GCCATTGAACAAGTCCGGGCCATCTGCCTGAGAGCCTGGGAGAAGATTCAGGACCCCGGCA |
| | | GCACATGCCCCAGCTTTAATACCGTTCGGCAGGGCAGCAAAGAGCCCTATCCTGACTTTGT |
| | | GGCCCGGCTGCAGGATGTGGCCCAGAAGTCTATTGCCGACGAGAAGGCCCGGAAAGTGATC |
| | | GTGGAACTGATGGCCTACGAGAACGCCAATCCAGAGTGCCAGAGCGCCATCAAGCCCCTGA |
| | | AGGGAAAAGTGCCTGCCGGCTCCGATGTGATCAGCGAGTACGTGAAGGCCTGCGACGGAAT |
| | | TGGCGGAGCCATGCACAAAGCCATGCTGATGGCACAGGCCATCACTGGCGTTGTGCTCGGA |
| | | GGACAAGTTCGGACCTTTGGCGGCAAGTGCTACAACTGTGGCCAGATCGGACACCTGAAGA |
| | | AAAACTGCCCCGTGCTGAACAAGCAGAACATCACCATCCAGGCCACCACCACCGGCAGAGA |
| | | ACCTCCAGATCTGTGCCCTAGATGCAAGAAGGGCAAGCACTGGGCCAGCCAGTGCAGAAGC |
| | | AAGTTCGACAAGAACGGCCAGCCTCTGAGCGGCAACGAGCAAAGAGGACAGCCTCAGGCTC |
| | | CTCAGCAGACCGGCGCATTTCCAATCCAGCCTTTCGTGCCCCAAGGCTTCCAGGGACAACA |
| | | GCCTCCACTGAGCCAAGTGTTCCAGGGCATTAGCCAGCTGCCTCAGTACAACAACTGCCCT |
| | | CCACCTCAGGCTGCCGTGCAGCAGGGATCAGGCGCTACAAATTTTAGCCTGCTGAAGCAGG |
| | | CAGGGGATGTGGAGGAAAACCCCGGACCTATGAACCCCAGCGAGATGCAGAGAAAGGCCCC |
| | | ACCTAGACGGCGGAGACACAGAAATAGAGCCCCTCTGACACACAAGATGAACAAGATGGTC |
| | | ACCAGCGAGGAACAGATGAAGCTGCCCAGCACCAAGAAGGCCGAGCCTCCAACATGGGCCC |
| | | AGCTGAAGAAACTGACCCAGCTGGCCACCAAGTACCTGGAAAACACCAAAGTGACCCAGAC |
| | | ACCTGAGAGCATGCTGCTGGCCGCTCTGATGATCGTGTCCATGGTGGTGTCCCTGCCTATG |
| | | CCTGCTGGTGCCGCCGCTGCCAATTACACATACTGGGCCTACGTGCCCTTTCCTCCTCTGA |
| | | TTCGGGCCGTGACCTGGATGGACAACCCCATCGAAGTGTACGTGAACGACAGCGTGTGGGT |
| | | GCCCGGACCAATCGACGATAGATGTCCCGCCAAGCCTGAGGAAGAGGGCATGATGATCAAC |
| | | ATCAGCATCGGCTACCGGTATCCTCCAATCTGCCTGGGCAGAGCCCCTGGCTGTCTTATGC |
| | | CAGCTGTGCAGAACTGGCTGGTGGAAGTGCCTACCGTGTCTCCCATCAGCCGGTTCACCTA |
| | | CCACATGGTGTCCGGCATGAGCCTGCGGCCTAGAGTGAACTACCTGCAGGACTTCAGCTAC |
| | | CAGCGGAGCCTGAAGTTCAGACCCAAGGGCAAGCCCTGTCCTAAAGAGATCCCCAAAGAGT |
| | | CCAAGAACACCGAGGTGCTCGTGTGGGAAGAGTGCGTGGCCAATTCTGCCGTGATCCTGCA |
| | | GAACAACGAGTTCGGCACCATCATCGACTGGGCCCCTAGAGGCCAGTTCTACCACAATTGC |
| | | AGCGGCCAGACACAGAGCTGCCCTAGCGCTCAAGTGTCACCAGCCGTGGATAGCGATCTGA |
| | | CCGAGAGCCTGGACAAGCACAAACACAAGAAGCTGCAGAGCTTCTACCCCTGGGAGTGGGG |
| | | CGAGAAGGGCATCTCTACACCCAGACCTAAGATCGTGTCACCCGTGTCCGGACCTGAGCAC |
| | | CCTGAACTTTGGAGACTGACAGTGGCCAGCCACCACATCAGAATTTGGAGCGGCAATCAGA |
| | | CCCTGGAAACCCGGGACAGAAAGCCCTTCTACACCGTGGACCTGAACAGCAGCCTGACCGT |
| | | GCCTCTCCAGAGCTGTGTGAAGCCTCCTTACATGCTGGTCGTGGGCAACATCGTGATCAAG |
| | | CCCGACAGCCAGACCATCACATGCGAGAACTGCAGACTGCTGACCTGCATCGACAGCACCT |
| | | TCAACTGGCAGCACCGGATCCTGCTCGTGCGAGCTAGAGAAGGCGTGTGGATCCCCGTGTC |
| | | TATGGACAGACCTTGGGAAGCTAGCCCCAGCGTGCACATCCTGACAGAGGTGCTGAAGGGC |
| | | GTGCTGAACAGAAGCAAGCGGTTCATCTTCACCCTGATCGCCGTGATCATGGGCCTGATTG |
| | | CTGTGACAGCCACAGCTGCTGTTGCTGGCGTGGCCCTGCATAGCTCTGTGCAGAGCGTGAA |
| | | CTTCGTGAACGATTGGCAGAAGAACAGCACCCGGCTGTGGAACAGCCAGAGCAGCATCGAT |
| | | CAGAAGCTGGCCAACGCCATCAACGACCTGCGGCAGACAGTGATCTGGATGGGCGACAGAC |
| | | TGATGAGCCTGGAACACCGGTTCCAGCTGCAGTGCGACTGGAATACCAGCGACTTCTGCAT |
| | | CACCCCTCAGATCTACAACGAGAGCGAGCACCACTGGGATATGGTCCGAAGGCATCTGCAG |
| | | GGCAGAGAGGACAACCTGACACTGGACATCAGCAAGCTGAAAGAGCAGATCTTCGAGGCCA |
| | | GCAAGGCCCACCTGAATCTGGTGCCTGGAACCGAAGCTATTGCCGGCGTTGCAGATGGCCT |
| | | GGCCAATCTGAATCCTGTGACCTGGGTCAAGACCATCGGCAGCACCACAATCATCAACCTG |
| | | ATCCTGATCCTCGTGTGCCTGTTCTGCCTGCTGCTTGTGTGCAGATGCACCCAGCAGCTGA |
| | | GAAGAGACAGCGACCATAGAGAACGGGCCATGATGACCATGGCCGTGCTGAGCAAGAGAAA |
| | | AGGCGGCAACGTGGGCAAGAGCAAGCGGGATCAGATCGTGACCGTGTCCGTGTGATGAGCT |
| | | CGCTTTCTTGCTGTCCAATTTCTATTAAAGGTTCCTTTGTTCCCTAAGTCCAACTACTAAA |
| | | CTGGGGGATATTATGAAGGGCCTTGAGCATCTGGATTCTGCCTAATAAAAAACATTTATTT |
| | | TCATTGCAAAAAAAAAAAAAAAAAAAAAAAAAAAAAAAAAAAAAAAAAAAAAAAAAAAAAA |
| | | AAAAAAAAAAAAAAAA |
| 45 | (5′UTR) | ACTCTTCTGGTCCCCACAGACTCAGAGAGAAGCCACCATGGGCCAGACCAAGAGCAAGATC |
| | HBA1- | AAGTCTAAGTACGCCAGCTACCTGAGCTTCATCAAGATCCTGCTGAAGCGCGGAGGCGTGA |
| | HERV-K | AGGTGTCCACCAAGAACCTGATCAAGCTGTTCCAGATCATCGAGCAGTTCTGCCCCTGGTT |
| | Gag Env | TCCTGAGCAGGGCACCCTGGATCTGAAGGACTGGAAGCGGATCGGCAAAGAGCTGAAGCAG |
| | ISDmut- | GCCGGCAGAAAGGGCAACATCATCCCTCTGACCGTGTGGAACGACTGGGCCATCATTAAGG |
| | (3′UTR) | CCGCTCTGGAACCCTTCCAGACCGAAGAGGATAGCGTGTCCGTGTCTGATGCCCCTGGCAG |
| | = “RNA 1” | CTGCATCATCGACTGCAACGAGAACACCCGGAAGAAGTCCCAGAAAGAGACAGAGGGCCTG |
| | (sequence | CACTGCGAGTATGTGGCCGAACCTGTGATGGCTCAGAGCACCCAGAACGTGGACTACAACC |
| | encoding | AGCTGCAAGAAGTGATCTACCCCGAGACACTGAAGCTGGAAGGCAAGGGCCCTGAACTCGT |
| | ISD in bold) | GGGCCCTTCTGAGTCTAAGCCCAGAGGCACATCTCCTCTGCCTGCTGGACAGGTGCCAGTG |
| | | ACACTGCAGCCCCAGAAACAAGTGAAAGAGAACAAGACCCAGCCTCCTGTGGCCTACCAGT |
| | | ATTGGCCTCCAGCCGAGCTGCAGTACAGACCTCCTCCAGAGAGCCAGTACGGCTACCCTGG |
| | | AATGCCTCCTGCTCCTCAAGGCAGAGCCCCTTATCCTCAGCCTCCTACCAGACGGCTGAAC |
| | | CCTACAGCTCCTCCTAGCAGACAGGGCTCTGAGCTGCACGAGATCATTGACAAGAGCCGGA |
| | | AAGAGGGCGACACCGAGGCTTGGCAGTTTCCCGTTACACTGGAACCCATGCCTCCAGGCGA |
| | | AGGCGCTCAAGAAGGCGAACCTCCTACAGTGGAAGCCCGGTACAAGAGCTTCAGCATCAAA |
| | | ATGCTGAAGGACATGAAGGAAGGCGTCAAGCAGTACGGCCCTAACAGCCCCTACATGCGGA |
| | | CCCTGCTGGATTCTATTGCCCACGGCCACCGGCTGATCCCTTACGATTGGGAGATCCTGGC |
| | | CAAGTCCTCTCTGAGCCCTAGCCAGTTCCTGCAGTTCAAGACCTGGTGGATCGACGGCGTG |
| | | CAAGAACAAGTGCGGCGGAACAGAGCCGCCAATCCTCCTGTGAACATCGACGCCGATCAGC |
| | | TGCTCGGAATCGGCCAGAATTGGAGCACCATCTCTCAGCAGGCCCTGATGCAGAACGAGGC |
| | | CATTGAACAAGTCCGGGCCATCTGCCTGAGAGCCTGGGAGAAGATTCAGGACCCCGGCAGC |
| | | ACATGCCCCAGCTTTAATACCGTTCGGCAGGGCAGCAAAGAGCCCTATCCTGACTTTGTGG |
| | | CCCGGCTGCAGGATGTGGCCCAGAAGTCTATTGCCGACGAGAAGGCCCGGAAAGTGATCGT |
| | | GGAACTGATGGCCTACGAGAACGCCAATCCAGAGTGCCAGAGCGCCATCAAGCCCCTGAAG |
| | | GGAAAAGTGCCTGCCGGCTCCGATGTGATCAGCGAGTACGTGAAGGCCTGCGACGGAATTG |
| | | GCGGAGCCATGCACAAAGCCATGCTGATGGCACAGGCCATCACTGGCGTTGTGCTCGGAGG |
| | | ACAAGTTCGGACCTTTGGCGGCAAGTGCTACAACTGTGGCCAGATCGGACACCTGAAGAAA |
| | | AACTGCCCCGTGCTGAACAAGCAGAACATCACCATCCAGGCCACCACCACCGGCAGAGAAC |
| | | CTCCAGATCTGTGCCCTAGATGCAAGAAGGGCAAGCACTGGGCCAGCCAGTGCAGAAGCAA |
| | | GTTCGACAAGAACGGCCAGCCTCTGAGCGGCAACGAGCAAAGAGGACAGCCTCAGGCTCCT |
| | | CAGCAGACCGGCGCATTTCCAATCCAGCCTTTCGTGCCCCAAGGCTTCCAGGGACAACAGC |
| | | CTCCACTGAGCCAAGTGTTCCAGGGCATTAGCCAGCTGCCTCAGTACAACAACTGCCCTCC |
| | | ACCTCAGGCTGCCGTGCAGCAGGGATCAGGCGCTACAAATTTTAGCCTGCTGAAGCAGGCA |
| | | GGGGATGTGGAGGAAAACCCCGGACCTATGAACCCCAGCGAGATGCAGAGAAAGGCCCCAC |
| | | CTAGACGGCGGAGACACAGAAATAGAGCCCCTCTGACACACAAGATGAACAAGATGGTCAC |
| | | CAGCGAGGAACAGATGAAGCTGCCCAGCACCAAGAAGGCCGAGCCTCCAACATGGGCCCAG |
| | | CTGAAGAAACTGACCCAGCTGGCCACCAAGTACCTGGAAAACACCAAAGTGACCCAGACAC |
| | | CTGAGAGCATGCTGCTGGCCGCTCTGATGATCGTGTCCATGGTGGTGTCCCTGCCTATGCC |
| | | TGCTGGTGCCGCCGCTGCCAATTACACATACTGGGCCTACGTGCCCTTTCCTCCTCTGATT |
| | | CGGGCCGTGACCTGGATGGACAACCCCATCGAAGTGTACGTGAACGACAGCGTGTGGGTGC |
| | | CCGGACCAATCGACGATAGATGTCCCGCCAAGCCTGAGGAAGAGGGCATGATGATCAACAT |
| | | CAGCATCGGCTACCGGTATCCTCCAATCTGCCTGGGCAGAGCCCCTGGCTGTCTTATGCCA |
| | | GCTGTGCAGAACTGGCTGGTGGAAGTGCCTACCGTGTCTCCCATCAGCCGGTTCACCTACC |
| | | ACATGGTGTCCGGCATGAGCCTGCGGCCTAGAGTGAACTACCTGCAGGACTTCAGCTACCA |
| | | GCGGAGCCTGAAGTTCAGACCCAAGGGCAAGCCCTGTCCTAAAGAGATCCCCAAAGAGTCC |
| | | AAGAACACCGAGGTGCTCGTGTGGGAAGAGTGCGTGGCCAATTCTGCCGTGATCCTGCAGA |
| | | ACAACGAGTTCGGCACCATCATCGACTGGGCCCCTAGAGGCCAGTTCTACCACAATTGCAG |
| | | CGGCCAGACACAGAGCTGCCCTAGCGCTCAAGTGTCACCAGCCGTGGATAGCGATCTGACC |
| | | GAGAGCCTGGACAAGCACAAACACAAGAAGCTGCAGAGCTTCTACCCCTGGGAGTGGGGCG |
| | | AGAAGGGCATCTCTACACCCAGACCTAAGATCGTGTCACCCGTGTCCGGACCTGAGCACCC |
| | | TGAACTTTGGAGACTGACAGTGGCCAGCCACCACATCAGAATTTGGAGCGGCAATCAGACC |
| | | CTGGAAACCCGGGACAGAAAGCCCTTCTACACCGTGGACCTGAACAGCAGCCTGACCGTGC |
| | | CTCTCCAGAGCTGTGTGAAGCCTCCTTACATGCTGGTCGTGGGCAACATCGTGATCAAGCC |
| | | CGACAGCCAGACCATCACATGCGAGAACTGCAGACTGCTGACCTGCATCGACAGCACCTTC |
| | | AACTGGCAGCACCGGATCCTGCTCGTGCGAGCTAGAGAAGGCGTGTGGATCCCCGTGTCTA |
| | | TGGACAGACCTTGGGAAGCTAGCCCCAGCGTGCACATCCTGACAGAGGTGCTGAAGGGCGT |
| | | GCTGAACAGAAGCAAGCGGTTCATCTTCACCCTGATCGCCGTGATCATGGGCCTGATTGCT |
| | | GTGACAGCCACAGCTGCTGTTGCTGGCGTGGCCCTGCATAGCTCTGTGCAGAGCGTGAACT |
| | | TCGTGAACGATTGGCAGAAGAACAGCACCCGGCTGTGGAACAGCCAGAGCAGCATCGATCA |
| | | GAAGCTGGCCAACGCCATCAACGACCTGCGGCAGACAGTGATCTGGATGGGCGACAGACTG |
| | | ATGAGCCTGGAACACCGGTTCCAGCTGCAGTGCGACTGGAATACCAGCGACTTCTGCATCA |
| | | CCCCTCAGATCTACAACGAGAGCGAGCACCACTGGGATATGGTCCGAAGGCATCTGCAGGG |
| | | CAGAGAGGACAACCTGACACTGGACATCAGCAAGCTGAAAGAGCAGATCTTCGAGGCCAGC |
| | | AAGGCCCACCTGAATCTGGTGCCTGGAACCGAAGCTATTGCCGGCGTTGCAGATGGCCTGG |
| | | CCAATCTGAATCCTGTGACCTGGGTCAAGACCATCGGCAGCACCACAATCATCAACCTGAT |
| | | CCTGATCCTCGTGTGCCTGTTCTGCCTGCTGCTTGTGTGCAGATGCACCCAGCAGCTGAGA |
| | | AGAGACAGCGACCATAGAGAACGGGCCATGATGACCATGGCCGTGCTGAGCAAGAGAAAAG |
| | | GCGGCAACGTGGGCAAGAGCAAGCGGGATCAGATCGTGACCGTGTCCGTGTGATGAGCTCG |
| | | CTTTCTTGCTGTCCAATTTCTATTAAAGGTTCCTTTGTTCCCTAAGTCCAACTACTAAACT |
| | | GGGGGATATTATGAAGGGCCTTGAGCATCTGGATTCTGCCTAATAAAAAACATTTATTTTC |
| | | ATTGCAAAAAAAAAAAAAAAAAAAAAAAAAAAAAAAAAAAAAAAAAAAAAAAAAAAAAAAA |
| | | AAAAAAAAAAAAAA |
| 6 | HERV-K | ATGGGCCAGACCAAGAGCAAGATCAAGTCTAAGTACGCCAGCTACCTGAGCTTCATCAAGA |
| | ISDmut | TCCTGCTGAAGCGCGGAGGCGTGAAGGTGTCCACCAAGAACCTGATCAAGCTGTTCCAGAT |
| | Gag Env | CATCGAGCAGTTCTGCCCCTGGTTTCCTGAGCAGGGCACCCTGGATCTGAAGGACTGGAAG |
| | codon | CGGATCGGCAAAGAGCTGAAGCAGGCCGGCAGAAAGGGCAACATCATCCCTCTGACCGTGT |
| | optimized | GGAACGACTGGGCCATCATTAAGGCCGCTCTGGAACCCTTCCAGACCGAAGAGGATAGCGT |
| | by | GTCCGTGTCTGATGCCCCTGGCAGCTGCATCATCGACTGCAACGAGAACACCCGGAAGAAG |
| | Genscript | TCCCAGAAAGAGACAGAGGGCCTGCACTGCGAGTATGTGGCCGAACCTGTGATGGCTCAGA |
| | | GCACCCAGAACGTGGACTACAACCAGCTGCAAGAAGTGATCTACCCCGAGACACTGAAGCT |
| | | GGAAGGCAAGGGCCCTGAACTCGTGGGCCCTTCTGAGTCTAAGCCCAGAGGCACATCTCCT |
| | | CTGCCTGCTGGACAGGTGCCAGTGACACTGCAGCCCCAGAAACAAGTGAAAGAGAACAAGA |
| | | CCCAGCCTCCTGTGGCCTACCAGTATTGGCCTCCAGCCGAGCTGCAGTACAGACCTCCTCC |
| | | AGAGAGCCAGTACGGCTACCCTGGAATGCCTCCTGCTCCTCAAGGCAGAGCCCCTTATCCT |
| | | CAGCCTCCTACCAGACGGCTGAACCCTACAGCTCCTCCTAGCAGACAGGGCTCTGAGCTGC |
| | | ACGAGATCATTGACAAGAGCCGGAAAGAGGGCGACACCGAGGCTTGGCAGTTTCCCGTTAC |
| | | ACTGGAACCCATGCCTCCAGGCGAAGGCGCTCAAGAAGGCGAACCTCCTACAGTGGAAGCC |
| | | CGGTACAAGAGCTTCAGCATCAAAATGCTGAAGGACATGAAGGAAGGCGTCAAGCAGTACG |
| | | GCCCTAACAGCCCCTACATGCGGACCCTGCTGGATTCTATTGCCCACGGCCACCGGCTGAT |
| | | CCCTTACGATTGGGAGATCCTGGCCAAGTCCTCTCTGAGCCCTAGCCAGTTCCTGCAGTTC |
| | | AAGACCTGGTGGATCGACGGCGTGCAAGAACAAGTGCGGCGGAACAGAGCCGCCAATCCTC |
| | | CTGTGAACATCGACGCCGATCAGCTGCTCGGAATCGGCCAGAATTGGAGCACCATCTCTCA |
| | | GCAGGCCCTGATGCAGAACGAGGCCATTGAACAAGTCCGGGCCATCTGCCTGAGAGCCTGG |
| | | GAGAAGATTCAGGACCCCGGCAGCACATGCCCCAGCTTTAATACCGTTCGGCAGGGCAGCA |
| | | AAGAGCCCTATCCTGACTTTGTGGCCCGGCTGCAGGATGTGGCCCAGAAGTCTATTGCCGA |
| | | CGAGAAGGCCCGGAAAGTGATCGTGGAACTGATGGCCTACGAGAACGCCAATCCAGAGTGC |
| | | CAGAGCGCCATCAAGCCCCTGAAGGGAAAAGTGCCTGCCGGCTCCGATGTGATCAGCGAGT |
| | | ACGTGAAGGCCTGCGACGGAATTGGCGGAGCCATGCACAAAGCCATGCTGATGGCACAGGC |
| | | CATCACTGGCGTTGTGCTCGGAGGACAAGTTCGGACCTTTGGCGGCAAGTGCTACAACTGT |
| | | GGCCAGATCGGACACCTGAAGAAAAACTGCCCCGTGCTGAACAAGCAGAACATCACCATCC |
| | | AGGCCACCACCACCGGCAGAGAACCTCCAGATCTGTGCCCTAGATGCAAGAAGGGCAAGCA |
| | | CTGGGCCAGCCAGTGCAGAAGCAAGTTCGACAAGAACGGCCAGCCTCTGAGCGGCAACGAG |
| | | CAAAGAGGACAGCCTCAGGCTCCTCAGCAGACCGGCGCATTTCCAATCCAGCCTTTCGTGC |
| | | CCCAAGGCTTCCAGGGACAACAGCCTCCACTGAGCCAAGTGTTCCAGGGCATTAGCCAGCT |
| | | GCCTCAGTACAACAACTGCCCTCCACCTCAGGCTGCCGTGCAGCAGGGATCAGGCGCTACA |
| | | AATTTTAGCCTGCTGAAGCAGGCAGGGGATGTGGAGGAAAACCCCGGACCTATGAACCCCA |
| | | GCGAGATGCAGAGAAAGGCCCCACCTAGACGGCGGAGACACAGAAATAGAGCCCCTCTGAC |
| | | ACACAAGATGAACAAGATGGTCACCAGCGAGGAACAGATGAAGCTGCCCAGCACCAAGAAG |
| | | GCCGAGCCTCCAACATGGGCCCAGCTGAAGAAACTGACCCAGCTGGCCACCAAGTACCTGG |
| | | AAAACACCAAAGTGACCCAGACACCTGAGAGCATGCTGCTGGCCGCTCTGATGATCGTGTC |
| | | CATGGTGGTGTCCCTGCCTATGCCTGCTGGTGCCGCCGCTGCCAATTACACATACTGGGCC |
| | | TACGTGCCCTTTCCTCCTCTGATTCGGGCCGTGACCTGGATGGACAACCCCATCGAAGTGT |
| | | ACGTGAACGACAGCGTGTGGGTGCCCGGACCAATCGACGATAGATGTCCCGCCAAGCCTGA |
| | | GGAAGAGGGCATGATGATCAACATCAGCATCGGCTACCGGTATCCTCCAATCTGCCTGGGC |
| | | AGAGCCCCTGGCTGTCTTATGCCAGCTGTGCAGAACTGGCTGGTGGAAGTGCCTACCGTGT |
| | | CTCCCATCAGCCGGTTCACCTACCACATGGTGTCCGGCATGAGCCTGCGGCCTAGAGTGAA |
| | | CTACCTGCAGGACTTCAGCTACCAGCGGAGCCTGAAGTTCAGACCCAAGGGCAAGCCCTGT |
| | | CCTAAAGAGATCCCCAAAGAGTCCAAGAACACCGAGGTGCTCGTGTGGGAAGAGTGCGTGG |
| | | CCAATTCTGCCGTGATCCTGCAGAACAACGAGTTCGGCACCATCATCGACTGGGCCCCTAG |
| | | AGGCCAGTTCTACCACAATTGCAGCGGCCAGACACAGAGCTGCCCTAGCGCTCAAGTGTCA |
| | | CCAGCCGTGGATAGCGATCTGACCGAGAGCCTGGACAAGCACAAACACAAGAAGCTGCAGA |
| | | GCTTCTACCCCTGGGAGTGGGGCGAGAAGGGCATCTCTACACCCAGACCTAAGATCGTGTC |
| | | ACCCGTGTCCGGACCTGAGCACCCTGAACTTTGGAGACTGACAGTGGCCAGCCACCACATC |
| | | AGAATTTGGAGCGGCAATCAGACCCTGGAAACCCGGGACAGAAAGCCCTTCTACACCGTGG |
| | | ACCTGAACAGCAGCCTGACCGTGCCTCTCCAGAGCTGTGTGAAGCCTCCTTACATGCTGGT |
| | | CGTGGGCAACATCGTGATCAAGCCCGACAGCCAGACCATCACATGCGAGAACTGCAGACTG |
| | | CTGACCTGCATCGACAGCACCTTCAACTGGCAGCACCGGATCCTGCTCGTGCGAGCTAGAG |
| | | AAGGCGTGTGGATCCCCGTGTCTATGGACAGACCTTGGGAAGCTAGCCCCAGCGTGCACAT |
| | | CCTGACAGAGGTGCTGAAGGGCGTGCTGAACAGAAGCAAGCGGTTCATCTTCACCCTGATC |
| | | GCCGTGATCATGGGCCTGATTGCTGTGACAGCCACAGCTGCTGTTGCTGGCGTGGCCCTGC |
| | | ATAGCTCTGTGCAGAGCGTGAACTTCGTGAACGATTGGCAGAAGAACAGCACCCGGCTGTG |
| | | GAACAGCCAGAGCAGCATCGATCAGAAGCTGGCCAACGCCATCAACGACCTGCGGCAGACA |
| | | GTGATCTGGATGGGCGACAGACTGATGAGCCTGGAACACCGGTTCCAGCTGCAGTGCGACT |
| | | GGAATACCAGCGACTTCTGCATCACCCCTCAGATCTACAACGAGAGCGAGCACCACTGGGA |
| | | TATGGTCCGAAGGCATCTGCAGGGCAGAGAGGACAACCTGACACTGGACATCAGCAAGCTG |
| | | AAAGAGCAGATCTTCGAGGCCAGCAAGGCCCACCTGAATCTGGTGCCTGGAACCGAAGCTA |
| | | TTGCCGGCGTTGCAGATGGCCTGGCCAATCTGAATCCTGTGACCTGGGTCAAGACCATCGG |
| | | CAGCACCACAATCATCAACCTGATCCTGATCCTCGTGTGCCTGTTCTGCCTGCTGCTTGTG |
| | | TGCAGATGCACCCAGCAGCTGAGAAGAGACAGCGACCATAGAGAACGGGCCATGATGACCA |
| | | TGGCCGTGCTGAGCAAGAGAAAAGGCGGCAACGTGGGCAAGAGCAAGCGGGATCAGATCGT |
| | | GACCGTGTCCGTGTGA |
| 7 | HERV-K | ATGGGCCAGACCAAGAGCAAGATCAAGTCTAAGTACGCCAGCTACCTGAGCTTCATCAAGA |
| | Gag Env | TCCTGCTGAAGCGCGGAGGCGTGAAGGTGTCCACCAAGAACCTGATCAAGCTGTTCCAGAT |
| | WT ISD | CATCGAGCAGTTCTGCCCCTGGTTTCCTGAGCAGGGCACCCTGGATCTGAAGGACTGGAAG |
| | codon | CGGATCGGCAAAGAGCTGAAGCAGGCCGGCAGAAAGGGCAACATCATCCCTCTGACCGTGT |
| | optimized | GGAACGACTGGGCCATCATTAAGGCCGCTCTGGAACCCTTCCAGACCGAAGAGGATAGCGT |
| | by | GTCCGTGTCTGATGCCCCTGGCAGCTGCATCATCGACTGCAACGAGAACACCCGGAAGAAG |
| | Genscript | TCCCAGAAAGAGACAGAGGGCCTGCACTGCGAGTATGTGGCCGAACCTGTGATGGCTCAGA |
| | | GCACCCAGAACGTGGACTACAACCAGCTGCAAGAAGTGATCTACCCCGAGACACTGAAGCT |
| | | GGAAGGCAAGGGCCCTGAACTCGTGGGCCCTTCTGAGTCTAAGCCCAGAGGCACATCTCCT |
| | | CTGCCTGCTGGACAGGTGCCAGTGACACTGCAGCCCCAGAAACAAGTGAAAGAGAACAAGA |
| | | CCCAGCCTCCTGTGGCCTACCAGTATTGGCCTCCAGCCGAGCTGCAGTACAGACCTCCTCC |
| | | AGAGAGCCAGTACGGCTACCCTGGAATGCCTCCTGCTCCTCAAGGCAGAGCCCCTTATCCT |
| | | CAGCCTCCTACCAGACGGCTGAACCCTACAGCTCCTCCTAGCAGACAGGGCTCTGAGCTGC |
| | | ACGAGATCATTGACAAGAGCCGGAAAGAGGGCGACACCGAGGCTTGGCAGTTTCCCGTTAC |
| | | ACTGGAACCCATGCCTCCAGGCGAAGGCGCTCAAGAAGGCGAACCTCCTACAGTGGAAGCC |
| | | CGGTACAAGAGCTTCAGCATCAAAATGCTGAAGGACATGAAGGAAGGCGTCAAGCAGTACG |
| | | GCCCTAACAGCCCCTACATGCGGACCCTGCTGGATTCTATTGCCCACGGCCACCGGCTGAT |
| | | CCCTTACGATTGGGAGATCCTGGCCAAGTCCTCTCTGAGCCCTAGCCAGTTCCTGCAGTTC |
| | | AAGACCTGGTGGATCGACGGCGTGCAAGAACAAGTGCGGCGGAACAGAGCCGCCAATCCTC |
| | | CTGTGAACATCGACGCCGATCAGCTGCTCGGAATCGGCCAGAATTGGAGCACCATCTCTCA |
| | | GCAGGCCCTGATGCAGAACGAGGCCATTGAACAAGTCCGGGCCATCTGCCTGAGAGCCTGG |
| | | GAGAAGATTCAGGACCCCGGCAGCACATGCCCCAGCTTTAATACCGTTCGGCAGGGCAGCA |
| | | AAGAGCCCTATCCTGACTTTGTGGCCCGGCTGCAGGATGTGGCCCAGAAGTCTATTGCCGA |
| | | CGAGAAGGCCCGGAAAGTGATCGTGGAACTGATGGCCTACGAGAACGCCAATCCAGAGTGC |
| | | CAGAGCGCCATCAAGCCCCTGAAGGGAAAAGTGCCTGCCGGCTCCGATGTGATCAGCGAGT |
| | | ACGTGAAGGCCTGCGACGGAATTGGCGGAGCCATGCACAAAGCCATGCTGATGGCACAGGC |
| | | CATCACTGGCGTTGTGCTCGGAGGACAAGTTCGGACCTTTGGCGGCAAGTGCTACAACTGT |
| | | GGCCAGATCGGACACCTGAAGAAAAACTGCCCCGTGCTGAACAAGCAGAACATCACCATCC |
| | | AGGCCACCACCACCGGCAGAGAACCTCCAGATCTGTGCCCTAGATGCAAGAAGGGCAAGCA |
| | | CTGGGCCAGCCAGTGCAGAAGCAAGTTCGACAAGAACGGCCAGCCTCTGAGCGGCAACGAG |
| | | CAAAGAGGACAGCCTCAGGCTCCTCAGCAGACCGGCGCATTTCCAATCCAGCCTTTCGTGC |
| | | CCCAAGGCTTCCAGGGACAACAGCCTCCACTGAGCCAAGTGTTCCAGGGCATTAGCCAGCT |
| | | GCCTCAGTACAACAACTGCCCTCCACCTCAGGCTGCCGTGCAGCAGGGATCAGGCGCTACA |
| | | AATTTTAGCCTGCTGAAGCAGGCAGGGGATGTGGAGGAAAACCCCGGACCTATGAACCCCA |
| | | GCGAGATGCAGAGAAAGGCCCCACCTAGACGGCGGAGACACAGAAATAGAGCCCCTCTGAC |
| | | ACACAAGATGAACAAGATGGTCACCAGCGAGGAACAGATGAAGCTGCCCAGCACCAAGAAG |
| | | GCCGAGCCTCCAACATGGGCCCAGCTGAAGAAACTGACCCAGCTGGCCACCAAGTACCTGG |
| | | AAAACACCAAAGTGACCCAGACACCTGAGAGCATGCTGCTGGCCGCTCTGATGATCGTGTC |
| | | CATGGTGGTGTCCCTGCCTATGCCTGCTGGTGCCGCCGCTGCCAATTACACATACTGGGCC |
| | | TACGTGCCCTTTCCTCCTCTGATTCGGGCCGTGACCTGGATGGACAACCCCATCGAAGTGT |
| | | ACGTGAACGACAGCGTGTGGGTGCCCGGACCAATCGACGATAGATGTCCCGCCAAGCCTGA |
| | | GGAAGAGGGCATGATGATCAACATCAGCATCGGCTACCGGTATCCTCCAATCTGCCTGGGC |
| | | AGAGCCCCTGGCTGTCTTATGCCAGCTGTGCAGAACTGGCTGGTGGAAGTGCCTACCGTGT |
| | | CTCCCATCAGCCGGTTCACCTACCACATGGTGTCCGGCATGAGCCTGCGGCCTAGAGTGAA |
| | | CTACCTGCAGGACTTCAGCTACCAGCGGAGCCTGAAGTTCAGACCCAAGGGCAAGCCCTGT |
| | | CCTAAAGAGATCCCCAAAGAGTCCAAGAACACCGAGGTGCTCGTGTGGGAAGAGTGCGTGG |
| | | CCAATTCTGCCGTGATCCTGCAGAACAACGAGTTCGGCACCATCATCGACTGGGCCCCTAG |
| | | AGGCCAGTTCTACCACAATTGCAGCGGCCAGACACAGAGCTGCCCTAGCGCTCAAGTGTCA |
| | | CCAGCCGTGGATAGCGATCTGACCGAGAGCCTGGACAAGCACAAACACAAGAAGCTGCAGA |
| | | GCTTCTACCCCTGGGAGTGGGGCGAGAAGGGCATCTCTACACCCAGACCTAAGATCGTGTC |
| | | ACCCGTGTCCGGACCTGAGCACCCTGAACTTTGGAGACTGACAGTGGCCAGCCACCACATC |
| | | AGAATTTGGAGCGGCAATCAGACCCTGGAAACCCGGGACAGAAAGCCCTTCTACACCGTGG |
| | | ACCTGAACAGCAGCCTGACCGTGCCTCTCCAGAGCTGTGTGAAGCCTCCTTACATGCTGGT |
| | | CGTGGGCAACATCGTGATCAAGCCCGACAGCCAGACCATCACATGCGAGAACTGCAGACTG |
| | | CTGACCTGCATCGACAGCACCTTCAACTGGCAGCACCGGATCCTGCTCGTGCGAGCTAGAG |
| | | AAGGCGTGTGGATCCCCGTGTCTATGGACAGACCTTGGGAAGCTAGCCCCAGCGTGCACAT |
| | | CCTGACAGAGGTGCTGAAGGGCGTGCTGAACAGAAGCAAGCGGTTCATCTTCACCCTGATC |
| | | GCCGTGATCATGGGCCTGATTGCTGTGACAGCCACAGCTGCTGTTGCTGGCGTGGCCCTGC |
| | | ATAGCTCTGTGCAGAGCGTGAACTTCGTGAACGATTGGCAGAAGAACAGCACCCGGCTGTG |
| | | GAACAGCCAGAGCAGCATCGATCAGAAGCTGGCCAACCAGATCAACGACCTGCGGCAGACA |
| | | GTGATCTGGATGGGCGACAGACTGATGAGCCTGGAACACCGGTTCCAGCTGCAGTGCGACT |
| | | GGAATACCAGCGACTTCTGCATCACCCCTCAGATCTACAACGAGAGCGAGCACCACTGGGA |
| | | TATGGTCCGAAGGCATCTGCAGGGCAGAGAGGACAACCTGACACTGGACATCAGCAAGCTG |
| | | AAAGAGCAGATCTTCGAGGCCAGCAAGGCCCACCTGAATCTGGTGCCTGGAACCGAAGCTA |
| | | TTGCCGGCGTTGCAGATGGCCTGGCCAATCTGAATCCTGTGACCTGGGTCAAGACCATCGG |
| | | CAGCACCACAATCATCAACCTGATCCTGATCCTCGTGTGCCTGTTCTGCCTGCTGCTTGTG |
| | | TGCAGATGCACCCAGCAGCTGAGAAGAGACAGCGACCATAGAGAACGGGCCATGATGACCA |
| | | TGGCCGTGCTGAGCAAGAGAAAAGGCGGCAACGTGGGCAAGAGCAAGCGGGATCAGATCGT |
| | | GACCGTGTCCGTGTGA |

TABLE 3
Peptide sequences of different mutated
ISD variants and HERV-K Env and different
mutated ISD variants

| SEQ | | |
|---|---|---|
| ID | | |
| NO: | Description | Sequence |
| 8 | ISDmut of | AANQINDLRQTVIW |
| | HERV-K | |
| | Env, | |
| | mutation | |
| | L522A | |
| 9 | ISDmut of | LQNQINDLRQTVIW |
| | HERV-K | |
| | Env, | |
| | mutation | |
| | A523Q | |
| 10 | ISDmut of | LAAQINDLRQTVIW |
| | HERV-K | |
| | Env, | |
| | mutation | |
| | N524A | |
| 4 | ISDmut of | LANAINDLRQTVIW |
| | HERV-K | |
| | Env, | |
| | mutation | |
| | Q525A | |
| 11 | ISDmut of | LANQANDLRQTVIW |
| | HERV-K | |
| | Env, | |
| | mutation | |
| | I526A | |
| 12 | ISDmut of | LANQIADLRQTVIW |
| | HERV-K | |
| | Env, | |
| | mutation | |
| | N527A | |
| 13 | ISDmut of | LANQINALRQTVIW |
| | HERV-K | |
| | Env, | |
| | mutation | |
| | D528A | |
| 14 | ISDmut of | LANQINDARQTVIW |
| | HERV-K | |
| | Env, | |
| | mutation | |
| | L529A | |
| 15 | ISDmut of | LANQINDLAQTVIW |
| | HERV-K | |
| | Env, | |
| | mutation | |
| | R530A | |
| 16 | ISDmut of | LANQINDLRATVIW |
| | HERV-K | |
| | Env, | |
| | mutation | |
| | Q531A | |
| 17 | ISDmut of | LANQINDLRQAVIW |
| | HERV-K | |
| | Env, | |
| | mutation | |
| | T532A | |
| 18 | ISDmut of | LANQINDLRQTAIW |
| | HERV-K | |
| | Env, | |
| | mutation | |
| | V533A | |
| 19 | ISDmut of | LANQINDLRQTVAW |
| | HERV-K | |
| | Env, | |
| | mutation | |
| | I534A | |
| 20 | ISDmut of | LANQINDLRQTVIA |
| | HERV-K | |
| | Env, | |
| | mutation | |
| | W535A | |

TABLE 4
Peptide sequences of HERV-K Env with different ISD variants and stabilizing
mutation S190C

| SEQ | | |
|---|---|---|
| ID | | |
| NO: | Description | Sequence |
| 21 | HERV-K | MNPSEMQRKAPPRRRRHRNRAPLTHKMNKMVTSEEQMKLPSTKKAEPPTWAQLKKLTQLAT |
| | Env with | KYLENTKVTQTPESMLLAALMIVSMVVSLPMPAGAAAANYTYWAYVPFPPLIRAVTWMDNP |
| | stabilizing | IEVYVNDSVWVPGPIDDRCPAKPEEEGMMINISIGYRYPPICLGRAPGCLMPAVQNWLVEV |
| | mutation | PTVSPICRFTYHMVSGMSLRPRVNYLQDFSYQRSLKFRPKGKPCPKEIPKESKNTEVLVWE |
| | S190C (WT | ECVANSAVILQNNEFGTIIDWAPRGQFYHNCSGQTQSCPSAQVSPAVDSDLTESLDKHKHK |
| | ISD) | KLQSFYPWEWGEKGISTPRPKIVSPVSGPEHPELWRLTVASHHIRIWSGNQTLETRDRKPF |
| | | YTVDLNSSLTVPLQSCVKPPYMLVVGNIVIKPDSQTITCENCRLLTCIDSTENWQHRILLV |
| | | RAREGVWIPVSMDRPWEASPSVHILTEVLKGVLNRSKRFIFTLIAVIMGLIAVTATAAVAG |
| | | VALHSSVQSVNFVNDWQKNSTRLWNSQSSIDQKLANQINDLRQTVIWMGDRLMSLEHRFQL |
| | | QCDWNTSDFCITPQIYNESEHHWDMVRRHLQGREDNLTLDISKLKEQIFEASKAHLNLVPG |
| | | TEAIAGVADGLANLNPVTWVKTIGSTTIINLILILVCLFCLLLVCRCTQQLRRDSDHRERA |
| | | MMTMAVLSKRKGGNVGKSKRDQIVTVSV |
| 22 | HERV-K | MNPSEMQRKAPPRRRRHRNRAPLTHKMNKMVTSEEQMKLPSTKKAEPPTWAQLKKLTQLAT |
| | Env with | KYLENTKVTQTPESMLLAALMIVSMVVSLPMPAGAAAANYTYWAYVPFPPLIRAVTWMDNP |
| | stabilizing | IEVYVNDSVWVPGPIDDRCPAKPEEEGMMINISIGYRYPPICLGRAPGCLMPAVQNWLVEV |
| | mutation | PTVSPICRFTYHMVSGMSLRPRVNYLQDFSYQRSLKFRPKGKPCPKEIPKESKNTEVLVWE |
| | S190C and | ECVANSAVILQNNEFGTIIDWAPRGQFYHNCSGQTQSCPSAQVSPAVDSDLTESLDKHKHK |
| | ISDmut | KLQSFYPWEWGEKGISTPRPKIVSPVSGPEHPELWRLTVASHHIRIWSGNQTLETRDRKPF |
| | with | YTVDLNSSLTVPLQSCVKPPYMLVVGNIVIKPDSQTITCENCRLLTCIDSTENWQHRILLV |
| | mutation | RAREGVWIPVSMDRPWEASPSVHILTEVLKGVLNRSKRFIFTLIAVIMGLIAVTATAAVAG |
| | L522A | VALHSSVQSVNFVNDWQKNSTRLWNSQSSIDQKAANQINDLRQTVIWMGDRLMSLEHRFQL |
| | | QCDWNTSDFCITPQIYNESEHHWDMVRRHLQGREDNLTLDISKLKEQIFEASKAHLNLVPG |
| | | TEAIAGVADGLANLNPVTWVKTIGSTTIINLILILVCLFCLLLVCRCTQQLRRDSDHRERA |
| | | MMTMAVLSKRKGGNVGKSKRDQIVTVSV |
| 23 | HERV-K | MNPSEMQRKAPPRRRRHRNRAPLTHKMNKMVTSEEQMKLPSTKKAEPPTWAQLKKLTQLAT |
| | Env with | KYLENTKVTQTPESMLLAALMIVSMVVSLPMPAGAAAANYTYWAYVPFPPLIRAVTWMDNP |
| | stabilizing | IEVYVNDSVWVPGPIDDRCPAKPEEEGMMINISIGYRYPPICLGRAPGCLMPAVQNWLVEV |
| | mutation | PTVSPICRFTYHMVSGMSLRPRVNYLQDFSYQRSLKFRPKGKPCPKEIPKESKNTEVLVWE |
| | S190C and | ECVANSAVILQNNEFGTIIDWAPRGQFYHNCSGQTQSCPSAQVSPAVDSDLTESLDKHKHK |
| | ISDmut | KLQSFYPWEWGEKGISTPRPKIVSPVSGPEHPELWRLTVASHHIRIWSGNQTLETRDRKPF |
| | with | YTVDLNSSLTVPLQSCVKPPYMLVVGNIVIKPDSQTITCENCRLLTCIDSTENWQHRILLV |
| | mutation | RAREGVWIPVSMDRPWEASPSVHILTEVLKGVLNRSKRFIFTLIAVIMGLIAVTATAAVAG |
| | A523Q | VALHSSVQSVNFVNDWQKNSTRLWNSQSSIDQKLQNQINDLRQTVIWMGDRLMSLEHRFQL |
| | | QCDWNTSDFCITPQIYNESEHHWDMVRRHLQGREDNLTLDISKLKEQIFEASKAHLNLVPG |
| | | TEAIAGVADGLANLNPVTWVKTIGSTTIINLILILVCLFCLLLVCRCTQQLRRDSDHRERA |
| | | MMTMAVLSKRKGGNVGKSKRDQIVTVSV |
| 24 | HERV-K | MNPSEMQRKAPPRRRRHRNRAPLTHKMNKMVTSEEQMKLPSTKKAEPPTWAQLKKLTQLAT |
| | Env with | KYLENTKVTQTPESMLLAALMIVSMVVSLPMPAGAAAANYTYWAYVPFPPLIRAVTWMDNP |
| | stabilizing | IEVYVNDSVWVPGPIDDRCPAKPEEEGMMINISIGYRYPPICLGRAPGCLMPAVQNWLVEV |
| | mutation | PTVSPICRFTYHMVSGMSLRPRVNYLQDFSYQRSLKFRPKGKPCPKEIPKESKNTEVLVWE |
| | S190C and | ECVANSAVILQNNEFGTIIDWAPRGQFYHNCSGQTQSCPSAQVSPAVDSDLTESLDKHKHK |
| | ISDmut | KLQSFYPWEWGEKGISTPRPKIVSPVSGPEHPELWRLTVASHHIRIWSGNQTLETRDRKPF |
| | with | YTVDLNSSLTVPLQSCVKPPYMLVVGNIVIKPDSQTITCENCRLLTCIDSTENWQHRILLV |
| | mutation | RAREGVWIPVSMDRPWEASPSVHILTEVLKGVLNRSKRFIFTLIAVIMGLIAVTATAAVAG |
| | N524A | VALHSSVQSVNFVNDWQKNSTRLWNSQSSIDQKLAAQINDLRQTVIWMGDRLMSLEHRFQL |
| | | QCDWNTSDFCITPQIYNESEHHWDMVRRHLQGREDNLTLDISKLKEQIFEASKAHLNLVPG |
| | | TEAIAGVADGLANLNPVTWVKTIGSTTIINLILILVCLFCLLLVCRCTQQLRRDSDHRERA |
| | | MMTMAVLSKRKGGNVGKSKRDQIVTVSV |
| 25 | HERV-K | MNPSEMQRKAPPRRRRHRNRAPLTHKMNKMVTSEEQMKLPSTKKAEPPTWAQLKKLTQLAT |
| | Env with | KYLENTKVTQTPESMLLAALMIVSMVVSLPMPAGAAAANYTYWAYVPFPPLIRAVTWMDNP |
| | stabilizing | IEVYVNDSVWVPGPIDDRCPAKPEEEGMMINISIGYRYPPICLGRAPGCLMPAVQNWLVEV |
| | mutation | PTVSPICRFTYHMVSGMSLRPRVNYLQDFSYQRSLKFRPKGKPCPKEIPKESKNTEVLVWE |
| | S190C and | ECVANSAVILQNNEFGTIIDWAPRGQFYHNCSGQTQSCPSAQVSPAVDSDLTESLDKHKHK |
| | ISDmut | KLQSFYPWEWGEKGISTPRPKIVSPVSGPEHPELWRLTVASHHIRIWSGNQTLETRDRKPF |
| | with | YTVDLNSSLTVPLQSCVKPPYMLVVGNIVIKPDSQTITCENCRLLTCIDSTENWQHRILLV |
| | mutation | RAREGVWIPVSMDRPWEASPSVHILTEVLKGVLNRSKRFIFTLIAVIMGLIAVTATAAVAG |
| | Q525A | VALHSSVQSVNFVNDWQKNSTRLWNSQSSIDQKLANAINDLRQTVIWMGDRLMSLEHRFQL |
| | | QCDWNTSDFCITPQIYNESEHHWDMVRRHLQGREDNLILDISKLKEQIFEASKAHLNLVPG |
| | | TEAIAGVADGLANLNPVTWVKTIGSTTIINLILILVCLFCLLLVCRCTQQLRRDSDHRERA |
| | | MMTMAVLSKRKGGNVGKSKRDQIVTVSV |
| 26 | HERV-K | MNPSEMQRKAPPRRRRHRNRAPLTHKMNKMVTSEEQMKLPSTKKAEPPTWAQLKKLTQLAT |
| | Env with | KYLENTKVTQTPESMLLAALMIVSMVVSLPMPAGAAAANYTYWAYVPFPPLIRAVTWMDNP |
| | stabilizing | IEVYVNDSVWVPGPIDDRCPAKPEEEGMMINISIGYRYPPICLGRAPGCLMPAVQNWLVEV |
| | mutation | PTVSPICRFTYHMVSGMSLRPRVNYLQDFSYQRSLKFRPKGKPCPKEIPKESKNTEVLVWE |
| | S190C and | ECVANSAVILQNNEFGTIIDWAPRGQFYHNCSGQTQSCPSAQVSPAVDSDLTESLDKHKHK |
| | ISDmut | KLQSFYPWEWGEKGISTPRPKIVSPVSGPEHPELWRLTVASHHIRIWSGNQTLETRDRKPF |
| | with | YTVDLNSSLTVPLQSCVKPPYMLVVGNIVIKPDSQTITCENCRLLTCIDSTFNWQHRILLV |
| | | RAREGVWIPVSMDRPWEASPSVHILTEVLKGVLNRSKRFIFTLIAVIMGLIAVTATAAVAG |
| | mutation | VALHSSVQSVNFVNDWQKNSTRLWNSQSSIDQKLANQANDLRQTVIWMGDRLMSLEHRFQL |
| | I526A | QCDWNTSDFCITPQIYNESEHHWDMVRRHLQGREDNLTLDISKLKEQIFEASKAHLNLVPG |
| | | TEAIAGVADGLANLNPVTWVKTIGSTTIINLILILVCLFCLLLVCRCTQQLRRDSDHRERA |
| | | MMTMAVLSKRKGGNVGKSKRDQIVTVSV |
| 27 | HERV-K | MNPSEMQRKAPPRRRRHRNRAPLTHKMNKMVTSEEQMKLPSTKKAEPPTWAQLKKLTQLAT |
| | Env with | KYLENTKVTQTPESMLLAALMIVSMVVSLPMPAGAAAANYTYWAYVPFPPLIRAVTWMDNP |
| | stabilizing | IEVYVNDSVWVPGPIDDRCPAKPEEEGMMINISIGYRYPPICLGRAPGCLMPAVQNWLVEV |
| | mutation | PTVSPICRFTYHMVSGMSLRPRVNYLQDFSYQRSLKFRPKGKPCPKEIPKESKNTEVLVWE |
| | S190C and | ECVANSAVILQNNEFGTIIDWAPRGQFYHNCSGQTQSCPSAQVSPAVDSDLTESLDKHKHK |
| | ISDmut | KLQSFYPWEWGEKGISTPRPKIVSPVSGPEHPELWRLTVASHHIRIWSGNQTLETRDRKPF |
| | with | YTVDLNSSLTVPLQSCVKPPYMLVVGNIVIKPDSQTITCENCRLLTCIDSTENWQHRILLV |
| | mutation | RAREGVWIPVSMDRPWEASPSVHILTEVLKGVLNRSKRFIFTLIAVIMGLIAVTATAAVAG |
| | N527A | VALHSSVQSVNFVNDWQKNSTRLWNSQSSIDQKLANQIADLRQTVIWMGDRLMSLEHRFQL |
| | | QCDWNTSDFCITPQIYNESEHHWDMVRRHLQGREDNLTLDISKLKEQIFEASKAHLNLVPG |
| | | TEAIAGVADGLANLNPVTWVKTIGSTTIINLILILVCLFCLLLVCRCTQQLRRDSDHRERA |
| | | MMTMAVLSKRKGGNVGKSKRDQIVTVSV |
| 28 | HERV-K | MNPSEMQRKAPPRRRRHRNRAPLTHKMNKMVTSEEQMKLPSTKKAEPPTWAQLKKLTQLAT |
| | Env with | KYLENTKVTQTPESMLLAALMIVSMVVSLPMPAGAAAANYTYWAYVPFPPLIRAVTWMDNP |
| | stabilizing | IEVYVNDSVWVPGPIDDRCPAKPEEEGMMINISIGYRYPPICLGRAPGCLMPAVQNWLVEV |
| | mutation | PTVSPICRFTYHMVSGMSLRPRVNYLQDFSYQRSLKFRPKGKPCPKEIPKESKNTEVLVWE |
| | S190C and | ECVANSAVILQNNEFGTIIDWAPRGQFYHNCSGQTQSCPSAQVSPAVDSDLTESLDKHKHK |
| | ISDmut | KLQSFYPWEWGEKGISTPRPKIVSPVSGPEHPELWRLTVASHHIRIWSGNQTLETRDRKPF |
| | with | YTVDLNSSLTVPLQSCVKPPYMLVVGNIVIKPDSQTITCENCRLLTCIDSTENWQHRILLV |
| | mutation | RAREGVWIPVSMDRPWEASPSVHILTEVLKGVLNRSKRFIFTLIAVIMGLIAVTATAAVAG |
| | D528A | VALHSSVQSVNFVNDWQKNSTRLWNSQSSIDQKLANQINALRQTVIWMGDRLMSLEHRFQL |
| | | QCDWNTSDFCITPQIYNESEHHWDMVRRHLQGREDNLTLDISKLKEQIFEASKAHLNLVPG |
| | | TEAIAGVADGLANLNPVTWVKTIGSTTIINLILILVCLFCLLLVCRCTQQLRRDSDHRERA |
| | | MMTMAVLSKRKGGNVGKSKRDQIVTVSV |
| 29 | HERV-K | MNPSEMQRKAPPRRRRHRNRAPLTHKMNKMVTSEEQMKLPSTKKAEPPTWAQLKKLTQLAT |
| | Env with | KYLENTKVTQTPESMLLAALMIVSMVVSLPMPAGAAAANYTYWAYVPFPPLIRAVTWMDNP |
| | stabilizing | IEVYVNDSVWVPGPIDDRCPAKPEEEGMMINISIGYRYPPICLGRAPGCLMPAVQNWLVEV |
| | mutation | PTVSPICRFTYHMVSGMSLRPRVNYLQDFSYQRSLKFRPKGKPCPKEIPKESKNTEVLVWE |
| | S190C and | ECVANSAVILQNNEFGTIIDWAPRGQFYHNCSGQTQSCPSAQVSPAVDSDLTESLDKHKHK |
| | ISDmut | KLQSFYPWEWGEKGISTPRPKIVSPVSGPEHPELWRLTVASHHIRIWSGNQTLETRDRKPF |
| | with | YTVDLNSSLTVPLQSCVKPPYMLVVGNIVIKPDSQTITCENCRLLTCIDSTENWQHRILLV |
| | mutation | RAREGVWIPVSMDRPWEASPSVHILTEVLKGVLNRSKRFIFTLIAVIMGLIAVTATAAVAG |
| | L529A | VALHSSVQSVNFVNDWQKNSTRLWNSQSSIDQKLANQINDARQTVIWMGDRLMSLEHRFQL |
| | | QCDWNTSDFCITPQIYNESEHHWDMVRRHLQGREDNLTLDISKLKEQIFEASKAHLNLVPG |
| | | TEAIAGVADGLANLNPVTWVKTIGSTTIINLILILVCLFCLLLVCRCTQQLRRDSDHRERA |
| | | MMTMAVLSKRKGGNVGKSKRDQIVTVSV |
| 30 | HERV-K | MNPSEMQRKAPPRRRRHRNRAPLTHKMNKMVTSEEQMKLPSTKKAEPPTWAQLKKLTQLAT |
| | Env with | KYLENTKVTQTPESMLLAALMIVSMVVSLPMPAGAAAANYTYWAYVPFPPLIRAVTWMDNP |
| | stabilizing | IEVYVNDSVWVPGPIDDRCPAKPEEEGMMINISIGYRYPPICLGRAPGCLMPAVQNWLVEV |
| | mutation | PTVSPICRFTYHMVSGMSLRPRVNYLQDFSYQRSLKFRPKGKPCPKEIPKESKNTEVLVWE |
| | S190C and | ECVANSAVILQNNEFGTIIDWAPRGQFYHNCSGQTQSCPSAQVSPAVDSDLTESLDKHKHK |
| | ISDmut | KLQSFYPWEWGEKGISTPRPKIVSPVSGPEHPELWRLTVASHHIRIWSGNQTLETRDRKPF |
| | with | YTVDLNSSLTVPLQSCVKPPYMLVVGNIVIKPDSQTITCENCRLLTCIDSTENWQHRILLV |
| | mutation | RAREGVWIPVSMDRPWEASPSVHILTEVLKGVLNRSKRFIFTLIAVIMGLIAVTATAAVAG |
| | R530A | VALHSSVQSVNFVNDWQKNSTRLWNSQSSIDQKLANQINDLAQTVIWMGDRLMSLEHRFQL |
| | | QCDWNTSDFCITPQIYNESEHHWDMVRRHLQGREDNLTLDISKLKEQIFEASKAHLNLVPG |
| | | TEAIAGVADGLANLNPVTWVKTIGSTTIINLILILVCLFCLLLVCRCTQQLRRDSDHRERA |
| | | MMTMAVLSKRKGGNVGKSKRDQIVTVSV |
| 31 | HERV-K | MNPSEMQRKAPPRRRRHRNRAPLTHKMNKMVTSEEQMKLPSTKKAEPPTWAQLKKLTQLAT |
| | Env with | KYLENTKVTQTPESMLLAALMIVSMVVSLPMPAGAAAANYTYWAYVPFPPLIRAVTWMDNP |
| | stabilizing | IEVYVNDSVWVPGPIDDRCPAKPEEEGMMINISIGYRYPPICLGRAPGCLMPAVQNWLVEV |
| | mutation | PTVSPICRFTYHMVSGMSLRPRVNYLQDFSYQRSLKFRPKGKPCPKEIPKESKNTEVLVWE |
| | S190C and | ECVANSAVILQNNEFGTIIDWAPRGQFYHNCSGQTQSCPSAQVSPAVDSDLTESLDKHKHK |
| | | KLQSFYPWEWGEKGISTPRPKIVSPVSGPEHPELWRLTVASHHIRIWSGNQTLETRDRKPF |
| | ISDmut | YTVDLNSSLTVPLQSCVKPPYMLVVGNIVIKPDSQTITCENCRLLTCIDSTENWQHRILLV |
| | with | RAREGVWIPVSMDRPWEASPSVHILTEVLKGVLNRSKRFIFTLIAVIMGLIAVTATAAVAG |
| | mutation | VALHSSVQSVNFVNDWQKNSTRLWNSQSSIDQKLANQINDLRATVIWMGDRLMSLEHRFQL |
| | Q531A | QCDWNTSDFCITPQIYNESEHHWDMVRRHLQGREDNLTLDISKLKEQIFEASKAHLNLVPG |
| | | TEAIAGVADGLANLNPVTWVKTIGSTTIINLILILVCLFCLLLVCRCTQQLRRDSDHRERA |
| | | MMTMAVLSKRKGGNVGKSKRDQIVTVSV |
| 32 | HERV-K | MNPSEMQRKAPPRRRRHRNRAPLTHKMNKMVTSEEQMKLPSTKKAEPPTWAQLKKLTQLAT |
| | Env with | KYLENTKVTQTPESMLLAALMIVSMVVSLPMPAGAAAANYTYWAYVPFPPLIRAVTWMDNP |
| | stabilizing | IEVYVNDSVWVPGPIDDRCPAKPEEEGMMINISIGYRYPPICLGRAPGCLMPAVQNWLVEV |
| | mutation | PTVSPICRFTYHMVSGMSLRPRVNYLQDFSYQRSLKFRPKGKPCPKEIPKESKNTEVLVWE |
| | S190C and | ECVANSAVILQNNEFGTIIDWAPRGQFYHNCSGQTQSCPSAQVSPAVDSDLTESLDKHKHK |
| | ISDmut | KLQSFYPWEWGEKGISTPRPKIVSPVSGPEHPELWRLTVASHHIRIWSGNQTLETRDRKPF |
| | with | YTVDLNSSLTVPLQSCVKPPYMLVVGNIVIKPDSQTITCENCRLLTCIDSTENWQHRILLV |
| | mutation | RAREGVWIPVSMDRPWEASPSVHILTEVLKGVLNRSKRFIFTLIAVIMGLIAVTATAAVAG |
| | T532A | VALHSSVQSVNFVNDWQKNSTRLWNSQSSIDQKLANQINDLRQAVIWMGDRLMSLEHRFQL |
| | | QCDWNTSDFCITPQIYNESEHHWDMVRRHLQGREDNLTLDISKLKEQIFEASKAHLNLVPG |
| | | TEAIAGVADGLANLNPVTWVKTIGSTTIINLILILVCLFCLLLVCRCTQQLRRDSDHRERA |
| | | MMTMAVLSKRKGGNVGKSKRDQIVTVSV |
| 33 | HERV-K | MNPSEMQRKAPPRRRRHRNRAPLTHKMNKMVTSEEQMKLPSTKKAEPPTWAQLKKLTQLAT |
| | Env with | KYLENTKVTQTPESMLLAALMIVSMVVSLPMPAGAAAANYTYWAYVPFPPLIRAVTWMDNP |
| | stabilizing | IEVYVNDSVWVPGPIDDRCPAKPEEEGMMINISIGYRYPPICLGRAPGCLMPAVQNWLVEV |
| | mutation | PTVSPICRFTYHMVSGMSLRPRVNYLQDFSYQRSLKFRPKGKPCPKEIPKESKNTEVLVWE |
| | S190C and | ECVANSAVILQNNEFGTIIDWAPRGQFYHNCSGQTQSCPSAQVSPAVDSDLTESLDKHKHK |
| | ISDmut | KLQSFYPWEWGEKGISTPRPKIVSPVSGPEHPELWRLTVASHHIRIWSGNQTLETRDRKPF |
| | with | YTVDLNSSLTVPLQSCVKPPYMLVVGNIVIKPDSQTITCENCRLLTCIDSTENWQHRILLV |
| | mutation | RAREGVWIPVSMDRPWEASPSVHILTEVLKGVLNRSKRFIFTLIAVIMGLIAVTATAAVAG |
| | V533A | VALHSSVQSVNFVNDWQKNSTRLWNSQSSIDQKLANQINDLRQTAIWMGDRLMSLEHRFQL |
| | | QCDWNTSDFCITPQIYNESEHHWDMVRRHLQGREDNLTLDISKLKEQIFEASKAHLNLVPG |
| | | TEAIAGVADGLANLNPVTWVKTIGSTTIINLILILVCLFCLLLVCRCTQQLRRDSDHRERA |
| | | MMTMAVLSKRKGGNVGKSKRDQIVTVSV |
| 34 | HERV-K | MNPSEMQRKAPPRRRRHRNRAPLTHKMNKMVTSEEQMKLPSTKKAEPPTWAQLKKLTQLAT |
| | Env with | KYLENTKVTQTPESMLLAALMIVSMVVSLPMPAGAAAANYTYWAYVPFPPLIRAVTWMDNP |
| | stabilizing | IEVYVNDSVWVPGPIDDRCPAKPEEEGMMINISIGYRYPPICLGRAPGCLMPAVQNWLVEV |
| | mutation | PTVSPICRFTYHMVSGMSLRPRVNYLQDFSYQRSLKFRPKGKPCPKEIPKESKNTEVLVWE |
| | S190C and | ECVANSAVILQNNEFGTIIDWAPRGQFYHNCSGQTQSCPSAQVSPAVDSDLTESLDKHKHK |
| | ISDmut | KLQSFYPWEWGEKGISTPRPKIVSPVSGPEHPELWRLTVASHHIRIWSGNQTLETRDRKPF |
| | with | YTVDLNSSLTVPLQSCVKPPYMLVVGNIVIKPDSQTITCENCRLLTCIDSTENWQHRILLV |
| | mutation | RAREGVWIPVSMDRPWEASPSVHILTEVLKGVLNRSKRFIFTLIAVIMGLIAVTATAAVAG |
| | I534A | VALHSSVQSVNFVNDWQKNSTRLWNSQSSIDQKLANQINDLRQTVAWMGDRLMSLEHRFQL |
| | | QCDWNTSDFCITPQIYNESEHHWDMVRRHLQGREDNLTLDISKLKEQIFEASKAHLNLVPG |
| | | TEAIAGVADGLANLNPVTWVKTIGSTTIINLILILVCLFCLLLVCRCTQQLRRDSDHRERA |
| | | MMTMAVLSKRKGGNVGKSKRDQIVTVSV |
| 35 | HERV-K | MNPSEMQRKAPPRRRRHRNRAPLTHKMNKMVTSEEQMKLPSTKKAEPPTWAQLKKLTQLAT |
| | Env with | KYLENTKVTQTPESMLLAALMIVSMVVSLPMPAGAAAANYTYWAYVPFPPLIRAVTWMDNP |
| | stabilizing | IEVYVNDSVWVPGPIDDRCPAKPEEEGMMINISIGYRYPPICLGRAPGCLMPAVQNWLVEV |
| | mutation | PTVSPICRFTYHMVSGMSLRPRVNYLQDFSYQRSLKFRPKGKPCPKEIPKESKNTEVLVWE |
| | S190C and | ECVANSAVILQNNEFGTIIDWAPRGQFYHNCSGQTQSCPSAQVSPAVDSDLTESLDKHKHK |
| | ISDmut | KLQSFYPWEWGEKGISTPRPKIVSPVSGPEHPELWRLTVASHHIRIWSGNQTLETRDRKPF |
| | with | YTVDLNSSLTVPLQSCVKPPYMLVVGNIVIKPDSQTITCENCRLLTCIDSTENWQHRILLV |
| | mutation | RAREGVWIPVSMDRPWEASPSVHILTEVLKGVLNRSKRFIFTLIAVIMGLIAVTATAAVAG |
| | W535A | VALHSSVQSVNFVNDWQKNSTRLWNSQSSIDQKLANQINDLRQTVIAMGDRLMSLEHRFQL |
| | | QCDWNTSDFCITPQIYNESEHHWDMVRRHLQGREDNLTLDISKLKEQIFEASKAHLNLVPG |
| | | TEAIAGVADGLANLNPVTWVKTIGSTTIINLILILVCLFCLLLVCRCTQQLRRDSDHRERA |
| | | MMTMAVLSKRKGGNVGKSKRDQIVTVSV |

TABLE 5
Nucleotide sequences for mRNA constructs used for transfection/immunization

| SEQ | | |
|---|---|---|
| ID | | |
| NO: | Description | Sequence |
| 36 | 5′UTR | GAATAAACTAGTATTCTTCTGGTCCCCACAGACTCAGAGAGAACCCGCCACCATGGGCCAG |
| | (partial) of | ACCAAGAGCAAGATCAAGTCTAAGTACGCCAGCTACCTGAGCTTCATCAAGATCCTGCTGA |
| | HBA1- | AGCGCGGAGGCGTGAAGGTGTCCACCAAGAACCTGATCAAGCTGTTCCAGATCATCGAGCA |
| | HERV-K | GTTCTGCCCCTGGTTTCCTGAGCAGGGCACCCTGGATCTGAAGGACTGGAAGCGGATCGGC |
| | Gag Env | AAAGAGCTGAAGCAGGCCGGCAGAAAGGGCAACATCATCCCTCTGACCGTGTGGAACGACT |
| | ISDmut | GGGCCATCATTAAGGCCGCTCTGGAACCCTTCCAGACCGAAGAGGATAGCGTGTCCGTGTC |
| | (with ISD | TGATGCCCCTGGCAGCTGCATCATCGACTGCAACGAGAACACCCGGAAGAAGTCCCAGAAA |
| | mutation | GAGACAGAGGGCCTGCACTGCGAGTATGTGGCCGAACCTGTGATGGCTCAGAGCACCCAGA |
| | Q525A)- | ACGTGGACTACAACCAGCTGCAAGAAGTGATCTACCCCGAGACACTGAAGCTGGAAGGCAA |
| | 3′UTR of | GGGCCCTGAACTCGTGGGCCCTTCTGAGTCTAAGCCCAGAGGCACATCTCCTCTGCCTGCT |
| | AES | GGACAGGTGCCAGTGACACTGCAGCCCCAGAAACAAGTGAAAGAGAACAAGACCCAGCCTC |
| | mtRNR1- | CTGTGGCCTACCAGTATTGGCCTCCAGCCGAGCTGCAGTACAGACCTCCTCCAGAGAGCCA |
| | PolyA (30- | GTACGGCTACCCTGGAATGCCTCCTGCTCCTCAAGGCAGAGCCCCTTATCCTCAGCCTCCT |
| | linker-70) | ACCAGACGGCTGAACCCTACAGCTCCTCCTAGCAGACAGGGCTCTGAGCTGCACGAGATCA |
| | = “RNA 4” | TTGACAAGAGCCGGAAAGAGGGCGACACCGAGGCTTGGCAGTTTCCCGTTACACTGGAACC |
| | (sequence | CATGCCTCCAGGCGAAGGCGCTCAAGAAGGCGAACCTCCTACAGTGGAAGCCCGGTACAAG |
| | encoding | AGCTTCAGCATCAAAATGCTGAAGGACATGAAGGAAGGCGTCAAGCAGTACGGCCCTAACA |
| | ISD in bold) | GCCCCTACATGCGGACCCTGCTGGATTCTATTGCCCACGGCCACCGGCTGATCCCTTACGA |
| | | TTGGGAGATCCTGGCCAAGTCCTCTCTGAGCCCTAGCCAGTTCCTGCAGTTCAAGACCTGG |
| | | TGGATCGACGGCGTGCAAGAACAAGTGCGGCGGAACAGAGCCGCCAATCCTCCTGTGAACA |
| | | TCGACGCCGATCAGCTGCTCGGAATCGGCCAGAATTGGAGCACCATCTCTCAGCAGGCCCT |
| | | GATGCAGAACGAGGCCATTGAACAAGTCCGGGCCATCTGCCTGAGAGCCTGGGAGAAGATT |
| | | CAGGACCCCGGCAGCACATGCCCCAGCTTTAATACCGTTCGGCAGGGCAGCAAAGAGCCCT |
| | | ATCCTGACTTTGTGGCCCGGCTGCAGGATGTGGCCCAGAAGTCTATTGCCGACGAGAAGGC |
| | | CCGGAAAGTGATCGTGGAACTGATGGCCTACGAGAACGCCAATCCAGAGTGCCAGAGCGCC |
| | | ATCAAGCCCCTGAAGGGAAAAGTGCCTGCCGGCTCCGATGTGATCAGCGAGTACGTGAAGG |
| | | CCTGCGACGGAATTGGCGGAGCCATGCACAAAGCCATGCTGATGGCACAGGCCATCACTGG |
| | | CGTTGTGCTCGGAGGACAAGTTCGGACCTTTGGCGGCAAGTGCTACAACTGTGGCCAGATC |
| | | GGACACCTGAAGAAAAACTGCCCCGTGCTGAACAAGCAGAACATCACCATCCAGGCCACCA |
| | | CCACCGGCAGAGAACCTCCAGATCTGTGCCCTAGATGCAAGAAGGGCAAGCACTGGGCCAG |
| | | CCAGTGCAGAAGCAAGTTCGACAAGAACGGCCAGCCTCTGAGCGGCAACGAGCAAAGAGGA |
| | | CAGCCTCAGGCTCCTCAGCAGACCGGCGCATTTCCAATCCAGCCTTTCGTGCCCCAAGGCT |
| | | TCCAGGGACAACAGCCTCCACTGAGCCAAGTGTTCCAGGGCATTAGCCAGCTGCCTCAGTA |
| | | CAACAACTGCCCTCCACCTCAGGCTGCCGTGCAGCAGGGATCAGGCGCTACAAATTTTAGC |
| | | CTGCTGAAGCAGGCAGGGGATGTGGAGGAAAACCCCGGACCTATGAACCCCAGCGAGATGC |
| | | AGAGAAAGGCCCCACCTAGACGGCGGAGACACAGAAATAGAGCCCCTCTGACACACAAGAT |
| | | GAACAAGATGGTCACCAGCGAGGAACAGATGAAGCTGCCCAGCACCAAGAAGGCCGAGCCT |
| | | CCAACATGGGCCCAGCTGAAGAAACTGACCCAGCTGGCCACCAAGTACCTGGAAAACACCA |
| | | AAGTGACCCAGACACCTGAGAGCATGCTGCTGGCCGCTCTGATGATCGTGTCCATGGTGGT |
| | | GTCCCTGCCTATGCCTGCTGGTGCCGCCGCTGCCAATTACACATACTGGGCCTACGTGCCC |
| | | TTTCCTCCTCTGATTCGGGCCGTGACCTGGATGGACAACCCCATCGAAGTGTACGTGAACG |
| | | ACAGCGTGTGGGTGCCCGGACCAATCGACGATAGATGTCCCGCCAAGCCTGAGGAAGAGGG |
| | | CATGATGATCAACATCAGCATCGGCTACCGGTATCCTCCAATCTGCCTGGGCAGAGCCCCT |
| | | GGCTGTCTTATGCCAGCTGTGCAGAACTGGCTGGTGGAAGTGCCTACCGTGTCTCCCATCA |
| | | GCCGGTTCACCTACCACATGGTGTCCGGCATGAGCCTGCGGCCTAGAGTGAACTACCTGCA |
| | | GGACTTCAGCTACCAGCGGAGCCTGAAGTTCAGACCCAAGGGCAAGCCCTGTCCTAAAGAG |
| | | ATCCCCAAAGAGTCCAAGAACACCGAGGTGCTCGTGTGGGAAGAGTGCGTGGCCAATTCTG |
| | | CCGTGATCCTGCAGAACAACGAGTTCGGCACCATCATCGACTGGGCCCCTAGAGGCCAGTT |
| | | CTACCACAATTGCAGCGGCCAGACACAGAGCTGCCCTAGCGCTCAAGTGTCACCAGCCGTG |
| | | GATAGCGATCTGACCGAGAGCCTGGACAAGCACAAACACAAGAAGCTGCAGAGCTTCTACC |
| | | CCTGGGAGTGGGGCGAGAAGGGCATCTCTACACCCAGACCTAAGATCGTGTCACCCGTGTC |
| | | CGGACCTGAGCACCCTGAACTTTGGAGACTGACAGTGGCCAGCCACCACATCAGAATTTGG |
| | | AGCGGCAATCAGACCCTGGAAACCCGGGACAGAAAGCCCTTCTACACCGTGGACCTGAACA |
| | | GCAGCCTGACCGTGCCTCTCCAGAGCTGTGTGAAGCCTCCTTACATGCTGGTCGTGGGCAA |
| | | ATCGACAGCACCTTCAACTGGCAGCACCGGATCCTGCTCGTGCGAGCTAGAGAAGGCGTGT |
| | | GGATCCCCGTGTCTATGGACAGACCTTGGGAAGCTAGCCCCAGCGTGCACATCCTGACAGA |
| | | GGTGCTGAAGGGCGTGCTGAACAGAAGCAAGCGGTTCATCTTCACCCTGATCGCCGTGATC |
| | | ATGGGCCTGATTGCTGTGACAGCCACAGCTGCTGTTGCTGGCGTGGCCCTGCATAGCTCTG |
| | | TGCAGAGCGTGAACTTCGTGAACGATTGGCAGAAGAACAGCACCCGGCTGTGGAACAGCCA |
| | | GAGCAGCATCGATCAGAAGCTGGCCAACGCCATCAACGACCTGCGGCAGACAGTGATCTGG |
| | | ATGGGCGACAGACTGATGAGCCTGGAACACCGGTTCCAGCTGCAGTGCGACTGGAATACCA |
| | | GCGACTTCTGCATCACCCCTCAGATCTACAACGAGAGCGAGCACCACTGGGATATGGTCCG |
| | | AAGGCATCTGCAGGGCAGAGAGGACAACCTGACACTGGACATCAGCAAGCTGAAAGAGCAG |
| | | ATCTTCGAGGCCAGCAAGGCCCACCTGAATCTGGTGCCTGGAACCGAAGCTATTGCCGGCG |
| | | TTGCAGATGGCCTGGCCAATCTGAATCCTGTGACCTGGGTCAAGACCATCGGCAGCACCAC |
| | | AATCATCAACCTGATCCTGATCCTCGTGTGCCTGTTCTGCCTGCTGCTTGTGTGCAGATGC |
| | | ACCCAGCAGCTGAGAAGAGACAGCGACCATAGAGAACGGGCCATGATGACCATGGCCGTGC |
| | | TGAGCAAGAGAAAAGGCGGCAACGTGGGCAAGAGCAAGCGGGATCAGATCGTGACCGTGTC |
| | | CGTGTGATGACTCGAGCTGGTACTGCATGCACGCAATGCTAGCTGCCCCTTTCCCGTCCTG |
| | | GGTACCCCGAGTCTCCCCCGACCTCGGGTCCCAGGTATGCTCCCACCTCCACCTGCCCCAC |
| | | TCACCACCTCTGCTAGTTCCAGACACCTCCCAAGCACGCAGCAATGCAGCTCAAAACGCTT |
| | | AGCCTAGCCACACCCCCACGGGAAACAGCAGTGATTAACCTTTAGCAATAAACGAAAGTTT |
| | | AACTAAGCTATACTAACCCCAGGGTTGGTCAATTTCGTGCCAGCCACACCCTGGAGCTAGC |
| | | AAAAAAAAAAAAAAAAAAAAAAAAAAAAAAGCATATGACTAAAAAAAAAAAAAAAAAAAAA |
| | | AAAAAAAAAAAAAAAAAAAAAAAAAAAAAAAAAAAAAAAAAAAAAAAAA |
| 37 | 5′UTR | GAATAAACTAGTATTCTTCTGGTCCCCACAGACTCAGAGAGAACCCGCCACCATGGGCCAG |
| | HBA1- | ACCAAGAGCAAGATCAAGTCTAAGTACGCCAGCTACCTGAGCTTCATCAAGATCCTGCTGA |
| | (partial) of | AGCGCGGAGGCGTGAAGGTGTCCACCAAGAACCTGATCAAGCTGTTCCAGATCATCGAGCA |
| | HERV-K | GTTCTGCCCCTGGTTTCCTGAGCAGGGCACCCTGGATCTGAAGGACTGGAAGCGGATCGGC |
| | Gag Env | AAAGAGCTGAAGCAGGCCGGCAGAAAGGGCAACATCATCCCTCTGACCGTGTGGAACGACT |
| | ISDWT- | GGGCCATCATTAAGGCCGCTCTGGAACCCTTCCAGACCGAAGAGGATAGCGTGTCCGTGTC |
| | AES | TGATGCCCCTGGCAGCTGCATCATCGACTGCAACGAGAACACCCGGAAGAAGTCCCAGAAA |
| | 3′UTR of | GAGACAGAGGGCCTGCACTGCGAGTATGTGGCCGAACCTGTGATGGCTCAGAGCACCCAGA |
| | mtRNR1- | ACGTGGACTACAACCAGCTGCAAGAAGTGATCTACCCCGAGACACTGAAGCTGGAAGGCAA |
| | linker-70) | GGGCCCTGAACTCGTGGGCCCTTCTGAGTCTAAGCCCAGAGGCACATCTCCTCTGCCTGCT |
| | PolyA (30- | GGACAGGTGCCAGTGACACTGCAGCCCCAGAAACAAGTGAAAGAGAACAAGACCCAGCCTC |
| | = “RNA8” | CTGTGGCCTACCAGTATTGGCCTCCAGCCGAGCTGCAGTACAGACCTCCTCCAGAGAGCCA |
| | (sequence | GTACGGCTACCCTGGAATGCCTCCTGCTCCTCAAGGCAGAGCCCCTTATCCTCAGCCTCCT |
| | encoding | ACCAGACGGCTGAACCCTACAGCTCCTCCTAGCAGACAGGGCTCTGAGCTGCACGAGATCA |
| | ISD in bold) | TTGACAAGAGCCGGAAAGAGGGCGACACCGAGGCTTGGCAGTTTCCCGTTACACTGGAACC |
| | | CATGCCTCCAGGCGAAGGCGCTCAAGAAGGCGAACCTCCTACAGTGGAAGCCCGGTACAAG |
| | | AGCTTCAGCATCAAAATGCTGAAGGACATGAAGGAAGGCGTCAAGCAGTACGGCCCTAACA |
| | | GCCCCTACATGCGGACCCTGCTGGATTCTATTGCCCACGGCCACCGGCTGATCCCTTACGA |
| | | TTGGGAGATCCTGGCCAAGTCCTCTCTGAGCCCTAGCCAGTTCCTGCAGTTCAAGACCTGG |
| | | TGGATCGACGGCGTGCAAGAACAAGTGCGGCGGAACAGAGCCGCCAATCCTCCTGTGAACA |
| | | TCGACGCCGATCAGCTGCTCGGAATCGGCCAGAATTGGAGCACCATCTCTCAGCAGGCCCT |
| | | GATGCAGAACGAGGCCATTGAACAAGTCCGGGCCATCTGCCTGAGAGCCTGGGAGAAGATT |
| | | CAGGACCCCGGCAGCACATGCCCCAGCTTTAATACCGTTCGGCAGGGCAGCAAAGAGCCCT |
| | | ATCCTGACTTTGTGGCCCGGCTGCAGGATGTGGCCCAGAAGTCTATTGCCGACGAGAAGGC |
| | | CCGGAAAGTGATCGTGGAACTGATGGCCTACGAGAACGCCAATCCAGAGTGCCAGAGCGCC |
| | | ATCAAGCCCCTGAAGGGAAAAGTGCCTGCCGGCTCCGATGTGATCAGCGAGTACGTGAAGG |
| | | CCTGCGACGGAATTGGCGGAGCCATGCACAAAGCCATGCTGATGGCACAGGCCATCACTGG |
| | | CGTTGTGCTCGGAGGACAAGTTCGGACCTTTGGCGGCAAGTGCTACAACTGTGGCCAGATC |
| | | GGACACCTGAAGAAAAACTGCCCCGTGCTGAACAAGCAGAACATCACCATCCAGGCCACCA |
| | | CCACCGGCAGAGAACCTCCAGATCTGTGCCCTAGATGCAAGAAGGGCAAGCACTGGGCCAG |
| | | CCAGTGCAGAAGCAAGTTCGACAAGAACGGCCAGCCTCTGAGCGGCAACGAGCAAAGAGGA |
| | | CAGCCTCAGGCTCCTCAGCAGACCGGCGCATTTCCAATCCAGCCTTTCGTGCCCCAAGGCT |
| | | TCCAGGGACAACAGCCTCCACTGAGCCAAGTGTTCCAGGGCATTAGCCAGCTGCCTCAGTA |
| | | CAACAACTGCCCTCCACCTCAGGCTGCCGTGCAGCAGGGATCAGGCGCTACAAATTTTAGC |
| | | CTGCTGAAGCAGGCAGGGGATGTGGAGGAAAACCCCGGACCTATGAACCCCAGCGAGATGC |
| | | AGAGAAAGGCCCCACCTAGACGGCGGAGACACAGAAATAGAGCCCCTCTGACACACAAGAT |
| | | GAACAAGATGGTCACCAGCGAGGAACAGATGAAGCTGCCCAGCACCAAGAAGGCCGAGCCT |
| | | CCAACATGGGCCCAGCTGAAGAAACTGACCCAGCTGGCCACCAAGTACCTGGAAAACACCA |
| | | AAGTGACCCAGACACCTGAGAGCATGCTGCTGGCCGCTCTGATGATCGTGTCCATGGTGGT |
| | | GTCCCTGCCTATGCCTGCTGGTGCCGCCGCTGCCAATTACACATACTGGGCCTACGTGCCC |
| | | TTTCCTCCTCTGATTCGGGCCGTGACCTGGATGGACAACCCCATCGAAGTGTACGTGAACG |
| | | ACAGCGTGTGGGTGCCCGGACCAATCGACGATAGATGTCCCGCCAAGCCTGAGGAAGAGGG |
| | | CATGATGATCAACATCAGCATCGGCTACCGGTATCCTCCAATCTGCCTGGGCAGAGCCCCT |
| | | GGCTGTCTTATGCCAGCTGTGCAGAACTGGCTGGTGGAAGTGCCTACCGTGTCTCCCATCA |
| | | GCCGGTTCACCTACCACATGGTGTCCGGCATGAGCCTGCGGCCTAGAGTGAACTACCTGCA |
| | | GGACTTCAGCTACCAGCGGAGCCTGAAGTTCAGACCCAAGGGCAAGCCCTGTCCTAAAGAG |
| | | ATCCCCAAAGAGTCCAAGAACACCGAGGTGCTCGTGTGGGAAGAGTGCGTGGCCAATTCTG |
| | | CCGTGATCCTGCAGAACAACGAGTTCGGCACCATCATCGACTGGGCCCCTAGAGGCCAGTT |
| | | CTACCACAATTGCAGCGGCCAGACACAGAGCTGCCCTAGCGCTCAAGTGTCACCAGCCGTG |
| | | GATAGCGATCTGACCGAGAGCCTGGACAAGCACAAACACAAGAAGCTGCAGAGCTTCTACC |
| | | CCTGGGAGTGGGGCGAGAAGGGCATCTCTACACCCAGACCTAAGATCGTGTCACCCGTGTC |
| | | CGGACCTGAGCACCCTGAACTTTGGAGACTGACAGTGGCCAGCCACCACATCAGAATTTGG |
| | | AGCGGCAATCAGACCCTGGAAACCCGGGACAGAAAGCCCTTCTACACCGTGGACCTGAACA |
| | | GCAGCCTGACCGTGCCTCTCCAGAGCTGTGTGAAGCCTCCTTACATGCTGGTCGTGGGCAA |
| | | CATCGTGATCAAGCCCGACAGCCAGACCATCACATGCGAGAACTGCAGACTGCTGACCTGC |
| | | ATCGACAGCACCTTCAACTGGCAGCACCGGATCCTGCTCGTGCGAGCTAGAGAAGGCGTGT |
| | | GGATCCCCGTGTCTATGGACAGACCTTGGGAAGCTAGCCCCAGCGTGCACATCCTGACAGA |
| | | GGTGCTGAAGGGCGTGCTGAACAGAAGCAAGCGGTTCATCTTCACCCTGATCGCCGTGATC |
| | | ATGGGCCTGATTGCTGTGACAGCCACAGCTGCTGTTGCTGGCGTGGCCCTGCATAGCTCTG |
| | | TGCAGAGCGTGAACTTCGTGAACGATTGGCAGAAGAACAGCACCCGGCTGTGGAACAGCCA |
| | | GAGCAGCATCGATCAGAAGCTGGCCAACCAGATCAACGACCTGCGGCAGACAGTGATCTGG |
| | | ATGGGCGACAGACTGATGAGCCTGGAACACCGGTTCCAGCTGCAGTGCGACTGGAATACCA |
| | | GCGACTTCTGCATCACCCCTCAGATCTACAACGAGAGCGAGCACCACTGGGATATGGTCCG |
| | | AAGGCATCTGCAGGGCAGAGAGGACAACCTGACACTGGACATCAGCAAGCTGAAAGAGCAG |
| | | ATCTTCGAGGCCAGCAAGGCCCACCTGAATCTGGTGCCTGGAACCGAAGCTATTGCCGGCG |
| | | TTGCAGATGGCCTGGCCAATCTGAATCCTGTGACCTGGGTCAAGACCATCGGCAGCACCAC |
| | | AATCATCAACCTGATCCTGATCCTCGTGTGCCTGTTCTGCCTGCTGCTTGTGTGCAGATGC |
| | | ACCCAGCAGCTGAGAAGAGACAGCGACCATAGAGAACGGGCCATGATGACCATGGCCGTGC |
| | | TGAGCAAGAGAAAAGGCGGCAACGTGGGCAAGAGCAAGCGGGATCAGATCGTGACCGTGTC |
| | | CGTGTGATGACTCGAGCTGGTACTGCATGCACGCAATGCTAGCTGCCCCTTTCCCGTCCTG |
| | | GGTACCCCGAGTCTCCCCCGACCTCGGGTCCCAGGTATGCTCCCACCTCCACCTGCCCCAC |
| | | TCACCACCTCTGCTAGTTCCAGACACCTCCCAAGCACGCAGCAATGCAGCTCAAAACGCTT |
| | | AGCCTAGCCACACCCCCACGGGAAACAGCAGTGATTAACCTTTAGCAATAAACGAAAGTTT |
| | | AACTAAGCTATACTAACCCCAGGGTTGGTCAATTTCGTGCCAGCCACACCCTGGAGCTAGC |
| | | AAAAAAAAAAAAAAAAAAAAAAAAAAAAAAGCATATGACTAAAAAAAAAAAAAAAAAAAAA |
| | | AAAAAAAAAAAAAAAAAAAAAAAAAAAAAAAAAAAAAAAAAAAAAAAAA |

In any of the sequences according to SEQ ID NO:s 5, 45, 36 and 37 T may be replaced with m1Ψ (N1-methylpseudouridine). This may have an mRNA stabilizing effect.

The sequence of SEQ ID NO:s 5, may comprise on DNA level the initial nucleotide sequence GGG preceding the 5′UTR.

The sequences of SEQ ID NO: 36 and 37 may comprise on DNA level the initial nucleotide sequence GGGA preceding the 5′ UTR.

The sequence of SEQ ID NO: 45 may comprise on DNA level the initial nucleotide sequence GGGAG preceding the 5′ UTR.

1. A composition comprising a transfection agent and an mRNA which encodes at least a human endogenous retrovirus (HERV) envelope protein or an immunogenic part thereof; wherein said HERV envelope protein comprises an immune-suppressive domain (ISD) wherein said human endogenous retrovirus (HERV) is HERV-K and wherein the HERV Env protein comprises a mutated immune-suppressive domain (ISD), wherein the ISD mutation is selected from Q525A, L522A, A523Q, N524A, N527A, L529A, R530A, Q531A and T532A;
and/or
wherein the HERV envelope protein comprises amino acid mutation S190C.

2. The composition of claim 1, wherein the composition comprises lipid nanoparticles (LNPs) that comprise said mRNA.

3. The composition of claim 1, wherein the transfection agent is a transfection agent that comprises a cationic lipid and/or a cationic polymer.

4-7. (canceled)

8. The composition of claim 1, wherein the mRNA comprises at least 300 nucleotides.

9. The composition of claim 1, wherein the mRNA comprises no artificially modified nucleotides.

10. (canceled)

11. The composition of claim 1, wherein the HERV envelope protein comprises an amino acid sequence having at least 95% identity to the amino acid sequence of SEQ ID NO: 2 over the entire length of SEQ ID NO: 2.

12. (canceled)

13. The composition of claim 1, wherein the mutated ISD comprises the amino acid sequence:

| L | A | N | Q | I | N | D | L | R | Q | T | V | I | W |
|---|---|---|---|---|---|---|---|---|---|---|---|---|---|
| 1 | 2 | 3 | 4 | 5 | 6 | 7 | 8 | 9 | 10 | 11 | 12 | 13 | 14 |

wherein one or two single amino acids at any of the positions 1, 2, 3, 4, 5, 6, 7, 8, 9, 10, 11, 12, 13 and 14 are replaced with alanine in each instance, to render the ISD inactive.

14. (canceled)

15. The composition of claim 1, wherein the mutated ISD comprises amino acid sequence LANAINDLRQTVIW (SEQ ID NO: 4).

16. The composition of claim 1,
wherein the HERV envelope protein comprises the sequence VQNWLVEVPTVSPICRFTYHMVSGMSLRP; and/or wherein the HERV envelope protein comprises an amino acid sequence having at least 90% sequence identity with SEQ ID NO: 2, wherein the HERV envelope protein comprises a cysteine at a position within said HERV envelope protein that corresponds to amino acid 190 of SEQ ID NO: 2.

17. The composition of claim 1, wherein the HERV envelope protein comprises a sequence selected from the group consisting of SEQ ID NO:s 21-35.

18. The composition of claim 1, wherein the HERV envelope protein comprises a sequence selected from the group consisting of SEQ ID NOs: 22-27, 29-32 and 35.

19. The composition of claim 1, wherein the composition comprises an mRNA which has at least 90% sequence identity with any of the sequences according to any one of SEQ ID NO:s 45, 36 or 37.

20. The composition according to claim 16, wherein cell surface expression of the HERV envelope protein is increased compared to the cell surface expression of a HERV envelope protein having an amino acid sequence according to SEQ ID: 2.

21-24. (canceled)

25. A method for treatment or prevention of a disease in a patient in need thereof, said method comprising:
administering to the patient a pharmaceutically acceptable liquid composition comprising a therapeutically effective amount of the composition of claim 1.

26. The method of claim 25, wherein said disease is selected from the group consisting of cancer, HIV and/or associated disorders, rheumatic diseases, neurodegenerative diseases, aging associated diseases, diseases associated with HERV reactivation, chronic inflammation multiple sclerosis, ALS, sarcopenia, kidney diseases and Alzheimer's disease.

27. The method of claim 26,
wherein the cancer is an HERV-expressing cancer.

28. (canceled)

29. (canceled)

30. A pharmaceutical composition comprising the composition of claim 1 and a pharmaceutically acceptable excipient.

31. A DNA molecule encoding an mRNA which encodes at least a human endogenous retrovirus (HERV) envelope protein or an immunogenic part thereof; wherein said HERV envelope protein comprises an immune-suppressive domain (ISD), wherein said human endogenous retrovirus (HERV) is HERV-K and wherein the HERV Env protein comprises a mutated immune-suppressive domain (ISD), wherein the ISD mutation is selected from Q525A, L522A, A523Q, N524A, N527A, L529A, R530A, Q531A and T532A;
and/or
wherein the HERV envelope protein comprises amino acid mutation S190C.

32. A virus like particle (VLP) comprising a HERV envelope protein, wherein said HERV envelope protein comprises an immune-suppressive domain (ISD), wherein said human endogenous retrovirus (HERV) is HERV-K and wherein the HERV Env protein comprises a mutated immune-suppressive domain (ISD), wherein the ISD mutation is selected from Q525A, L522A, A523Q, N524A, N527A, L529A, R530A, Q531A and T532A;
and/or
wherein the HERV envelope protein comprises amino acid mutation S190C.


SEQUENCE LISTING (51 sequences)

SEQ ID NO: 1 - protein, 699 aa, Homo sapiens
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPIEVYVNDSVWVPGPIDDRCPAKPEEEGMMINISIGYRYPPICLGRAPGCLMPAVQNWLVEVPTVSPISRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIVSPVSGPEHPELWRLTVASHHIRIWSGNQTLETRDRKPFYTVDLNSSLTVPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDWQKNSTRLWNSQSSIDQKLANQINDLRQTVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 2 - protein, 699 aa, synthetic construct
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPIEVYVNDSVWVPGPIDDRCPAKPEEEGMMINISIGYRYPPICLGRAPGCLMPAVQNWLVEVPTVSPISRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIVSPVSGPEHPELWRLTVASHHIRIWSGNQTLETRDRKPFYTVDLNSSLTVPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDWQKNSTRLWNSQSSIDQKLANAINDLRQTVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 3 - protein, 14 aa, Homo sapiens
LANQINDLRQTVIW

SEQ ID NO: 4 - protein, 14 aa, synthetic construct
LANAINDLRQTVIW

SEQ ID NO: 5 - RNA, 4408 nt, synthetic construct
  source 1..4408: mol_type = other RNA
agactcttctggtccccacagactcagagagaagccaccatgggccagaccaagagcaagatcaagtctaagtacgccagctacctgagcttcatcaagatcctgctgaagcgcggaggcgtgaaggtgtccaccaagaacctgatcaagctgttccagatcatcgagcagttctgcccctggtttcctgagcagggcaccctggatctgaaggactggaagcggatcggcaaagagctgaagcaggccggcagaaagggcaacatcatccctctgaccgtgtggaacgactgggccatcattaaggccgctctggaacccttccagaccgaagaggatagcgtgtccgtgtctgatgcccctggcagctgcatcatcgactgcaacgagaacacccggaagaagtcccagaaagagacagagggcctgcactgcgagtatgtggccgaacctgtgatggctcagagcacccagaacgtggactacaaccagctgcaagaagtgatctaccccgagacactgaagctggaaggcaagggccctgaactcgtgggcccttctgagtctaagcccagaggcacatctcctctgcctgctggacaggtgccagtgacactgcagccccagaaacaagtgaaagagaacaagacccagcctcctgtggcctaccagtattggcctccagccgagctgcagtacagacctcctccagagagccagtacggctaccctggaatgcctcctgctcctcaaggcagagccccttatcctcagcctcctaccagacggctgaaccctacagctcctcctagcagacagggctctgagctgcacgagatcattgacaagagccggaaagagggcgacaccgaggcttggcagtttcccgttacactggaacccatgcctccaggcgaaggcgctcaagaaggcgaacctcctacagtggaagcccggtacaagagcttcagcatcaaaatgctgaaggacatgaaggaaggcgtcaagcagtacggccctaacagcccctacatgcggaccctgctggattctattgcccacggccaccggctgatcccttacgattgggagatcctggccaagtcctctctgagccctagccagttcctgcagttcaagacctggtggatcgacggcgtgcaagaacaagtgcggcggaacagagccgccaatcctcctgtgaacatcgacgccgatcagctgctcggaatcggccagaattggagcaccatctctcagcaggccctgatgcagaacgaggccattgaacaagtccgggccatctgcctgagagcctgggagaagattcaggaccccggcagcacatgccccagctttaataccgttcggcagggcagcaaagagccctatcctgactttgtggcccggctgcaggatgtggcccagaagtctattgccgacgagaaggcccggaaagtgatcgtggaactgatggcctacgagaacgccaatccagagtgccagagcgccatcaagcccctgaagggaaaagtgcctgccggctccgatgtgatcagcgagtacgtgaaggcctgcgacggaattggcggagccatgcacaaagccatgctgatggcacaggccatcactggcgttgtgctcggaggacaagttcggacctttggcggcaagtgctacaactgtggccagatcggacacctgaagaaaaactgccccgtgctgaacaagcagaacatcaccatccaggccaccaccaccggcagagaacctccagatctgtgccctagatgcaagaagggcaagcactgggccagccagtgcagaagcaagttcgacaagaacggccagcctctgagcggcaacgagcaaagaggacagcctcaggctcctcagcagaccggcgcatttccaatccagcctttcgtgccccaaggcttccagggacaacagcctccactgagccaagtgttccagggcattagccagctgcctcagtacaacaactgccctccacctcaggctgccgtgcagcagggatcaggcgctacaaattttagcctgctgaagcaggcaggggatgtggaggaaaaccccggacctatgaaccccagcgagatgcagagaaaggccccacctagacggcggagacacagaaatagagcccctctgacacacaagatgaacaagatggtcaccagcgaggaacagatgaagctgcccagcaccaagaaggccgagcctccaacatgggcccagctgaagaaactgacccagctggccaccaagtacctggaaaacaccaaagtgacccagacacctgagagcatgctgctggccgctctgatgatcgtgtccatggtggtgtccctgcctatgcctgctggtgccgccgctgccaattacacatactgggcctacgtgccctttcctcctctgattcgggccgtgacctggatggacaaccccatcgaagtgtacgtgaacgacagcgtgtgggtgcccggaccaatcgacgatagatgtcccgccaagcctgaggaagagggcatgatgatcaacatcagcatcggctaccggtatcctccaatctgcctgggcagagcccctggctgtcttatgccagctgtgcagaactggctggtggaagtgcctaccgtgtctcccatcagccggttcacctaccacatggtgtccggcatgagcctgcggcctagagtgaactacctgcaggacttcagctaccagcggagcctgaagttcagacccaagggcaagccctgtcctaaagagatccccaaagagtccaagaacaccgaggtgctcgtgtgggaagagtgcgtggccaattctgccgtgatcctgcagaacaacgagttcggcaccatcatcgactgggcccctagaggccagttctaccacaattgcagcggccagacacagagctgccctagcgctcaagtgtcaccagccgtggatagcgatctgaccgagagcctggacaagcacaaacacaagaagctgcagagcttctacccctgggagtggggcgagaagggcatctctacacccagacctaagatcgtgtcacccgtgtccggacctgagcaccctgaactttggagactgacagtggccagccaccacatcagaatttggagcggcaatcagaccctggaaacccgggacagaaagcccttctacaccgtggacctgaacagcagcctgaccgtgcctctccagagctgtgtgaagcctccttacatgctggtcgtgggcaacatcgtgatcaagcccgacagccagaccatcacatgcgagaactgcagactgctgacctgcatcgacagcaccttcaactggcagcaccggatcctgctcgtgcgagctagagaaggcgtgtggatccccgtgtctatggacagaccttgggaagctagccccagcgtgcacatcctgacagaggtgctgaagggcgtgctgaacagaagcaagcggttcatcttcaccctgatcgccgtgatcatgggcctgattgctgtgacagccacagctgctgttgctggcgtggccctgcatagctctgtgcagagcgtgaacttcgtgaacgattggcagaagaacagcacccggctgtggaacagccagagcagcatcgatcagaagctggccaacgccatcaacgacctgcggcagacagtgatctggatgggcgacagactgatgagcctggaacaccggttccagctgcagtgcgactggaataccagcgacttctgcatcacccctcagatctacaacgagagcgagcaccactgggatatggtccgaaggcatctgcagggcagagaggacaacctgacactggacatcagcaagctgaaagagcagatcttcgaggccagcaaggcccacctgaatctggtgcctggaaccgaagctattgccggcgttgcagatggcctggccaatctgaatcctgtgacctgggtcaagaccatcggcagcaccacaatcatcaacctgatcctgatcctcgtgtgcctgttctgcctgctgcttgtgtgcagatgcacccagcagctgagaagagacagcgaccatagagaacgggccatgatgaccatggccgtgctgagcaagagaaaaggcggcaacgtgggcaagagcaagcgggatcagatcgtgaccgtgtccgtgtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaa

SEQ ID NO: 6 - RNA, 4164 nt, synthetic construct
  source 1..4164: mol_type = other RNA
atgggccagaccaagagcaagatcaagtctaagtacgccagctacctgagcttcatcaagatcctgctgaagcgcggaggcgtgaaggtgtccaccaagaacctgatcaagctgttccagatcatcgagcagttctgcccctggtttcctgagcagggcaccctggatctgaaggactggaagcggatcggcaaagagctgaagcaggccggcagaaagggcaacatcatccctctgaccgtgtggaacgactgggccatcattaaggccgctctggaacccttccagaccgaagaggatagcgtgtccgtgtctgatgcccctggcagctgcatcatcgactgcaacgagaacacccggaagaagtcccagaaagagacagagggcctgcactgcgagtatgtggccgaacctgtgatggctcagagcacccagaacgtggactacaaccagctgcaagaagtgatctaccccgagacactgaagctggaaggcaagggccctgaactcgtgggcccttctgagtctaagcccagaggcacatctcctctgcctgctggacaggtgccagtgacactgcagccccagaaacaagtgaaagagaacaagacccagcctcctgtggcctaccagtattggcctccagccgagctgcagtacagacctcctccagagagccagtacggctaccctggaatgcctcctgctcctcaaggcagagccccttatcctcagcctcctaccagacggctgaaccctacagctcctcctagcagacagggctctgagctgcacgagatcattgacaagagccggaaagagggcgacaccgaggcttggcagtttcccgttacactggaacccatgcctccaggcgaaggcgctcaagaaggcgaacctcctacagtggaagcccggtacaagagcttcagcatcaaaatgctgaaggacatgaaggaaggcgtcaagcagtacggccctaacagcccctacatgcggaccctgctggattctattgcccacggccaccggctgatcccttacgattgggagatcctggccaagtcctctctgagccctagccagttcctgcagttcaagacctggtggatcgacggcgtgcaagaacaagtgcggcggaacagagccgccaatcctcctgtgaacatcgacgccgatcagctgctcggaatcggccagaattggagcaccatctctcagcaggccctgatgcagaacgaggccattgaacaagtccgggccatctgcctgagagcctgggagaagattcaggaccccggcagcacatgccccagctttaataccgttcggcagggcagcaaagagccctatcctgactttgtggcccggctgcaggatgtggcccagaagtctattgccgacgagaaggcccggaaagtgatcgtggaactgatggcctacgagaacgccaatccagagtgccagagcgccatcaagcccctgaagggaaaagtgcctgccggctccgatgtgatcagcgagtacgtgaaggcctgcgacggaattggcggagccatgcacaaagccatgctgatggcacaggccatcactggcgttgtgctcggaggacaagttcggacctttggcggcaagtgctacaactgtggccagatcggacacctgaagaaaaactgccccgtgctgaacaagcagaacatcaccatccaggccaccaccaccggcagagaacctccagatctgtgccctagatgcaagaagggcaagcactgggccagccagtgcagaagcaagttcgacaagaacggccagcctctgagcggcaacgagcaaagaggacagcctcaggctcctcagcagaccggcgcatttccaatccagcctttcgtgccccaaggcttccagggacaacagcctccactgagccaagtgttccagggcattagccagctgcctcagtacaacaactgccctccacctcaggctgccgtgcagcagggatcaggcgctacaaattttagcctgctgaagcaggcaggggatgtggaggaaaaccccggacctatgaaccccagcgagatgcagagaaaggccccacctagacggcggagacacagaaatagagcccctctgacacacaagatgaacaagatggtcaccagcgaggaacagatgaagctgcccagcaccaagaaggccgagcctccaacatgggcccagctgaagaaactgacccagctggccaccaagtacctggaaaacaccaaagtgacccagacacctgagagcatgctgctggccgctctgatgatcgtgtccatggtggtgtccctgcctatgcctgctggtgccgccgctgccaattacacatactgggcctacgtgccctttcctcctctgattcgggccgtgacctggatggacaaccccatcgaagtgtacgtgaacgacagcgtgtgggtgcccggaccaatcgacgatagatgtcccgccaagcctgaggaagagggcatgatgatcaacatcagcatcggctaccggtatcctccaatctgcctgggcagagcccctggctgtcttatgccagctgtgcagaactggctggtggaagtgcctaccgtgtctcccatcagccggttcacctaccacatggtgtccggcatgagcctgcggcctagagtgaactacctgcaggacttcagctaccagcggagcctgaagttcagacccaagggcaagccctgtcctaaagagatccccaaagagtccaagaacaccgaggtgctcgtgtgggaagagtgcgtggccaattctgccgtgatcctgcagaacaacgagttcggcaccatcatcgactgggcccctagaggccagttctaccacaattgcagcggccagacacagagctgccctagcgctcaagtgtcaccagccgtggatagcgatctgaccgagagcctggacaagcacaaacacaagaagctgcagagcttctacccctgggagtggggcgagaagggcatctctacacccagacctaagatcgtgtcacccgtgtccggacctgagcaccctgaactttggagactgacagtggccagccaccacatcagaatttggagcggcaatcagaccctggaaacccgggacagaaagcccttctacaccgtggacctgaacagcagcctgaccgtgcctctccagagctgtgtgaagcctccttacatgctggtcgtgggcaacatcgtgatcaagcccgacagccagaccatcacatgcgagaactgcagactgctgacctgcatcgacagcaccttcaactggcagcaccggatcctgctcgtgcgagctagagaaggcgtgtggatccccgtgtctatggacagaccttgggaagctagccccagcgtgcacatcctgacagaggtgctgaagggcgtgctgaacagaagcaagcggttcatcttcaccctgatcgccgtgatcatgggcctgattgctgtgacagccacagctgctgttgctggcgtggccctgcatagctctgtgcagagcgtgaacttcgtgaacgattggcagaagaacagcacccggctgtggaacagccagagcagcatcgatcagaagctggccaacgccatcaacgacctgcggcagacagtgatctggatgggcgacagactgatgagcctggaacaccggttccagctgcagtgcgactggaataccagcgacttctgcatcacccctcagatctacaacgagagcgagcaccactgggatatggtccgaaggcatctgcagggcagagaggacaacctgacactggacatcagcaagctgaaagagcagatcttcgaggccagcaaggcccacctgaatctggtgcctggaaccgaagctattgccggcgttgcagatggcctggccaatctgaatcctgtgacctgggtcaagaccatcggcagcaccacaatcatcaacctgatcctgatcctcgtgtgcctgttctgcctgctgcttgtgtgcagatgcacccagcagctgagaagagacagcgaccatagagaacgggccatgatgaccatggccgtgctgagcaagagaaaaggcggcaacgtgggcaagagcaagcgggatcagatcgtgaccgtgtccgtgtga

SEQ ID NO: 7 - RNA, 4164 nt, synthetic construct
  source 1..4164: mol_type = other RNA
atgggccagaccaagagcaagatcaagtctaagtacgccagctacctgagcttcatcaagatcctgctgaagcgcggaggcgtgaaggtgtccaccaagaacctgatcaagctgttccagatcatcgagcagttctgcccctggtttcctgagcagggcaccctggatctgaaggactggaagcggatcggcaaagagctgaagcaggccggcagaaagggcaacatcatccctctgaccgtgtggaacgactgggccatcattaaggccgctctggaacccttccagaccgaagaggatagcgtgtccgtgtctgatgcccctggcagctgcatcatcgactgcaacgagaacacccggaagaagtcccagaaagagacagagggcctgcactgcgagtatgtggccgaacctgtgatggctcagagcacccagaacgtggactacaaccagctgcaagaagtgatctaccccgagacactgaagctggaaggcaagggccctgaactcgtgggcccttctgagtctaagcccagaggcacatctcctctgcctgctggacaggtgccagtgacactgcagccccagaaacaagtgaaagagaacaagacccagcctcctgtggcctaccagtattggcctccagccgagctgcagtacagacctcctccagagagccagtacggctaccctggaatgcctcctgctcctcaaggcagagccccttatcctcagcctcctaccagacggctgaaccctacagctcctcctagcagacagggctctgagctgcacgagatcattgacaagagccggaaagagggcgacaccgaggcttggcagtttcccgttacactggaacccatgcctccaggcgaaggcgctcaagaaggcgaacctcctacagtggaagcccggtacaagagcttcagcatcaaaatgctgaaggacatgaaggaaggcgtcaagcagtacggccctaacagcccctacatgcggaccctgctggattctattgcccacggccaccggctgatcccttacgattgggagatcctggccaagtcctctctgagccctagccagttcctgcagttcaagacctggtggatcgacggcgtgcaagaacaagtgcggcggaacagagccgccaatcctcctgtgaacatcgacgccgatcagctgctcggaatcggccagaattggagcaccatctctcagcaggccctgatgcagaacgaggccattgaacaagtccgggccatctgcctgagagcctgggagaagattcaggaccccggcagcacatgccccagctttaataccgttcggcagggcagcaaagagccctatcctgactttgtggcccggctgcaggatgtggcccagaagtctattgccgacgagaaggcccggaaagtgatcgtggaactgatggcctacgagaacgccaatccagagtgccagagcgccatcaagcccctgaagggaaaagtgcctgccggctccgatgtgatcagcgagtacgtgaaggcctgcgacggaattggcggagccatgcacaaagccatgctgatggcacaggccatcactggcgttgtgctcggaggacaagttcggacctttggcggcaagtgctacaactgtggccagatcggacacctgaagaaaaactgccccgtgctgaacaagcagaacatcaccatccaggccaccaccaccggcagagaacctccagatctgtgccctagatgcaagaagggcaagcactgggccagccagtgcagaagcaagttcgacaagaacggccagcctctgagcggcaacgagcaaagaggacagcctcaggctcctcagcagaccggcgcatttccaatccagcctttcgtgccccaaggcttccagggacaacagcctccactgagccaagtgttccagggcattagccagctgcctcagtacaacaactgccctccacctcaggctgccgtgcagcagggatcaggcgctacaaattttagcctgctgaagcaggcaggggatgtggaggaaaaccccggacctatgaaccccagcgagatgcagagaaaggccccacctagacggcggagacacagaaatagagcccctctgacacacaagatgaacaagatggtcaccagcgaggaacagatgaagctgcccagcaccaagaaggccgagcctccaacatgggcccagctgaagaaactgacccagctggccaccaagtacctggaaaacaccaaagtgacccagacacctgagagcatgctgctggccgctctgatgatcgtgtccatggtggtgtccctgcctatgcctgctggtgccgccgctgccaattacacatactgggcctacgtgccctttcctcctctgattcgggccgtgacctggatggacaaccccatcgaagtgtacgtgaacgacagcgtgtgggtgcccggaccaatcgacgatagatgtcccgccaagcctgaggaagagggcatgatgatcaacatcagcatcggctaccggtatcctccaatctgcctgggcagagcccctggctgtcttatgccagctgtgcagaactggctggtggaagtgcctaccgtgtctcccatcagccggttcacctaccacatggtgtccggcatgagcctgcggcctagagtgaactacctgcaggacttcagctaccagcggagcctgaagttcagacccaagggcaagccctgtcctaaagagatccccaaagagtccaagaacaccgaggtgctcgtgtgggaagagtgcgtggccaattctgccgtgatcctgcagaacaacgagttcggcaccatcatcgactgggcccctagaggccagttctaccacaattgcagcggccagacacagagctgccctagcgctcaagtgtcaccagccgtggatagcgatctgaccgagagcctggacaagcacaaacacaagaagctgcagagcttctacccctgggagtggggcgagaagggcatctctacacccagacctaagatcgtgtcacccgtgtccggacctgagcaccctgaactttggagactgacagtggccagccaccacatcagaatttggagcggcaatcagaccctggaaacccgggacagaaagcccttctacaccgtggacctgaacagcagcctgaccgtgcctctccagagctgtgtgaagcctccttacatgctggtcgtgggcaacatcgtgatcaagcccgacagccagaccatcacatgcgagaactgcagactgctgacctgcatcgacagcaccttcaactggcagcaccggatcctgctcgtgcgagctagagaaggcgtgtggatccccgtgtctatggacagaccttgggaagctagccccagcgtgcacatcctgacagaggtgctgaagggcgtgctgaacagaagcaagcggttcatcttcaccctgatcgccgtgatcatgggcctgattgctgtgacagccacagctgctgttgctggcgtggccctgcatagctctgtgcagagcgtgaacttcgtgaacgattggcagaagaacagcacccggctgtggaacagccagagcagcatcgatcagaagctggccaaccagatcaacgacctgcggcagacagtgatctggatgggcgacagactgatgagcctggaacaccggttccagctgcagtgcgactggaataccagcgacttctgcatcacccctcagatctacaacgagagcgagcaccactgggatatggtccgaaggcatctgcagggcagagaggacaacctgacactggacatcagcaagctgaaagagcagatcttcgaggccagcaaggcccacctgaatctggtgcctggaaccgaagctattgccggcgttgcagatggcctggccaatctgaatcctgtgacctgggtcaagaccatcggcagcaccacaatcatcaacctgatcctgatcctcgtgtgcctgttctgcctgctgcttgtgtgcagatgcacccagcagctgagaagagacagcgaccatagagaacgggccatgatgaccatggccgtgctgagcaagagaaaaggcggcaacgtgggcaagagcaagcgggatcagatcgtgaccgtgtccgtgtga

SEQ ID NO: 8 - protein, 14 aa, synthetic construct
AANQINDLRQTVIW

SEQ ID NO: 9 - protein, 14 aa, synthetic construct
LQNQINDLRQTVIW

SEQ ID NO: 10 - protein, 14 aa, synthetic construct
LAAQINDLRQTVIW

SEQ ID NO: 11 - protein, 14 aa, synthetic construct
LANQANDLRQTVIW

SEQ ID NO: 12 - protein, 14 aa, synthetic construct
LANQIADLRQTVIW

SEQ ID NO: 13 - protein, 14 aa, synthetic construct
LANQINALRQTVIW

SEQ ID NO: 14 - protein, 14 aa, synthetic construct
LANQINDARQTVIW

SEQ ID NO: 15 - protein, 14 aa, synthetic construct
LANQINDLAQTVIW

SEQ ID NO: 16 - protein, 14 aa, synthetic construct
LANQINDLRATVIW

SEQ ID NO: 17 - protein, 14 aa, synthetic construct
LANQINDLRQAVIW

SEQ ID NO: 18 - protein, 14 aa, synthetic construct
LANQINDLRQTAIW

SEQ ID NO: 19 - protein, 14 aa, synthetic construct
LANQINDLRQTVAW

SEQ ID NO: 20 - protein, 14 aa, synthetic construct
LANQINDLRQTVIA

SEQ ID NO: 21 - protein, 699 aa, synthetic construct
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPIEVYVNDSVWVPGPIDDRCPAKPEEEGMMINISIGYRYPPICLGRAPGCLMPAVQNWLVEVPTVSPICRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIVSPVSGPEHPELWRLTVASHHIRIWSGNQTLETRDRKPFYTVDLNSSLTVPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDWQKNSTRLWNSQSSIDQKLANQINDLRQTVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 22 - protein, 699 aa, synthetic construct
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPIEVYVNDSVWVPGPIDDRCPAKPEEEGMMINISIGYRYPPICLGRAPGCLMPAVQNWLVEVPTVSPICRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIVSPVSGPEHPELWRLTVASHHIRIWSGNQTLETRDRKPFYTVDLNSSLTVPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDWQKNSTRLWNSQSSIDQKAANQINDLRQTVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 23 - protein, 699 aa, synthetic construct
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPIEVYVNDSVWVPGPIDDRCPAKPEEEGMMINISIGYRYPPICLGRAPGCLMPAVQNWLVEVPTVSPICRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIVSPVSGPEHPELWRLTVASHHIRIWSGNQTLETRDRKPFYTVDLNSSLTVPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDWQKNSTRLWNSQSSIDQKLQNQINDLRQTVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 24 - protein, 699 aa, synthetic construct
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPIEVYVNDSVWVPGPIDDRCPAKPEEEGMMINISIGYRYPPICLGRAPGCLMPAVQNWLVEVPTVSPICRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIVSPVSGPEHPELWRLTVASHHIRIWSGNQTLETRDRKPFYTVDLNSSLTVPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDWQKNSTRLWNSQSSIDQKLAAQINDLRQTVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 25 - protein, 699 aa, synthetic construct
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPIEVYVNDSVWVPGPIDDRCPAKPEEEGMMINISIGYRYPPICLGRAPGCLMPAVQNWLVEVPTVSPICRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIVSPVSGPEHPELWRLTVASHHIRIWSGNQTLETRDRKPFYTVDLNSSLTVPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDWQKNSTRLWNSQSSIDQKLANAINDLRQTVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 26 - protein, 699 aa, synthetic construct
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPIEVYVNDSVWVPGPIDDRCPAKPEEEGMMINISIGYRYPPICLGRAPGCLMPAVQNWLVEVPTVSPICRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIVSPVSGPEHPELWRLTVASHHIRIWSGNQTLETRDRKPFYTVDLNSSLTVPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDWQKNSTRLWNSQSSIDQKLANQANDLRQTVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 27 - protein, 699 aa, synthetic construct
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPIEVYVNDSVWVPGPIDDRCPAKPEEEGMMINISIGYRYPPICLGRAPGCLMPAVQNWLVEVPTVSPICRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIVSPVSGPEHPELWRLTVASHHIRIWSGNQTLETRDRKPFYTVDLNSSLTVPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDWQKNSTRLWNSQSSIDQKLANQIADLRQTVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 28 - protein, 699 aa, synthetic construct
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPIEVYVNDSVWVPGPIDDRCPAKPEEEGMMINISIGYRYPPICLGRAPGCLMPAVQNWLVEVPTVSPICRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIVSPVSGPEHPELWRLTVASHHIRIWSGNQTLETRDRKPFYTVDLNSSLTVPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDWQKNSTRLWNSQSSIDQKLANQINALRQTVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 29 - protein, 699 aa, synthetic construct
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPIEVYVNDSVWVPGPIDDRCPAKPEEEGMMINISIGYRYPPICLGRAPGCLMPAVQNWLVEVPTVSPICRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIVSPVSGPEHPELWRLTVASHHIRIWSGNQTLETRDRKPFYTVDLNSSLTVPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDWQKNSTRLWNSQSSIDQKLANQINDARQTVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 30 - protein, 699 aa, synthetic construct
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPIEVYVNDSVWVPGPIDDRCPAKPEEEGMMINISIGYRYPPICLGRAPGCLMPAVQNWLVEVPTVSPICRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIVSPVSGPEHPELWRLTVASHHIRIWSGNQTLETRDRKPFYTVDLNSSLTVPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDWQKNSTRLWNSQSSIDQKLANQINDLAQTVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 31 - protein, 699 aa, synthetic construct
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPIEVYVNDSVWVPGPIDDRCPAKPEEEGMMINISIGYRYPPICLGRAPGCLMPAVQNWLVEVPTVSPICRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIVSPVSGPEHPELWRLTVASHHIRIWSGNQTLETRDRKPFYTVDLNSSLTVPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDWQKNSTRLWNSQSSIDQKLANQINDLRATVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 32 - protein, 699 aa, synthetic construct
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPIEVYVNDSVWVPGPIDDRCPAKPEEEGMMINISIGYRYPPICLGRAPGCLMPAVQNWLVEVPTVSPICRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIVSPVSGPEHPELWRLTVASHHIRIWSGNQTLETRDRKPFYTVDLNSSLTVPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDWQKNSTRLWNSQSSIDQKLANQINDLRQAVIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 33 - protein, 699 aa, synthetic construct
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPIEVYVNDSVWVPGPIDDRCPAKPEEEGMMINISIGYRYPPICLGRAPGCLMPAVQNWLVEVPTVSPICRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIVSPVSGPEHPELWRLTVASHHIRIWSGNQTLETRDRKPFYTVDLNSSLTVPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDWQKNSTRLWNSQSSIDQKLANQINDLRQTAIWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 34 - protein, 699 aa, synthetic construct
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPIEVYVNDSVWVPGPIDDRCPAKPEEEGMMINISIGYRYPPICLGRAPGCLMPAVQNWLVEVPTVSPICRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIVSPVSGPEHPELWRLTVASHHIRIWSGNQTLETRDRKPFYTVDLNSSLTVPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDWQKNSTRLWNSQSSIDQKLANQINDLRQTVAWMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 35 - protein, 699 aa, synthetic construct
MNPSEMQRKAPPRRRRHRNRAPLTHKMNKMVTSEEQMKLPSTKKAEPPTWAQLKKLTQLATKYLENTKVTQTPESMLLAALMIVSMVVSLPMPAGAAAANYTYWAYVPFPPLIRAVTWMDNPIEVYVNDSVWVPGPIDDRCPAKPEEEGMMINISIGYRYPPICLGRAPGCLMPAVQNWLVEVPTVSPICRFTYHMVSGMSLRPRVNYLQDFSYQRSLKFRPKGKPCPKEIPKESKNTEVLVWEECVANSAVILQNNEFGTIIDWAPRGQFYHNCSGQTQSCPSAQVSPAVDSDLTESLDKHKHKKLQSFYPWEWGEKGISTPRPKIVSPVSGPEHPELWRLTVASHHIRIWSGNQTLETRDRKPFYTVDLNSSLTVPLQSCVKPPYMLVVGNIVIKPDSQTITCENCRLLTCIDSTFNWQHRILLVRAREGVWIPVSMDRPWEASPSVHILTEVLKGVLNRSKRFIFTLIAVIMGLIAVTATAAVAGVALHSSVQSVNFVNDWQKNSTRLWNSQSSIDQKLANQINDLRQTVIAMGDRLMSLEHRFQLQCDWNTSDFCITPQIYNESEHHWDMVRRHLQGREDNLTLDISKLKEQIFEASKAHLNLVPGTEAIAGVADGLANLNPVTWVKTIGSTTIINLILILVCLFCLLLVCRCTQQLRRDSDHRERAMMTMAVLSKRKGGNVGKSKRDQIVTVSV

SEQ ID NO: 36 - RNA, 4624 nt, synthetic construct
  source 1..4624: mol_type = other RNA
gaataaactagtattcttctggtccccacagactcagagagaacccgccaccatgggccagaccaagagcaagatcaagtctaagtacgccagctacctgagcttcatcaagatcctgctgaagcgcggaggcgtgaaggtgtccaccaagaacctgatcaagctgttccagatcatcgagcagttctgcccctggtttcctgagcagggcaccctggatctgaaggactggaagcggatcggcaaagagctgaagcaggccggcagaaagggcaacatcatccctctgaccgtgtggaacgactgggccatcattaaggccgctctggaacccttccagaccgaagaggatagcgtgtccgtgtctgatgcccctggcagctgcatcatcgactgcaacgagaacacccggaagaagtcccagaaagagacagagggcctgcactgcgagtatgtggccgaacctgtgatggctcagagcacccagaacgtggactacaaccagctgcaagaagtgatctaccccgagacactgaagctggaaggcaagggccctgaactcgtgggcccttctgagtctaagcccagaggcacatctcctctgcctgctggacaggtgccagtgacactgcagccccagaaacaagtgaaagagaacaagacccagcctcctgtggcctaccagtattggcctccagccgagctgcagtacagacctcctccagagagccagtacggctaccctggaatgcctcctgctcctcaaggcagagccccttatcctcagcctcctaccagacggctgaaccctacagctcctcctagcagacagggctctgagctgcacgagatcattgacaagagccggaaagagggcgacaccgaggcttggcagtttcccgttacactggaacccatgcctccaggcgaaggcgctcaagaaggcgaacctcctacagtggaagcccggtacaagagcttcagcatcaaaatgctgaaggacatgaaggaaggcgtcaagcagtacggccctaacagcccctacatgcggaccctgctggattctattgcccacggccaccggctgatcccttacgattgggagatcctggccaagtcctctctgagccctagccagttcctgcagttcaagacctggtggatcgacggcgtgcaagaacaagtgcggcggaacagagccgccaatcctcctgtgaacatcgacgccgatcagctgctcggaatcggccagaattggagcaccatctctcagcaggccctgatgcagaacgaggccattgaacaagtccgggccatctgcctgagagcctgggagaagattcaggaccccggcagcacatgccccagctttaataccgttcggcagggcagcaaagagccctatcctgactttgtggcccggctgcaggatgtggcccagaagtctattgccgacgagaaggcccggaaagtgatcgtggaactgatggcctacgagaacgccaatccagagtgccagagcgccatcaagcccctgaagggaaaagtgcctgccggctccgatgtgatcagcgagtacgtgaaggcctgcgacggaattggcggagccatgcacaaagccatgctgatggcacaggccatcactggcgttgtgctcggaggacaagttcggacctttggcggcaagtgctacaactgtggccagatcggacacctgaagaaaaactgccccgtgctgaacaagcagaacatcaccatccaggccaccaccaccggcagagaacctccagatctgtgccctagatgcaagaagggcaagcactgggccagccagtgcagaagcaagttcgacaagaacggccagcctctgagcggcaacgagcaaagaggacagcctcaggctcctcagcagaccggcgcatttccaatccagcctttcgtgccccaaggcttccagggacaacagcctccactgagccaagtgttccagggcattagccagctgcctcagtacaacaactgccctccacctcaggctgccgtgcagcagggatcaggcgctacaaattttagcctgctgaagcaggcaggggatgtggaggaaaaccccggacctatgaaccccagcgagatgcagagaaaggccccacctagacggcggagacacagaaatagagcccctctgacacacaagatgaacaagatggtcaccagcgaggaacagatgaagctgcccagcaccaagaaggccgagcctccaacatgggcccagctgaagaaactgacccagctggccaccaagtacctggaaaacaccaaagtgacccagacacctgagagcatgctgctggccgctctgatgatcgtgtccatggtggtgtccctgcctatgcctgctggtgccgccgctgccaattacacatactgggcctacgtgccctttcctcctctgattcgggccgtgacctggatggacaaccccatcgaagtgtacgtgaacgacagcgtgtgggtgcccggaccaatcgacgatagatgtcccgccaagcctgaggaagagggcatgatgatcaacatcagcatcggctaccggtatcctccaatctgcctgggcagagcccctggctgtcttatgccagctgtgcagaactggctggtggaagtgcctaccgtgtctcccatcagccggttcacctaccacatggtgtccggcatgagcctgcggcctagagtgaactacctgcaggacttcagctaccagcggagcctgaagttcagacccaagggcaagccctgtcctaaagagatccccaaagagtccaagaacaccgaggtgctcgtgtgggaagagtgcgtggccaattctgccgtgatcctgcagaacaacgagttcggcaccatcatcgactgggcccctagaggccagttctaccacaattgcagcggccagacacagagctgccctagcgctcaagtgtcaccagccgtggatagcgatctgaccgagagcctggacaagcacaaacacaagaagctgcagagcttctacccctgggagtggggcgagaagggcatctctacacccagacctaagatcgtgtcacccgtgtccggacctgagcaccctgaactttggagactgacagtggccagccaccacatcagaatttggagcggcaatcagaccctggaaacccgggacagaaagcccttctacaccgtggacctgaacagcagcctgaccgtgcctctccagagctgtgtgaagcctccttacatgctggtcgtgggcaacatcgtgatcaagcccgacagccagaccatcacatgcgagaactgcagactgctgacctgcatcgacagcaccttcaactggcagcaccggatcctgctcgtgcgagctagagaaggcgtgtggatccccgtgtctatggacagaccttgggaagctagccccagcgtgcacatcctgacagaggtgctgaagggcgtgctgaacagaagcaagcggttcatcttcaccctgatcgccgtgatcatgggcctgattgctgtgacagccacagctgctgttgctggcgtggccctgcatagctctgtgcagagcgtgaacttcgtgaacgattggcagaagaacagcacccggctgtggaacagccagagcagcatcgatcagaagctggccaacgccatcaacgacctgcggcagacagtgatctggatgggcgacagactgatgagcctggaacaccggttccagctgcagtgcgactggaataccagcgacttctgcatcacccctcagatctacaacgagagcgagcaccactgggatatggtccgaaggcatctgcagggcagagaggacaacctgacactggacatcagcaagctgaaagagcagatcttcgaggccagcaaggcccacctgaatctggtgcctggaaccgaagctattgccggcgttgcagatggcctggccaatctgaatcctgtgacctgggtcaagaccatcggcagcaccacaatcatcaacctgatcctgatcctcgtgtgcctgttctgcctgctgcttgtgtgcagatgcacccagcagctgagaagagacagcgaccatagagaacgggccatgatgaccatggccgtgctgagcaagagaaaaggcggcaacgtgggcaagagcaagcgggatcagatcgtgaccgtgtccgtgtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 37 - RNA, 4624 nt, synthetic construct
  source 1..4624: mol_type = other RNA
gaataaactagtattcttctggtccccacagactcagagagaacccgccaccatgggccagaccaagagcaagatcaagtctaagtacgccagctacctgagcttcatcaagatcctgctgaagcgcggaggcgtgaaggtgtccaccaagaacctgatcaagctgttccagatcatcgagcagttctgcccctggtttcctgagcagggcaccctggatctgaaggactggaagcggatcggcaaagagctgaagcaggccggcagaaagggcaacatcatccctctgaccgtgtggaacgactgggccatcattaaggccgctctggaacccttccagaccgaagaggatagcgtgtccgtgtctgatgcccctggcagctgcatcatcgactgcaacgagaacacccggaagaagtcccagaaagagacagagggcctgcactgcgagtatgtggccgaacctgtgatggctcagagcacccagaacgtggactacaaccagctgcaagaagtgatctaccccgagacactgaagctggaaggcaagggccctgaactcgtgggcccttctgagtctaagcccagaggcacatctcctctgcctgctggacaggtgccagtgacactgcagccccagaaacaagtgaaagagaacaagacccagcctcctgtggcctaccagtattggcctccagccgagctgcagtacagacctcctccagagagccagtacggctaccctggaatgcctcctgctcctcaaggcagagccccttatcctcagcctcctaccagacggctgaaccctacagctcctcctagcagacagggctctgagctgcacgagatcattgacaagagccggaaagagggcgacaccgaggcttggcagtttcccgttacactggaacccatgcctccaggcgaaggcgctcaagaaggcgaacctcctacagtggaagcccggtacaagagcttcagcatcaaaatgctgaaggacatgaaggaaggcgtcaagcagtacggccctaacagcccctacatgcggaccctgctggattctattgcccacggccaccggctgatcccttacgattgggagatcctggccaagtcctctctgagccctagccagttcctgcagttcaagacctggtggatcgacggcgtgcaagaacaagtgcggcggaacagagccgccaatcctcctgtgaacatcgacgccgatcagctgctcggaatcggccagaattggagcaccatctctcagcaggccctgatgcagaacgaggccattgaacaagtccgggccatctgcctgagagcctgggagaagattcaggaccccggcagcacatgccccagctttaataccgttcggcagggcagcaaagagccctatcctgactttgtggcccggctgcaggatgtggcccagaagtctattgccgacgagaaggcccggaaagtgatcgtggaactgatggcctacgagaacgccaatccagagtgccagagcgccatcaagcccctgaagggaaaagtgcctgccggctccgatgtgatcagcgagtacgtgaaggcctgcgacggaattggcggagccatgcacaaagccatgctgatggcacaggccatcactggcgttgtgctcggaggacaagttcggacctttggcggcaagtgctacaactgtggccagatcggacacctgaagaaaaactgccccgtgctgaacaagcagaacatcaccatccaggccaccaccaccggcagagaacctccagatctgtgccctagatgcaagaagggcaagcactgggccagccagtgcagaagcaagttcgacaagaacggccagcctctgagcggcaacgagcaaagaggacagcctcaggctcctcagcagaccggcgcatttccaatccagcctttcgtgccccaaggcttccagggacaacagcctccactgagccaagtgttccagggcattagccagctgcctcagtacaacaactgccctccacctcaggctgccgtgcagcagggatcaggcgctacaaattttagcctgctgaagcaggcaggggatgtggaggaaaaccccggacctatgaaccccagcgagatgcagagaaaggccccacctagacggcggagacacagaaatagagcccctctgacacacaagatgaacaagatggtcaccagcgaggaacagatgaagctgcccagcaccaagaaggccgagcctccaacatgggcccagctgaagaaactgacccagctggccaccaagtacctggaaaacaccaaagtgacccagacacctgagagcatgctgctggccgctctgatgatcgtgtccatggtggtgtccctgcctatgcctgctggtgccgccgctgccaattacacatactgggcctacgtgccctttcctcctctgattcgggccgtgacctggatggacaaccccatcgaagtgtacgtgaacgacagcgtgtgggtgcccggaccaatcgacgatagatgtcccgccaagcctgaggaagagggcatgatgatcaacatcagcatcggctaccggtatcctccaatctgcctgggcagagcccctggctgtcttatgccagctgtgcagaactggctggtggaagtgcctaccgtgtctcccatcagccggttcacctaccacatggtgtccggcatgagcctgcggcctagagtgaactacctgcaggacttcagctaccagcggagcctgaagttcagacccaagggcaagccctgtcctaaagagatccccaaagagtccaagaacaccgaggtgctcgtgtgggaagagtgcgtggccaattctgccgtgatcctgcagaacaacgagttcggcaccatcatcgactgggcccctagaggccagttctaccacaattgcagcggccagacacagagctgccctagcgctcaagtgtcaccagccgtggatagcgatctgaccgagagcctggacaagcacaaacacaagaagctgcagagcttctacccctgggagtggggcgagaagggcatctctacacccagacctaagatcgtgtcacccgtgtccggacctgagcaccctgaactttggagactgacagtggccagccaccacatcagaatttggagcggcaatcagaccctggaaacccgggacagaaagcccttctacaccgtggacctgaacagcagcctgaccgtgcctctccagagctgtgtgaagcctccttacatgctggtcgtgggcaacatcgtgatcaagcccgacagccagaccatcacatgcgagaactgcagactgctgacctgcatcgacagcaccttcaactggcagcaccggatcctgctcgtgcgagctagagaaggcgtgtggatccccgtgtctatggacagaccttgggaagctagccccagcgtgcacatcctgacagaggtgctgaagggcgtgctgaacagaagcaagcggttcatcttcaccctgatcgccgtgatcatgggcctgattgctgtgacagccacagctgctgttgctggcgtggccctgcatagctctgtgcagagcgtgaacttcgtgaacgattggcagaagaacagcacccggctgtggaacagccagagcagcatcgatcagaagctggccaaccagatcaacgacctgcggcagacagtgatctggatgggcgacagactgatgagcctggaacaccggttccagctgcagtgcgactggaataccagcgacttctgcatcacccctcagatctacaacgagagcgagcaccactgggatatggtccgaaggcatctgcagggcagagaggacaacctgacactggacatcagcaagctgaaagagcagatcttcgaggccagcaaggcccacctgaatctggtgcctggaaccgaagctattgccggcgttgcagatggcctggccaatctgaatcctgtgacctgggtcaagaccatcggcagcaccacaatcatcaacctgatcctgatcctcgtgtgcctgttctgcctgctgcttgtgtgcagatgcacccagcagctgagaagagacagcgaccatagagaacgggccatgatgaccatggccgtgctgagcaagagaaaaggcggcaacgtgggcaagagcaagcgggatcagatcgtgaccgtgtccgtgtgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 38 - protein, 23 aa, synthetic construct
LQNCZGLDLLTAEKGGLCTFLGE

SEQ ID NO: 39 - protein, 23 aa, synthetic construct
AQNRRALDLLTADKGGTCLFLGE

SEQ ID NO: 40 - protein, 23 aa, synthetic construct
LQNRRGLDLLFLSQGGLCAALGE

SEQ ID NO: 41 - protein, 23 aa, synthetic construct
YQNRLALDYLLAAEGGVCGKFNL

SEQ ID NO: 42 - protein, 23 aa, synthetic construct
YQNRLALDYLLAQEGGVCGKFNL

SEQ ID NO: 43 - protein, 23 aa, synthetic construct
MNNRLALDYLLAEQGGVCAVISK

SEQ ID NO: 44 - protein, 23 aa, synthetic construct
MDNRLALDYLLAEQGGVCAVINK

SEQ ID NO: 45 - RNA, 4406 nt, synthetic construct
  source 1..4406: mol_type = other RNA
actcttctggtccccacagactcagagagaagccaccatgggccagaccaagagcaagatcaagtctaagtacgccagctacctgagcttcatcaagatcctgctgaagcgcggaggcgtgaaggtgtccaccaagaacctgatcaagctgttccagatcatcgagcagttctgcccctggtttcctgagcagggcaccctggatctgaaggactggaagcggatcggcaaagagctgaagcaggccggcagaaagggcaacatcatccctctgaccgtgtggaacgactgggccatcattaaggccgctctggaacccttccagaccgaagaggatagcgtgtccgtgtctgatgcccctggcagctgcatcatcgactgcaacgagaacacccggaagaagtcccagaaagagacagagggcctgcactgcgagtatgtggccgaacctgtgatggctcagagcacccagaacgtggactacaaccagctgcaagaagtgatctaccccgagacactgaagctggaaggcaagggccctgaactcgtgggcccttctgagtctaagcccagaggcacatctcctctgcctgctggacaggtgccagtgacactgcagccccagaaacaagtgaaagagaacaagacccagcctcctgtggcctaccagtattggcctccagccgagctgcagtacagacctcctccagagagccagtacggctaccctggaatgcctcctgctcctcaaggcagagccccttatcctcagcctcctaccagacggctgaaccctacagctcctcctagcagacagggctctgagctgcacgagatcattgacaagagccggaaagagggcgacaccgaggcttggcagtttcccgttacactggaacccatgcctccaggcgaaggcgctcaagaaggcgaacctcctacagtggaagcccggtacaagagcttcagcatcaaaatgctgaaggacatgaaggaaggcgtcaagcagtacggccctaacagcccctacatgcggaccctgctggattctattgcccacggccaccggctgatcccttacgattgggagatcctggccaagtcctctctgagccctagccagttcctgcagttcaagacctggtggatcgacggcgtgcaagaacaagtgcggcggaacagagccgccaatcctcctgtgaacatcgacgccgatcagctgctcggaatcggccagaattggagcaccatctctcagcaggccctgatgcagaacgaggccattgaacaagtccgggccatctgcctgagagcctgggagaagattcaggaccccggcagcacatgccccagctttaataccgttcggcagggcagcaaagagccctatcctgactttgtggcccggctgcaggatgtggcccagaagtctattgccgacgagaaggcccggaaagtgatcgtggaactgatggcctacgagaacgccaatccagagtgccagagcgccatcaagcccctgaagggaaaagtgcctgccggctccgatgtgatcagcgagtacgtgaaggcctgcgacggaattggcggagccatgcacaaagccatgctgatggcacaggccatcactggcgttgtgctcggaggacaagttcggacctttggcggcaagtgctacaactgtggccagatcggacacctgaagaaaaactgccccgtgctgaacaagcagaacatcaccatccaggccaccaccaccggcagagaacctccagatctgtgccctagatgcaagaagggcaagcactgggccagccagtgcagaagcaagttcgacaagaacggccagcctctgagcggcaacgagcaaagaggacagcctcaggctcctcagcagaccggcgcatttccaatccagcctttcgtgccccaaggcttccagggacaacagcctccactgagccaagtgttccagggcattagccagctgcctcagtacaacaactgccctccacctcaggctgccgtgcagcagggatcaggcgctacaaattttagcctgctgaagcaggcaggggatgtggaggaaaaccccggacctatgaaccccagcgagatgcagagaaaggccccacctagacggcggagacacagaaatagagcccctctgacacacaagatgaacaagatggtcaccagcgaggaacagatgaagctgcccagcaccaagaaggccgagcctccaacatgggcccagctgaagaaactgacccagctggccaccaagtacctggaaaacaccaaagtgacccagacacctgagagcatgctgctggccgctctgatgatcgtgtccatggtggtgtccctgcctatgcctgctggtgccgccgctgccaattacacatactgggcctacgtgccctttcctcctctgattcgggccgtgacctggatggacaaccccatcgaagtgtacgtgaacgacagcgtgtgggtgcccggaccaatcgacgatagatgtcccgccaagcctgaggaagagggcatgatgatcaacatcagcatcggctaccggtatcctccaatctgcctgggcagagcccctggctgtcttatgccagctgtgcagaactggctggtggaagtgcctaccgtgtctcccatcagccggttcacctaccacatggtgtccggcatgagcctgcggcctagagtgaactacctgcaggacttcagctaccagcggagcctgaagttcagacccaagggcaagccctgtcctaaagagatccccaaagagtccaagaacaccgaggtgctcgtgtgggaagagtgcgtggccaattctgccgtgatcctgcagaacaacgagttcggcaccatcatcgactgggcccctagaggccagttctaccacaattgcagcggccagacacagagctgccctagcgctcaagtgtcaccagccgtggatagcgatctgaccgagagcctggacaagcacaaacacaagaagctgcagagcttctacccctgggagtggggcgagaagggcatctctacacccagacctaagatcgtgtcacccgtgtccggacctgagcaccctgaactttggagactgacagtggccagccaccacatcagaatttggagcggcaatcagaccctggaaacccgggacagaaagcccttctacaccgtggacctgaacagcagcctgaccgtgcctctccagagctgtgtgaagcctccttacatgctggtcgtgggcaacatcgtgatcaagcccgacagccagaccatcacatgcgagaactgcagactgctgacctgcatcgacagcaccttcaactggcagcaccggatcctgctcgtgcgagctagagaaggcgtgtggatccccgtgtctatggacagaccttgggaagctagccccagcgtgcacatcctgacagaggtgctgaagggcgtgctgaacagaagcaagcggttcatcttcaccctgatcgccgtgatcatgggcctgattgctgtgacagccacagctgctgttgctggcgtggccctgcatagctctgtgcagagcgtgaacttcgtgaacgattggcagaagaacagcacccggctgtggaacagccagagcagcatcgatcagaagctggccaacgccatcaacgacctgcggcagacagtgatctggatgggcgacagactgatgagcctggaacaccggttccagctgcagtgcgactggaataccagcgacttctgcatcacccctcagatctacaacgagagcgagcaccactgggatatggtccgaaggcatctgcagggcagagaggacaacctgacactggacatcagcaagctgaaagagcagatcttcgaggccagcaaggcccacctgaatctggtgcctggaaccgaagctattgccggcgttgcagatggcctggccaatctgaatcctgtgacctgggtcaagaccatcggcagcaccacaatcatcaacctgatcctgatcctcgtgtgcctgttctgcctgctgcttgtgtgcagatgcacccagcagctgagaagagacagcgaccatagagaacgggccatgatgaccatggccgtgctgagcaagagaaaaggcggcaacgtgggcaagagcaagcgggatcagatcgtgaccgtgtccgtgtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaa

SEQ ID NO: 46 - protein, 23 aa, synthetic construct
LQNCZGLDLLTAERGGLCTFLGE

SEQ ID NO: 47 - protein, 23 aa, synthetic construct
LQNRRGLDLLFLSRGGLCAFLGE

SEQ ID NO: 48 - protein, 23 aa, synthetic construct
YQNRLALDYLLAARGGVCGFFNL

SEQ ID NO: 49 - protein, 23 aa, synthetic construct
YQNRLALDYLLAQRGGVCGFFNL

SEQ ID NO: 50 - protein, 23 aa, synthetic construct
MNNRLALDYLLAERGGVCAFISK

SEQ ID NO: 51 - protein, 23 aa, synthetic construct
MDNRLALDYLLAERGGVCAFINK